(19) United States
(12) Patent Application Publication
(10) Pub. No.: US 2024/0400620 A1
(43) Pub. Date: Dec. 5, 2024

(54) Compositions Containing Coronavirus Proteins and Epitopes

(71) Applicant: LA JOLLA INSTITUTE FOR IMMUNOLOGY, La Jolla, CA (US)

(72) Inventor: Sujan Shresta, La Jolla, CA (US)

(21) Appl. No.: 18/699,055

(22) PCT Filed: Oct. 14, 2022
(86) PCT No.: PCT/US2022/046682

§ 371 Date: Apr. 5, 2024

Related U.S. Application Data
(60) Provisional application No. 63/255,874, filed on Oct. 14, 2021.
(60) Provisional application No. 63/276,416, filed on Nov. 5, 2021.
(60) Provisional application No. 63/339,345, filed on May 6, 2022.

(51) Int. Cl. C07K 14/005; A61K 39/215; A61P 31/14; A61P 37/04

(52) U.S. Cl. CPC C07K 14/005; A61K 39/215; A61P 31/14; A61P 37/04

(57) ABSTRACT

The present application relates to compositions of matter, processes and uses of compositions of matter relating to Coronavirus proteins, peptides and epitopes, for example, for therapeutic or preventative vaccination against one or more Coronavirus species, subspecies, or strains, and/or for inducing, enhancing, or sustaining an immune response against at least one Coronavirus serotype or species. The Coronavirus may be, for example, SARS-CoV-2, SARS-COV, MERS-COV, OC43, or any coronavirus, including the betacoronaviruses.

### CROSS-REFERENCE TO RELATED APPLICATIONS

This application claims priority to U.S. Provisional Application Ser. No. 63/255,874, filed Oct. 14, 2021, 63/276,416, filed Nov. 5, 2021, and 63/339,345, filed May 6, 2022, all entitled “Compositions Containing Coronavirus Proteins and Epitopes”, the entire contents of which are incorporated herein by reference.

### STATEMENT OF FEDERALLY FUNDED RESEARCH

This invention was made with support under grants U19 AI142790, U01 AI151810, and AI149644, awarded by the National Institutes of Health. The government has certain rights in this invention.

### TECHNICAL FIELD OF THE INVENTION

The present application relates to compositions of matter, processes and use of compositions of matter relating to Coronavirus proteins, peptides and epitopes.

### INCORPORATION-BY-REFERENCE OF MATERIALS FILED ON COMPACT DISC

The present application includes a Sequence Listing which has been submitted in ASCII format via EFS-Web and is hereby incorporated by reference in its entirety. Said ASCII copy, created on ______, 2022, is named ______.xml and is ___,___ bytes in size.

### BACKGROUND OF THE INVENTION

Effective countermeasures against the recent emergence and rapid expansion of the 2019-Novel Coronavirus (SARS-CoV-2, a.k.a. COVID-19, 2019-nCoV, Wuhan-Hu-1, etc.) require the development of data and tools to understand and monitor viral spread and immune responses.

There is an urgent need to address the fundamental gaps in the understanding of Coronaviruses immunology and pathogenesis so as to be able to develop more effective Coronavirus vaccines and/or treatment approaches.

### SUMMARY OF THE INVENTION

This Summary is provided to introduce a selection of concepts in a simplified form that are further described below in the Detailed Description. This Summary is not intended to identify key aspects or essential aspects of the claimed subject matter.

As embodied and described herein, the present disclosure relates to a composition comprising a protein or peptide, or variant, homologue, derivative or subsequence thereof, that comprises, consists or consists essentially of a protein or peptide, or variant, homologue, derivative or subsequence thereof, that comprises, consists or consists essentially of a Coronavirus T cell epitope or a nucleic acid molecule encoding the protein or peptide, or variant, homologue, derivative or subsequence thereof; wherein the composition elicits, stimulates, induces, promotes, increases or enhances a T cell response against two or more different species of Coronavirus. In particular embodiments, the protein or peptide, or variant, homologue, derivative or subsequence thereof elicits, stimulates, induces, promotes, increases or enhances a response against major histocompatibility complex Class II HLA-DRB1*0101. In alternative embodiments, the protein or peptide, or variant, homologue, derivative or subsequence thereof elicits, stimulates, induces, promotes, increases or enhances a response against HLA-B7.

In certain embodiments, the composition elicits, stimulates, induces, promotes, increases or enhances an antibody response against two or more different species of Coronavirus and a T cell response against two or more different species of Coronavirus.

As embodied and broadly described herein, an aspect of the present disclosure relates to proteins or peptides, or variants, homologues, derivatives or subsequences thereof, and comprises, consists or consists essentially of a Coronavirus T cell epitope, and is a Coronavirus spike, nucleoprotein, membrane, receptor-binding domain (RBD), replicase polyprotein 1ab, protein 3a, envelope small membrane protein, non-structural protein 3b, protein 7a, protein 9b, non-structural protein 6, or non-structural protein 8a protein or peptide. In other embodiments, the protein or peptide, or variant, homologue, derivative or subsequence thereof, comprises, consists or consists essentially of a Coronavirus B cell epitope, and is a Coronavirus spike, nucleoprotein, membrane, receptor-binding domain (RBD), replicase polyprotein 1ab, protein 3a, envelope small membrane protein, non-structural protein 3b, protein 7a, protein 9b, non-structural protein 6, or non-structural protein 8a protein or peptide. In alternative embodiments, the protein or peptide, or variant, homologue, derivative or subsequence thereof comprises, consists, or consists essentially of one or more of a Coronavirus spike, nucleoprotein, membrane, or receptor-binding domain (RBD) protein or peptide.

As embodied and broadly described herein, an aspect of the present disclosure relates to compositions that comprise two or more proteins or peptides, or variants, homologues, derivatives or subsequences thereof, from two or more different species of Coronavirus, or nucleic acid molecules encoding two or more proteins or peptides, or variants, homologues, derivatives or subsequences thereof, from two or more different species of Coronavirus, wherein the two or more proteins or peptides, or variants, homologues, derivatives or subsequences thereof, comprise, consist or consist essentially of a Coronavirus T cell epitope. In other embodiments, aspects of the present disclosure relate to compositions that comprise two or more proteins or peptides, or variants, homologues, derivatives or subsequences thereof, from the same species of Coronavirus, or nucleic acid molecules encoding the two or more proteins or peptides, or variants, homologues, derivatives or subsequences thereof, from the same species of Coronavirus, wherein the two or more proteins or peptides, or variants, homologues, derivatives or subsequences thereof, comprise, consist or consist essentially of a Coronavirus T cell epitope.

As embodied and broadly described herein, an aspect of the present disclosure relates to compositions that comprise two or more proteins or peptides, or variants, homologues, derivatives or subsequences thereof, from two or more different species of betacoronavirus, or nucleic acid molecules encoding two or more proteins or peptides, or variants, homologues, derivatives or subsequences thereof, from two or more different species of betacoronavirus, wherein the two or more proteins or peptides, or variants, homologues, derivatives or subsequences thereof, comprise, consist or consist essentially of a betacoronavirus T cell epitope. In other embodiments, an aspect of the present disclosure relates to compositions that comprise two or more proteins or peptides, or variants, homologues, derivatives or subsequences thereof, from the same species of betacoronavirus, or nucleic acid molecules encoding the two or more proteins or peptides, or variants, homologues, derivatives or subsequences thereof, from the same species of betacoronavirus, wherein the two or more proteins or peptides, or variants, homologues, derivatives or subsequences thereof, comprise, consist or consist essentially of a betacoronavirus T cell epitope.

As embodied and broadly described herein, an aspect of the present disclosure relates to compositions that comprise proteins or peptides, or variants, homologues, derivatives or subsequences thereof from two or more coronavirus subspecies, strains, or variants, or nucleic acid molecules encoding the proteins or peptides, or variants, homologues, derivatives or subsequences thereof, from two or more coronavirus subspecies, strains, or variants. In other embodiments, the composition comprises a protein, or variant, homologue, derivatives or subsequence thereof from SARS-CoV-2 virus or OC43 virus, or nucleic acid molecules encoding the protein, or variant, homologue, derivative or subsequence thereof, from SARS-CoV-2 virus or OC43 virus.

As embodied and broadly described herein, an aspect of the present disclosure relates to compositions that comprise at least two of the spike, nucleoprotein, membrane, envelope, ORF1ab, ORF3a, ORF8, or receptor-binding domain (RBD) proteins or peptides, or variants, homologues, derivatives or subsequences thereof, from a Coronavirus species, or nucleic acid molecules encoding the spike, nucleoprotein, membrane, envelope, ORF1ab, ORF3a, ORF8, or receptor-binding domain (RBD) proteins or peptides, or variants, homologues, derivatives or subsequences thereof, from a Coronavirus species. In alternative embodiments, it comprises at least two of the spike, nucleoprotein, membrane, or receptor-binding domain (RBD) proteins or peptides, or variants, homologues, derivatives or subsequences thereof, from a betacoronavirus subspecies or strain, or nucleic acid molecules encoding at least two of the spike, nucleoprotein, membrane, or receptor-binding domain (RBD) proteins or peptides, or variants, homologues, derivatives or subsequences thereof, from a betacoronavirus subspecies or strain.

As embodied and broadly described herein, an aspect of the present disclosure relates to a composition that further comprises at least two of the spike, nucleoprotein, membrane, or receptor-binding domain (RBD) proteins or peptides, or a variants, homologues, derivatives or subsequences thereof, from SARS-CoV-2 virus, or nucleic acid molecules encoding at least two of the spike, nucleoprotein, membrane, or receptor-binding domain (RBD) proteins or peptides, or variants, homologues, derivatives or subsequences thereof, from SARS-CoV-2 virus or OC43 virus.

As embodied and broadly described herein, an aspect of the present disclosure relates to compositions that comprise protein or peptide, or variant, homologue, derivative or subsequence thereof, that comprises, consists or consists essentially of a OC43 sequence. In certain embodiments, the OC43 sequence comprises a OC43 amino acid sequence of the OC43 S, N or M proteins. In certain embodiments, the OC43 protein sequence comprises an amino acid sequence that is at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or at least 96%, or at least 97%, or at least 98%, or at least 99%, identical to any one of an epitope from OC43. In certain embodiments, the OC43 amino acid sequence comprises any one of an epitope from OC43.

As embodied and broadly described herein, an aspect of the present disclosure relates to compositions that comprise proteins or peptides, or variants, homologues, derivatives or subsequences thereof, and comprises, consists or consists essentially of a Coronavirus T cell epitope, DNA vectors and/or DNA vaccine approaches are used to express the aforementioned amino acid sequences. In certain embodiments, the DNA vectors and/or DNA vaccine approaches comprise a nucleic acid sequence that is at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or at least 96%, or at least 97%, or at least 98%, or at least 99%, identical to any one of E₂₆₋₄₀, M₈₆₋₁₀₀, M₁₅₁₋₁₆₅, M₁₆₅₋₁₇₉, M₁₆₁₋₁₇₅, M₁₆₆₋₁₈₀, M₁₇₆₋₁₉₀, M₉₁₋₁₀₅, M₃₆₋₅₀, M₁₄₆₋₁₆₀, M₁₃₆₋₁₅₀, M₁₉₁₋₂₀₅, M₁₁₆₋₁₃₀, M₆₆₋₈₀, M₇₁₋₈₅, N₁₀₇₋₁₂₁, N₃₀₃₋₃₁₇, N₁₂₉₋₁₄₃, N₃₂₈₋₃₄₂, N₃₈₇₋₄₀₁, N₂₁₁₋₂₂₅, N₂₁₆₋₂₃₀, N₈₁₋₉₅, N₃₄₆₋₃₆₀, N₃₅₁₋₃₆₅, N₂₆₁₋₂₇₅, N₂₂₁₋₂₃₅, N₃₁₇₋₃₃₁, N₁₂₆₋₁₄₀, N₃₂₆₋₃₄₀, N₃₀₁₋₃₁₅, N₈₆₋₁₀₀, N₁₀₃₋₁₁₃, N₁₀₃₋₁₁₄, N₁₀₃₋₁₁₅, N₁₀₄₋₁₁₃, N₁₀₄₋₁₁₄, N₁₀₄₋₁₁₅, ORF1ab₅₂₄₆₋₅₂₆₀, ORF1ab₅₀₄₁₋₅₀₅₅, ORF3a₁₀₆₋₁₂₀, ORF3a₁₁₆₋₁₃₀, ORF8₈₆₋₁₀₀, ORF8₄₁₋₅₅, ORF8₉₆₋₁₁₀, ORF8₇₆₋₉₀, ORF8₃₆₋₅₀, ORF8₁₀₁₋₁₁₅, S₃₁₅₋₃₂₉, S₅₁₂₋₅₂₆, S₅₃₀₋₅₄₄, S₅₃₉₋₅₅₃, S₅₄₄₋₅₅₈, S₈₉₅₋₉₀₉, S₉₅₉₋₉₇₃, S₉₉₈₋₁₀₁₂, and S₁₀₄₄₋₁₀₅₈, or as set forth in any one of SEQ ID NOS: 1 to 111, or as set forth in any one of SEQ ID NOS: 1 to 185, multimers, or combinations thereof. In certain embodiments, the nucleic acid sequence comprises any one of E₂₆₋₄₀, M₈₆₋₁₀₀, M₁₅₁₋₁₆₅, M₁₆₅₋₁₇₉, M₁₆₁₋₁₇₅, M₁₆₆₋₁₈₀, M₁₇₆₋₁₉₀, M₉₁₋₁₀₅, M₃₆₋₅₀, M₁₄₆₋₁₆₀, M₁₃₆₋₁₅₀, M₁₉₁₋₂₀₅, M₁₁₆₋₁₃₀, M₆₆₈₀, M₇₁₋₈₅, N₁₀₇₋₁₂₁, N₃₀₃₋₃₁₇, N₁₂₉₋₁₄₃, N₃₂₈₋₃₄₂, N₃₈₇₋₄₀₁, N₂₁₁₋₂₂₅, N₂₁₆₋₂₃₀, N₈₁₋₉₅, N₃₄₆₋₃₆₀, N₃₅₁₋₃₆₅, N₂₆₁₋₂₇₅, N₂₂₁₋₂₃₅, N₃₁₇₋₃₃₁, N₁₂₆₋₁₄₀, N₃₂₆₋₃₄₀, N₃₀₁₋₃₁₅, N₈₆₋₁₀₀, N₁₀₃₋₁₁₃, N₁₀₃₋₁₁₄, N₁₀₃₋₁₁₅, N₁₀₄₋₁₁₃, N₁₀₄₋₁₁₄, N₁₀₄₋₁₁₅, ORF1ab₅₂₄₆₋₅₂₆₀, ORF1ab₅₀₄₁₋₅₀₅₅, ORF3a₁₀₆₋₁₂₀, ORF3a₁₁₆₋₁₃₀, ORF8₈₆₋₁₀₀, ORF8₄₁₋₅₅, ORF8₉₆₋₁₁₀, ORF8₇₆₋₉₀, ORF8₃₆₋₅₀, ORF8₁₀₁₋₁₁₅, S₃₁₅₋₃₂₉, S₅₁₂₋₅₂₆, S₅₃₀₋₅₄₄, S₅₃₉₋₅₅₃, S₅₄₄₋₅₅₈, S₈₉₅₋₉₀₉, S₉₅₉₋₉₇₃, S₉₉₈₋₁₀₁₂, and S₁₀₄₄₋₁₀₅₈, or as set forth in any one of SEQ ID NOS: 1 to 111, or as set forth in any one of SEQ ID NOS: 1 to 185, multimers, or combinations thereof.

As embodied and broadly described herein, an aspect of the present disclosure relates to compositions that comprise a protein, or variant, homologue, derivative or subsequence thereof, that comprises, consists or consists essentially of a consensus or representative amino acid sequence derived from proteins or peptides from two or more different species of Coronavirus or proteins or peptides from two or more different species of Coronavirus, or nucleic acid molecules encoding the consensus or representative amino acid sequence derived from proteins or peptides from two or more different species of Coronavirus or proteins or peptides from two or more different species of Coronavirus. In other embodiments, it comprises a protein, or variant, homologue, derivative or subsequence thereof, that comprises, consists or consists essentially of a consensus or representative sequence derived from proteins or peptides from two or more of SARS-CoV-2, MERS-CoV, SARS-CoV, OC43, or another coronavirus subspecies or strain, or nucleic acid molecules encoding the consensus or representative sequence derived from proteins or peptides from two or more of SARS-CoV-2, MERS-CoV, SARS-CoV, OC43, or another coronavirus subspecies or strain.

As embodied and broadly described herein, an aspect of the present disclosure relates to compositions that comprise a protein, or variant, homologue, derivative or subsequence thereof, that comprises, consists or consists essentially of a consensus or representative sequence derived from the spike, nucleoprotein, membrane, or receptor-binding domain (RBD) proteins or peptides from two or more different species of Coronavirus, or a nucleic acid molecule encoding the protein, or variant, homologue, derivative or subsequence thereof, that comprises, consists or consists essentially of the consensus or representative sequence derived from the spike, nucleoprotein, membrane, or receptor-binding domain (RBD) proteins or peptides from two or more different species of Coronavirus.

As embodied and broadly described herein, an aspect of the present disclosure relates to compositions that comprise a protein, or variant, homologue, derivative or subsequence thereof, that comprises, consists or consists essentially of a consensus or representative sequence derived from proteins or peptides from two or more of SARS-CoV-2, MERS-CoV, SARS-CoV, OC43, or another coronavirus subspecies or strain, or nucleic acid molecules encoding the consensus or representative sequence derived from proteins or peptides from two or more of SARS-CoV-2, MERS-CoV, SARS-CoV, OC43, or another coronavirus subspecies or strain.

As embodied and broadly described herein, an aspect of the present disclosure relates to compositions that comprise a protein, or variant, homologue, derivative or subsequence thereof, that comprises, consists or consists essentially of a consensus or representative sequence derived from the spike, nucleoprotein, membrane or receptor-binding domain proteins or peptides from two or more different species of Coronavirus, or a nucleic acid molecule encoding the protein, or variant, homologue, derivative or subsequence thereof, that comprises, consists or consists essentially of the consensus or representative sequence derived from the spike, nucleoprotein, membrane, envelope, ORF1ab, ORF3a, ORF8, or receptor-binding domain (RBD) proteins or peptides from two or more different species of Coronavirus.

As embodied and broadly described herein, an aspect of the present disclosure relates to compositions that comprise a protein, or variant, homologue, derivative or subsequence thereof, that comprises, consists or consists essentially of a consensus or representative sequence derived from the spike, nucleoprotein, membrane, envelope, ORF1ab, ORF3a, ORF8, or receptor-binding domain (RBD) proteins or peptides from two or more different subspecies, strains, or variants, of betacoronaviruses, or a nucleic acid molecule encoding the protein, or variant, homologue, derivative or subsequence thereof, that comprises, consists or consists essentially of the consensus or representative sequence derived from the spike, nucleoprotein, membrane, envelope, ORF1ab, ORF3a, ORF8, or receptor-binding domain (RBD) proteins or peptides from two or more different subspecies, strains, or variants, of betacoronaviruses.

As embodied and broadly described herein, an aspect of the present disclosure relates to compositions that comprise a protein, or variant, homologue, derivative or subsequence thereof, that comprises, consists or consists essentially of a consensus or representative sequence derived from the spike, nucleoprotein, membrane, envelope, ORF1ab, ORF3a, ORF8, or receptor-binding domain (RBD) proteins or peptides from SARS-CoV-2 and/or OC43 and one or more additional subspecies, strains, or variants, of a coronavirus, or a nucleic acid molecule encoding the protein, or variant, homologue, derivative or subsequence thereof, that comprises, consists or consists essentially of a consensus or representative sequence derived from the spike, nucleoprotein, membrane, envelope, ORF1ab, ORF3a, ORF8, or receptor-binding domain (RBD) proteins or peptides from SARS-CoV-2 and/or OC43 and one or more additional subspecies, strains, or variants, of a coronavirus. In certain embodiments, the proteins or peptides from SARS-CoV-2 and/or OC43 and one or more additional subspecies, strains, or variants of a coronavirus comprise proteins or peptides from two or more species or strains of SARS-CoV-2 and/or OC43.

As embodied and broadly described herein, an aspect of the present disclosure relates to compositions that comprise a protein, or variant, homologue, derivative or subsequence thereof, that comprises, consists or consists essentially of a consensus or representative sequence derived from the spike, nucleoprotein, membrane, envelope, ORF1ab, ORF3a, ORF8, or receptor-binding domain (RBD) proteins or peptides from SARS-CoV and/or SARS-CoV-2 and/or OC43, or a nucleic acid molecule encoding the protein, or variant, homologue, derivative or subsequence thereof, that comprises, consists or consists essentially of a consensus or representative sequence derived from the spike, nucleoprotein, membrane, envelope, ORF1ab, ORF3a, ORF8, or receptor-binding domain (RBD) proteins or peptides from SARS-CoV and/or SARS-CoV-2 and/or OC43.

As embodied and broadly described herein, an aspect of the present disclosure relates to compositions that comprise a protein, or variant, homologue, derivative or subsequence thereof, that comprises, consists or consists essentially of a consensus or representative sequence derived from the spike, nucleoprotein, membrane, envelope, ORF1ab, ORF3a, ORF8, or receptor-binding domain (RBD) proteins or peptides from MERS-CoV and/or SARS-CoV-2 and/or OC43, or a nucleic acid molecule encoding the protein, or variant, homologue, derivative or subsequence thereof, that comprises, consists or consists essentially of a consensus or representative sequence derived from the spike, nucleoprotein, membrane, envelope, ORF1ab, ORF3a, ORF8, or receptor-binding domain (RBD) proteins or peptides from MERS-CoV and/or SARS-CoV-2 and/or OC43.

As embodied and broadly described herein, an aspect of the present disclosure relates to compositions that comprise a CD70 protein or peptide, or variant, homologue, derivative or subsequence thereof, or a nucleic acid molecule encoding a CD70 protein, or variant, homologue, derivative or subsequence thereof. In alternative embodiments, the CD70 protein or peptide is a human CD70 protein or peptide, or variant, homologue, derivative or subsequence thereof, or a nucleic acid molecule encoding a human CD70 protein, or variant, homologue, derivative or subsequence thereof.

As embodied and broadly described herein, an aspect of the present disclosure relates to compositions that further comprise a T cell stimulatory protein or peptide, or variant, homologue, derivative or subsequence thereof, or a nucleic acid molecule encoding a T cell stimulatory protein, or variant, homologue, derivative or subsequence thereof. In certain embodiments, the T cell stimulatory protein or peptide is a human T cell stimulatory protein or peptide, or variant, homologue, derivative or subsequence thereof, or a nucleic acid molecule encoding a human T cell stimulatory protein, or variant, homologue, derivative or subsequence thereof. In certain embodiments, the T cell stimulatory protein comprises OX40L, CD70, 4-1BBL, CD40L, GITRL, ICOS-L/B7RP1, CD80/V71, or CD86/B7-2, or a variant thereof. In certain embodiments, the T cell stimulatory protein comprises an agonist of OX40, CD27, 4-1BB, CD40, GITR, ICOS, or CD28.

As embodied and broadly described herein, an aspect of the present disclosure relates to compositions that comprise the Coronavirus is one or more of a species or subspecies of Embecovirus, Sarbecovirus, Merbecovirus, Nobevovirus, Hibecovirus, SARS-CoV, MERS-CoV, or OC43. In other embodiments, the Coronavirus is one or more of SARS-CoV, SARS-CoV-2, MERS-CoV, SL-CoV-WIV1, HK84, HKU5, HCoV-OC43, HCoV-HKU1, HKU9, or OC43.

As embodied and broadly described herein, an aspect of the present disclosure relates to compositions that further comprise an adjuvant. In some embodiments, the composition comprises one or more vectors configured to direct expression of the protein, or variant, homologue, derivative or subsequence thereof, comprising, consisting or consisting essentially of a Coronavirus T cell epitope. In other embodiments, the composition comprises one or more vectors configured to direct expression of the protein, or variant, homologue, derivative or subsequence thereof that comprises, consists or consists essentially of a Coronavirus B cell epitope. In alternative embodiments, the composition further comprises a vector configured to direct expression of the CD70 protein or the T cell stimulatory protein.

As embodied and broadly described herein, an aspect of the present disclosure relates to a method of eliciting, stimulating, inducing, promoting, increasing, or enhancing an immune response against a Coronavirus, the method comprising administering the composition or a combination of the compositions described herein, either alone or in combination with any compound, agent, drug, treatment or other therapeutic regimen or protocol having a desired therapeutic, beneficial, additive, synergistic or complementary activity or effect in the treatment, prevention, or vaccination against a Coronavirus or the symptoms or side-effects of infection thereof. In certain embodiments, the method elicits, stimulates, induces, promotes, increases, or enhances an immune response against two or more different species of Coronavirus.

As embodied and broadly described herein, the present disclosure is related to a method of vaccinating against, providing a subject with protection against, or treating a subject for a Coronavirus infection, the method comprising administering the composition or a combination of the compositions described herein. In certain embodiments, the method vaccinates against, provides the subject with protection against or treats a subject for infection with two or more different species of a Coronavirus. In alternative embodiments, the method vaccinates against, provides the subject with protection against or treats a subject for infection with two or more different subspecies, strains, or variants of betacoronavirus.

As embodied and broadly described herein, the present disclosure is related to a method of preventing, reducing, or inhibiting the sensitization of a subject to or occurrence in the subject of an antibody dependent enhancement of disease or disease upon a secondary or subsequent Coronavirus infection or following administration of the composition or combination of the compositions described herein, subsequent to a prior Coronavirus infection in the subject or prior to administration to the subject of a vaccine against a Coronavirus.

As embodied and broadly described herein, the present disclosure is related to a method of formulating a vaccine against a Coronavirus that will not elicit, stimulate, induce, promote, increase, enhance or sensitize a subject to an antibody dependent enhancement of disease or infection, the method comprising formulating the vaccine to comprise a composition or a combination of the compositions described herein.

As embodied and broadly described herein, an aspect of the present disclosure relates to a method of inducing, enhancing, or sustaining an immune response against a Coronavirus in a subject may afford one to obtain at least one of the following features: reduce Coronavirus titer, increase or stimulate Coronavirus clearance, reduce or inhibit Coronavirus proliferation, reduce or inhibit increases in Coronavirus titer or Coronavirus proliferation, reduce the amount of a Coronavirus protein or the amount of a Coronavirus nucleic acid, or reduce or inhibit synthesis of a Coronavirus protein or a Coronavirus nucleic acid.

As embodied and broadly described herein, an aspect of the present disclosure relates to a method of inducing, enhancing, or sustaining an immune response against a Coronavirus in a subject includes contacting T cells of the subject with the effective amount of the composition of the present disclosure prior to, substantially contemporaneously with or following exposure to or infection of the subject with the Coronavirus. For example, contacting T cells of the subject with the effective amount of the composition of the present disclosure may occur within 2-72 hours, 2-48 hours, 4-24 hours, 4-18 hours, or 6-12 hours after a rash develops.

As embodied and broadly described herein, an aspect of the present disclosure relates to a nucleic acid vector that expresses the protein or peptide, or variant, homologue, derivative or subsequence thereof, that comprises, consists or consists essentially of a Coronavirus T cell epitope or a nucleic acid molecule encoding the protein or peptide, or variant, homologue, derivative or subsequence thereof.

In one aspect, the Coronavirus is a betacoronavirus. In another aspect, the Coronavirus is SARS-Cov-2. In another aspect, the Coronavirus is OC43. In another aspect, the Coronavirus is SARS-CoV-2 or a betacoronavirus, the herein described method of inducing, enhancing, or sustaining an immune response against a Coronavirus in a subject may treat or mitigate symptoms associated with SARS-CoV-2 and/or betacoronavirus infection such as, but not limited to, fever, rash, headache, cough, tiredness, difficulty breathing, pain behind the eyes, conjunctivitis, muscle or joint pain, nausea, vomiting, loss of appetite, or secondary infection. In another aspect, the composition of the present disclosure may include one or more acceptable carrier selected from the acceptable carriers described herein. For example, an acceptable carrier may be selected from gold particles, sterile water, saline, glucose, dextrose, or buffered solutions. Carriers may include auxiliary agents including, but not limited to, diluents, stabilizers (i.e., sugars and amino acids), preservatives, wetting agents, emulsifying agents, pH buffering agents, viscosity enhancing additives, colors and the like. In another aspect, the composition of the present disclosure may include one or more pharmaceutically acceptable salt selected from the pharmaceutically acceptable salts described herein. For example, a pharmaceutically acceptable salt may be selected from sodium chloride, potassium chloride, sodium sulfate, ammonium sulfate, or sodium citrate. The concentration of the pharmaceutically acceptable salt can be any suitable concentration known in the art, and may be selected from about 10 mM to about 200 mM. In another aspect, the composition may include one or more adjuvant selected from the adjuvants described herein. In different embodiments, an adjuvant can be a naturally occurring adjuvant or a non-naturally occurring adjuvant. For example, an adjuvant may be selected from aluminum hydroxide or mineral oil, and a stimulator of immune responses, such as Bordatella pertussis or Mycobacterium tuberculosis derived proteins. Suitable adjuvants are commercially available as, for example, Freund's Incomplete Adjuvant and Complete Adjuvant (Pifco Laboratories, Detroit, Mich.); Merck Adjuvant 65 (Merck and Company, Inc., Rahway, N.J.); aluminum salts such as aluminum hydroxide gel (alum) or aluminum phosphate; salts of calcium, iron or zinc; an insoluble suspension of acylated tyrosine acylated sugars; cationically or anionically derivatized polysaccharides; polyphosphazenes; biodegradable microspheres; and Quil A. Suitable adjuvants also include, but are not limited to, toll-like receptor (TLR) agonists, particularly toll-like receptor type 4 (TLR-4) agonists (e.g., monophosphoryl lipid A (MPL), synthetic lipid A, lipid A mimetics or analogs), aluminum salts, cytokines, saponins, muramyl dipeptide (MDP) derivatives, CpG oligos, lipopolysaccharide (LPS) of gram-negative bacteria, polyphosphazenes, emulsions, virosomes, cochleates, poly(lactide-co-glycolides) (PLG) microparticles, poloxamer particles, microparticles, liposomes, oil-in-water emulsions, MF59, and squalene. In some embodiments, the adjuvants are not bacterially-derived exotoxins. In one embodiment, adjuvants may include adjuvants which stimulate a Th1 type response such as 3DMPL or QS21. Adjuvants may also include certain synthetic polymers such as poly amino acids and co-polymers of amino acids, saponin, paraffin oil, and muramyl dipeptide. Adjuvants also encompass genetic adjuvants such as immunomodulatory molecules encoded in a co-inoculated DNA, or as CpG oligonucleotides. The co-inoculated DNA can be in the same plasmid construct as the plasmid immunogen or in a separate DNA vector. The reader can refer to Vaccines (Basel). 2015 June; 3(2): 320-343 for further examples of suitable adjuvant.

Additionally or alternatively, the composition of the present disclosure and/or the method of the present disclosure may further include one or more components, such as drugs, immunostimulants (such as α-interferon, β-interferon, γ-interferon, granulocyte macrophage colony stimulator factor (GM-CSF), macrophage colony stimulator factor (M-CSF), and interleukin 2 (IL-2)), antioxidants, surfactants, flavoring agents, volatile oils, buffering agents, dispersants, propellants, and preservatives.

The following exemplification of carriers, modes of administration, dosage forms, etc., are listed as known possibilities from which the carriers, modes of administration, dosage forms, etc., may be selected for use with the present invention. Those of ordinary skill in the art will understand, however, that any given formulation and mode of administration selected should first be tested to determine that it achieves the desired results.

Methods of administration include, but are not limited to, parenteral, e.g., intravenous, intraperitoneal, intramuscular, subcutaneous, mucosal (e.g., oral, intranasal, buccal, vaginal, rectal, intraocular), intrathecal, topical and intradermal routes. Administration can be systemic or local.

The compositions of the present disclosure may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen free water, before use.

For instance, the composition of the present disclosure may be administered in the form of an injectable preparation, such as sterile injectable aqueous or oleaginous suspensions. These suspensions may be formulated according to techniques known in the art using suitable dispersing or wetting agents and suspending agents. The sterile injectable preparations may also be sterile injectable solutions or suspensions in non-toxic parenterally-acceptable diluents or solvents. They may be given parenterally, for example intravenously, intramuscularly or sub-cutaneously by injection, by infusion or per os. Suitable dosages will vary, depending upon factors such as the amount of each of the components in the composition, the desired effect (short or long term), the route of administration, the age and the weight of the subject to be treated. Any other methods well known in the art may be used for administering the composition of the present disclosure.

The composition of the present disclosure may be formulated as a dry powder (i.e., in lyophilized form). Freeze-drying (also named lyophilization) is often used for preservation and storage of biologically active material because of the low temperature exposure during drying. Typically the liquid antigen is freeze dried in the presence of agents to protect the antigen during the lyophilization process and to yield a cake with desirable powder characteristics. Sugars such as sucrose, mannitol, trehalose, or lactose (present at an initial concentration of 10-200 mg/mL) are commonly used for cryoprotection of protein antigens and to yield lyophilized cake with desirable powder characteristics. Lyophilizing the composition theoretically results in a more stable composition.

In certain embodiments, the composition of the present disclosure may be formulated as a liquid (e.g. aqueous formulation), e.g., as syrups or suspensions, or may be presented as a drug product for reconstitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). The pharmaceutical compositions may take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well-known in the art.

Where the composition of the present disclosure is intended for delivery to the respiratory (e.g. nasal) mucosa, typically it is formulated as an aqueous solution for administration as an aerosol or nasal drops, or alternatively, as a dry powder, e.g. for rapid deposition within the nasal passage. Compositions for administration as nasal drops may contain one or more excipients of the type usually included in such compositions, for example preservatives, viscosity adjusting agents, tonicity adjusting agents, buffering agents, and the like. Viscosity agents can be microcrystalline cellulose, chitosan, starches, polysaccharides, and the like. Compositions for administration as dry powder may also contain one or more excipients usually included in such compositions, for example, mucoadhesive agents, bulking agents, and agents to deliver appropriate powder flow and size characteristics. Bulking and powder flow and size agents may include mannitol, sucrose, trehalose, and xylitol.

In one embodiment, the herein described subject can be a mammal, preferably a human.

### BRIEF DESCRIPTION OF FIGURES

All features of exemplary embodiments which are described in this disclosure and are not mutually exclusive can be combined with one another. Elements of one embodiment can be utilized in the other embodiments without further mention. Other aspects and features of the present invention will become apparent to those ordinarily skilled in the art upon review of the following description of specific embodiments in conjunction with any accompanying Figures.

FIGS. 1A to 1E shows the mapping of SARS-CoV-2 S, N, and M protein-derived epitopes in DNA-vaccinated HLA-B*0702 and HLA-DRB1*0101 Ifnar1^{-/-} mice. (FIG. 1A) SARS-CoV-2 genome and DNA vaccine constructs containing mammalian-optimized Kozak sequence, IgE leader sequence, and codon-optimized DNA sequence for SARS-CoV-2 S, N, or M protein. FIG. 1B) Representative immunofluorescence images of 293T cells transfected with S, M, or N DNA vaccines or with empty vector (insets) and immunolabeled for SARS-CoV-2 S, N, or M protein (lighter). Scale bars apply to main panels and insets. (FIG. 1C) Experimental protocol for FIG. 1D and FIG. 1E. Groups of HLA-B*0702 or HLA-DRB1*0101 Ifnar1^{-/-} mice were administered 25 μg S, N, or M DNA vaccines by intramuscular electroporation on days 0 and 14, and spleen and lung tissue were collected on day 21. (FIG. 1D and FIG. 1E) ELISpot quantification of IFNγ-producing cells (spot-forming cells, SFC) from HLA-B*0702 Ifnar^{-/-} mice (FIG. 1D) or HLA-DRB1*0101 Ifnar^{-/-} mice (FIG. 1E). Splenocytes and lung leukocytes were incubated alone (no peptide) or stimulated for 20 h with 62 (FIG. 1D) or 42 (FIG. 1E) SARS-CoV-2 peptides predicted to be immunogenic for CD8⁺ T cells (FIG. 1D) or CD4⁺ T cells (FIG. 1E) (Tables 2 and 3). Data are presented as the mean±SEM of n=4 mice/group pooled from two independent experiments. *P<0.05,**P<0.01 vs no peptide by the nonparametric Kruskal-Wallis test. Horizontal bars indicate peptides that significantly stimulate one or more cell types (CD4⁺ or CD8⁺ splenocytes or lung leukocytes).

FIGS. 2A to 2C shows the mapping of SARS-CoV-2 S, N, and M protein-derived epitopes in SARS-CoV-2-infected HLA-B*0702 and HLA-DRB1*0101 Ifnar1^{-/-} mice. (FIG. 2A) Experimental protocol for B and C. Groups of HLA-B*0702 or HLA-DRB1*0101 Ifnar1^{-/-} mice were infected IN with 104 PFU of SARS-CoV-2 strains B.1.351 or MA10, respectively, and spleens were collected 8 days later. (FIG. 2B and FIG. 2C) ICS analysis of activated CD8⁺ T cells from B.1.351-infected HLA-B*0702 Ifnar1^{-/-} mice (FIG. 2B) or of activated CD4⁺ T cells from MA10-infected HLA-DRB1*0101 Ifnar1^{-/-} mice (FIG. 2C). Splenocytes were stimulated for 6 h with the indicated 6 (FIG. 2B) or 42 (FIG. 2C) SARS-CoV-2 peptides or with no peptide, immunolabeled for cell surface markers, intracellular cytokines, and the degranulation marker CD107a, and analyzed by flow cytometry. Data are presented as the mean±SEM of n=6 (FIG. 2B) or n=9 (FIG. 2C) mice/group pooled from two independent experiments. Circles represent individual mice. *P<0.05, **P<0.01, ***P<0.001 vs no peptide by the nonparametric Kruskal-Wallis test. Horizontal bars indicate peptides that significantly stimulate CD8⁺ or CD4⁺ T cells with at least one secretion phenotype.

FIGS. 3A to 3E show the cross-reactivity of OC43-elicited CD8⁺ and CD4⁺ T cells for SARS-CoV-2 peptides. (FIG. 3A) Experimental protocol for FIG. 3B-FIG. 3E. Groups of HLA-1B*0702 or HLA-DRB1*0101 Ifnar1^{-/-} mice were infected IN with 10⁹ genomic equivalents of OC43, and tissues were collected 8 and 16 days later. (FIG. 3B) ICS analysis of activated CD8⁺ T cells from OC43-infected HLA-B*0702 Ifnar1^{-/-} mice. Splenocytes were stimulated for 6 h with 37 published HLA-B*0702-restricted SARS-CoV-2-derived peptides (Table 4) or with no peptide, immunolabeled for cell surface markers and intracellular cytokines, and analyzed by flow cytometry. Data are presented as the mean±SEM of n=4 mice/group pooled from two independent experiments. Circles represent individual mice. **P<0.01, ***P <0.001 by the nonparametric Kruskal-Wallis test. (FIG. 3C) ELISpot quantification of IFNγ-producing cells (spot-forming cells, SFC) in splenocytes and lung leukocytes from OC43-infected HLA-B*0702 Ifnar1^{-/-} mice. Splenocytes were isolated on day 8 post-infection and stimulated for 20 h with 69 SARS-CoV-2 peptides (Table 2) or with no peptide. Horizontal bars indicate peptides that significantly stimulated either splenocytes or lung leukocytes. Data are presented as the mean±SEM of n=6 mice/group pooled from two independent experiments. *P<0.05 by the nonparametric Kruskal-Wallis test. (FIG. 3D) ICS analysis of activated CD8⁺ T cells from OC43-infected HLA-B*0702 Ifnar1^{-/-} mice at days 1-30 post-infection. Splenocytes (white circles) and lung leukocytes (black circles) were stimulated for 6 h with SARS-CoV-2 N₁₀₄₋₁₁₃ peptide, immunolabeled for cell surface markers, intracellular cytokines, and CD107a, and analyzed by flow cytometry. Data are presented as the mean±SEM of n=3-8 mice/group pooled from two independent experiments. Circles represent individual mice. *P<0.05, **P<0.01, ***P <0.001 by two-way ANOVA with Sidak's multiple comparison test. Bottom, middle, and top levels of asterisks are comparisons vs day 1, day 8, and day 16 data, respectively (where only one level of asterisks appear is vs day 1 data; where only two levels of asterisks appear are vs day 1 and day 16 data). (FIG. 3E) ICS analysis of activated CD4⁺ T cells from OC43-infected HLA-DRB1*0101 Ifnar1^{-/-} mice. Splenocytes isolated at day 8, 16, or 30 post-infection were stimulated for 6 h with the 37 published HLA-DRB1*0101-restricted SARS-CoV-2-derived peptides (Table 5) or with no peptide, immunolabeled for cell surface markers and intracellular cytokines, and analyzed by flow cytometry. Data are presented as the mean±SEM of n=4 mice/group pooled from two independent experiments. Circles represent individual mice. *P<0.05, **P<0.01 by the nonparametric Kruskal-Wallis test.

FIGS. 4A to 4L shows the protective effect of OC43 pre-exposure and SARS-CoV-2 N₁₀₄₋₁₁₃ immunization on SARS-CoV-2 infection and lung disease in HLA-B*0702 Ifnar1^{-/-} mice. (FIG. 4A) Experimental protocol for FIG. 4B to FIG. 4E. Mice were injected with DMSO (mock-immunized) or SARS-CoV-2 N₁₀₄₋₁₁₃ on day 1 (complete Freund's adjuvant, CFA) and day 21 (incomplete Freund's adjuvant, IFA). Both groups of mice were challenged IN with 10⁵ PFU of SARS-CoV-2 B.1.351 2 weeks later and tissues were collected 3 days after SARS-CoV-2 challenge. (FIG. 4B) ICS analysis of activated CD8⁺ T cells. Splenocytes were stimulated for 6 h with SARS-CoV-2 N₁₀₄₋₁₁₃ peptide, immunolabeled for cell surface markers, intracellular cytokines, and CD107a, and analyzed by flow cytometry. n=7 (mock) or n=8 (peptide-immunized) mice/group. (FIG. 4C) Representative H&E-stained sections of lungs. Grey arrows indicate bronchiolar epithelial cells (BEC) with or without cell necrosis, and black arrows indicate epithelial cells within bronchioles. Lung sections were scored from 0 (least severe) to 5 (most severe) for standard histopathological features of SARS-CoV-2-induced lung damage. n=7 (mock) or n=8 (peptide-immunized) mice/group. (FIG. 4D and FIG. 4E) RT-qPCR of genomic SARS-CoV-2 RNA in the lungs. Representative immunofluorescence staining of SARS-CoV-2 N protein (magenta) in lung sections (F). The graph (right) shows the quantification of lung areas positive for N protein staining. Mock, n=7; peptide-immunized, n=8 mice/group. (FIG. 4F) Experimental protocol for FIG. 4G to FIG. 4J. Mice were infected IN with 10⁹ genomic equivalents (GE) of OC43 or PBS (naïve) and challenged IN with 10⁵ PFU of SARS-CoV-2 B.1.351 60-70 days later. Tissues were collected 3 days after B.1.351 challenge. (FIG. 4G) ICS analysis of activated CD8⁺ T cells as described for B. Naïve, n=10; OC43-infected, n=11 mice/group. (FIG. 4H and FIG. 4I) RT-qPCR of genomic SARS-CoV-2 RNA and N protein staining in lung sections as described for D and E. Naïve, n=6 to 11; OC43-infected, n=8 mice/group. (FIG. 4J) Lung histopathology and scoring as described for FIG. 4C. Naïve, n=11; OC43-infected, n=8 mice/group. (K) Experimental protocol for FIG. 4L: Mice were infected IN with 10⁹ GE of OC43 or PBS (naïve) and challenged IN with 10⁵ PFU B.1.351 60-70 days later. Mice were injected intraperitoneally with a CD8⁺ T cell-depleting antibody (α-CD8) or with an isotype control antibody once daily for 3 days immediately before the B.1.351 challenge. Tissues were collected 3 days after challenge. (FIG. 4L) RT-qPCR of genomic SARS-CoV-2 RNA in the lung. N=3 mice/group. Data are presented as the mean±SEM of the indicated number of mice/group pooled from two independent experiments. Circles represent individual mice. *P<0.05, **P<0.01, ***P<0.001 by the Mann-Whitney test.

FIGS. 5A to 5H show the protective effect of OC43 pre-exposure on SARS-CoV-2 infection and lung disease in HLA-DRB1*0101 Ifnar1^{-/-} mice. (FIG. 5A) Experimental protocol for FIG. 5B to FIG. 5D. Mice were infected IN with 10⁹ genomic equivalents (GE) of OC43 or medium (naïve) and challenged with 10⁵ PFU of SARS-CoV-2 B.1.351 16 days later. Lungs were collected 3 days after challenge. (FIG. 5B and FIG. 5C) RT-qPCR of SARS-CoV-2 genomic RNA in the lung and representative immunofluorescence staining of SARS-CoV-2 N protein (lowest arrow) in lung sections. The graph (right) shows the quantification of lung areas positive for N protein staining. (FIG. 5D) Lung histopathology 3 days. Representative H&E-stained sections. Arrows indicate bronchiolar epithelial cells (BEC) with or without cell necrosis, black arrows indicate epithelial cells within bronchioles, and middle arrows indicate perivascular cuffing. Lung sections were scored from 0 (least severe) to 5 (most severe) for standard histopathological features of SARS-CoV-2-induced lung damage. (FIG. 5E) Experimental protocol for FIG. 5F to FIG. 5H. Mice were infected IN with 10⁹ of OC43 or medium (naïve) and challenged IN with 10⁵ PFU of B.1.351 16 days later. Mice were administered intraperitoneal injections of a CD4⁺ T cell-depleting antibody (α-CD4) or isotype control antibody once daily for 3 days immediately before B.1.351 challenge. Lungs were collected 3 days after challenge. (FIG. 5F and FIG. 5G) RT-qPCR and immunofluorescence staining as described for (FIG. 5B and FIG. 5C). (FIG. 5H) Lung histopathology and scoring as described for (FIG. 5D). Data are presented as the mean±SEM of FIG. 5B-FIG. 5D: n=10 (naïve) or n=8 (OC43-infected) mice/group or FIG. 5F-FIG. 5H: n=3-4 mice/group. Data were pooled from 2 or 3 independent experiments. Circles represent individual mice. *P<0.05; ***P<0.001 by the Mann-Whitney test.

FIGS. 6A-6C (related to FIG. 1). Validation of SARS-CoV-2 S, N, and M protein-derived epitopes in vaccinated HLA-B*0702 Ifnar1^{-/-} mice. (FIG. 6A) Experimental protocol. Mice were injected with saline or 25 μg S-, N-, or M-based DNA vaccine via intramuscular electroporation on days 0 and 14, and spleens were collected at 7 days later. (FIG. 6B) Gating strategy used to analyze activated (CD44⁺ CD62L⁻) CD8⁺ T cells producing cytokines (IFNγ, TNF, IL-2) and the degranulation marker CD107a after stimulation of splenocytes with SARS-CoV-2-derived peptides. Cells producing IFNγ⁺/TNF⁺/IL-2⁺ were identified from IFNγ⁺/TNF⁺ cells producing IL-2 using a Boolean algorithm. (FIG. 6C) ICS analysis of activated CD8⁺ T cells. Splenocytes were stimulated for 6 h with the indicated SARS-CoV-2 S, M, or N protein-derived peptides, immunolabeled for cell surface markers, cytokines, and CD107a, and analyzed by flow cytometry. Mean±SEM of n=5-6 mice/group pooled from two independent experiments. Circles represent individual mice.

FIG. 7 (related to FIG. 3). OC43 infection in mice. (FIG. 7A) Experimental protocol for FIG. 7B and FIG. 7C. HLA-B*0702 or HLA-DRB1*0101 Ifnar1^{-/-} mice were infected intranasally (IN) with 10⁹ genomic equivalents (GE) of OC43 and nasal turbinates and lungs were collected on days 1, 3, and 5 post-infection. (FIG. 7B and FIG. 7C) RT-qPCR analysis of genomic OC43 RNA in nasal turbinates and lungs of HLA-B*0702 Ifnar1^{-/-} (FIG. 7B) and HLA-DRB1*01010 Ifnar1^{-/-} (FIG. 7C) mice. N=mice/group. (FIG. 7D) Experimental protocol for FIG. 7E and FIG. 7F. HLA-DRB1*0101 Ifnar1^{-/-} mice were infected IN with 10⁹ GE OC43, and blood samples were collected at the indicated time points pre- and post-infection. (FIG. 7E and FIG. 7F) ELISA analysis of IgG anti-OC43 S protein and anti-SARS-CoV-2 S protein (FIG. 7E), and IgG anti-OC43 N protein and anti-SARS-CoV-2 N protein titers (FIG. 7F) in sera from mice described in D. N=5 mice/group. (FIG. 7G) ICS analysis of activated CD4⁺ T cells from HLA-DRB1*0101 Ifnar1^{-/-} mice infected IN with 10⁹ GE of OC43. Spleens were collected on day 8 (n=3), 16 (n=4), or 30 (n=4) post-infection. Splenocytes were stimulated for 6 h with 37 SARS-CoV-2 peptides (Table 5), immunolabeled for cell surface markers and intracellular cytokines, and analyzed by flow cytometry. Data are presented as the mean±SEM of the indicated number of mice per group pooled from two independent experiments. Circles represent individual mice. *P<0.05, **P<0.01 by the nonparametric Kruskal-Wallis test.

FIG. 8 (related to FIG. 4). Protective effect of SARS-CoV-2 N₁₀₄₋₁₁₃ immunization on SARS-CoV-2 infection and lung disease in HLA-B*0702 Ifnar1^{-/-} mice challenged with SARS-CoV-2 MA10. (FIG. 8A) Experimental protocol for FIG. 8B and FIG. 8C. Mice were injected with DMSO (mock-immunized) or SARS-CoV-2 N₁₀₄₋₁₁₃ on day 1 (complete Freund's adjuvant, CFA) and day 21 (incomplete Freund's adjuvant, IFA). Mice were challenged intranasally (IN) with 105 PFU of SARS-CoV-2 MA10 2 weeks later and tissues were collected 3 days after SARS-CoV-2 challenge. (FIG. 8B) ICS analysis of activated CD8⁺ T cells. Splenocytes were stimulated for 6 h with SARS-CoV-2 N₁₀₄₋₁₁₃ peptide, immunolabeled for cell surface markers, intracellular cytokines, and CD107a, and analyzed by flow cytometry. n=5 mice/group. (FIG. 8C) Representative H&E-stained sections of lung. Grey arrows indicate bronchiolar epithelial cells (BEC) with or without cell necrosis, and black arrows indicate epithelial cells within bronchioles. The sections were scored from 0 (least severe) to 5 (most severe) for standard histopathological features of SARS-CoV-2-induced lung damage. n=5 mice/group.

FIG. 9 (related to FIG. 4). SARS-CoV-2 RNA load and lung pathology of OC43-infected HLA-B*0702 Ifnar1^{-/-} mice challenged with SARS-CoV-2 B.1.351. (FIG. 9A) Experimental protocol. Mice were infected intranasally (IN) with 10⁹ genomic equivalents (GE) of OC43 or medium (naïve) and challenged with SARS-CoV-2 B.1.351 8 or 16 days later. Tissues were collected 3 days after SARS-CoV-2 challenge. (FIG. 9B) RT-qPCR of genomic OC43 RNA in the lungs and nasal turbinates. (FIG. 9C) Quantification of lung histopathology findings. H&E-stained lung sections were scored from 0 (least severe) to 5 (most severe) for standard histopathological features of SARS-CoV-2-induced lung disease. (FIG. 9D) ICS analysis of OC43-elicited activated CD8⁺ T cells. Splenocytes were stimulated for 6 h with N₁₀₄₋₁₁₃ peptide, immunolabeled for cell surface markers, cytokines, and CD107a, and analyzed by flow cytometry. (FIG. 9E) Gating strategy for analysis of CD8⁺ T cells in the blood and spleen of mice treated with a CD8⁺ T cell-depleting antibody (α-CD8) or isotype control antibody. Protocol is as described in FIG. 4M. Data are presented as the mean±SEM of n=4-5 mice/group pooled from two independent experiments. Circles represent individual mice. *P<0.05 by the Mann-Whitney test.

FIG. 10 (related to FIG. 5) shows SARS-CoV-2 challenge of HLA-DRB1*0101 Ifnar1^{-/-} mice pre-exposed to OC43. (FIG. 10A, FIG. 10B) RT-qPCR of SARS-CoV-2 genomic RNA in nasal turbinates. Experimental protocols are shown in FIG. 5A (FIG. 10A) and FIG. 5E (FIG. 10B). Data are presented as the mean±SEM of (FIG. 10A) naïve, n=10; OC43-infected, n=8; and (FIG. 10B) n=4 mice/group. Differences were analyzed by the Mann-Whitney test. (FIG. 10C) Gating strategy for analysis of CD4⁺ T cells in the blood of mice treated with a CD4⁺ T cell-depleting antibody (α-CD4) or isotype control antibody. Protocol is as described in FIG. 5E. Each dot-plot represents a mouse.

### DETAILED DESCRIPTION

A detailed description of one or more embodiments of the invention is provided below along with any accompanying figures that illustrate the principles of the invention. The invention is described in connection with such embodiments, but the invention is not limited to any embodiment. Numerous specific details are set forth in the following description in order to provide a thorough understanding of the invention. These details are provided for the purpose of non-limiting examples and the invention may be practiced according to the claims without some or all of these specific details. For the purpose of clarity, technical material that is known in the technical fields related to the invention has not been described in detail so that the invention is not unnecessarily obscured.

The present application describes experimental results and lines of reasoning which support the development of more effective Coronavirus vaccine and/or treatment approaches. The inventors have discovered particular SARS-CoV-2 epitopes that stimulate an immune response in a subject after vaccination against SARS-CoV-2 and/or infection with SARS-CoV-2 or OC43. Accordingly, pharmaceutical compositions or vaccines comprising, consisting of, or consisting essentially of the epitopes disclosed here in may provide protection against and or treatment for one or more strains of Coronaviruses, including but not limited to SARS-CoV-2 and/or OC43.

In one embodiment, the Coronavirus vaccine and/or treatment approach relates to SARS-COV-2.

In one embodiment, the Coronavirus vaccine and/or treatment approach relates to one or more coronaviruses, including SARS-COV, MERS-COV, SARS-CoV-2, OC43, and/or additional Coronaviruses and/or betacoronaviruses, including any and all mutated sequences, strains, or variants related thereto. As a non-limiting example, it is understood by a person skilled in the art that SARS-CoV-2 has presented as various strains in different parts of the world. The present disclosure is expressly intended to cover such strains.

In one embodiment, the Coronavirus vaccine and/or treatment approach relates to SARS-COV-2, SARS, and additional betacoronaviruses, such as OC43.

The present disclosure relates to compositions that comprise proteins or peptides, or variants, homologues, derivatives or subsequences thereof, and comprises, consists or consists essentially of a Coronavirus T cell epitope, vectors (DNA or RNA) and/or vaccine (peptide, DNA, or RNA) approaches used to express the aforementioned amino acid sequences. In certain embodiments, the peptides, or variants, homologues, derivatives or subsequences thereof, the DNA vectors and/or DNA vaccine approaches comprise, as applicable, an amino acid or a nucleic acid sequence that is at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 90%, or at least 91%, or at least 92%, or at least 93%, or at least 94%, or at least 95%, or at least 96%, or at least 97%, or at least 98%, or at least 99%, identical to any one of E₂₆₋₄₀, M₈₆₋₁₀₀, M₁₅₁₋₁₆₅, M₁₆₅₋₁₇₉, M₁₆₁₋₁₇₅, M₁₆₆₋₁₈₀, M₁₇₆₋₁₉₀, M₉₁₋₁₀₅, M₃₆₋₅₀, M₁₄₆₋₁₆₀, M₁₃₆₋₁₅₀, M₁₉₁₋₂₀₅, M₁₁₆₋₁₃₀, M₆₆₋₈₀, M₇₁₋₈₅, N₁₀₇₋₁₂₁, N₃₀₃₋₃₁₇, N₁₂₉₋₁₄₃, N₃₂₈₋₃₄₂, N₃₈₇₋₄₀₁, N₂₁₁₋₂₂₅, N₂₁₆₋₂₃₀, N₈₁₋₉₅, N₃₄₆₋₃₆₀, N₃₅₁₋₃₆₅, N₂₆₁₋₂₇₅, N₂₂₁₋₂₃₅, N₃₁₇₋₃₃₁, N₁₂₆₋₁₄₀, N₃₂₆₋₃₄₀, N₃₀₁₋₃₁₅, N₈₆₋₁₀₀, N₁₀₃₋₁₁₃, N₁₀₃₋₁₁₄, N₁₀₃₋₁₁₅, N₁₀₄₋₁₁₃, N₁₀₄₋₁₁₄, N₁₀₄₋₁₁₅, ORF1ab₅₂₄₆₋₅₂₆₀, ORF1ab₅₄₁₀₋₅₀₅₅, ORF3a₁₀₆₋₁₂₀, ORF3a1₁₆₀₋₁₃₀, ORF8₈₆₋₁₀₀, ORF8₄₁₋₅₅, ORF8₉₆₋₁₁₀, ORF8₇₆₋₉₀, ORF8₃₆₋₅₀, ORF8₁₀₁₋₁₁₅, S₃₁₅₋₃₂₉, S₅₁₂₋₅₂₆, S₅₃₀₋₅₄₄, S₅₃₉₋₅₅₃, S₅₄₄₋₅₅₈, S₃₉₅₋₉₀₉, S₉₅₉₋₉₇₃, S₉₉₈₋₁₀₁₂, and S₁₀₄₄₋₁₀₅₈, or as set forth in any one of SEQ ID NOS: 1 to 111, or as set forth in any one of SEQ ID NOS: 1 to 185, multimers, or combinations thereof. In certain embodiments, the nucleic acid sequence comprises any one of E₂₆₋₄₀, M₈₆₋₁₀₀, M₁₅₁₋₁₆₅, M₁₆₅₋₁₇₉, M₁₆₁₋₁₇₅, M₁₆₆₋₁₈₀, M₁₇₆₋₁₉₀, M₉₁₋₁₀₅, M₃₆₋₅₀, M₁₄₆₋₁₆₀, M₁₃₆₋₁₅₀, M₁₉₁₋₂₀₅, M₁₁₆₋₁₃₀, M₆₆₋₈₀, M₇₁₋₈₅, N₁₀₇₋₁₂₁, N₃₀₃₋₃₁₇, N₁₂₉₋₁₄₃, N₃₂₈₋₃₄₂, N₃₈₇₋₄₀₁, N₂₁₁₋₂₂₅, N₂₁₆₋₂₃₀, N₈₁₋₉₅, N₃₄₆₋₃₆₀, N₃₅₁₋₃₆₅, N₂₆₁₋₂₇₅, N₂₂₁₋₂₃₅, N₃₁₇₋₃₃₁, N₁₂₆₋₁₄₀, N₃₂₆₋₃₄₀, N₃₀₁₋₃₁₅, N₈₆₋₁₀₀, N₁₀₃₋₁₁₃, N₁₀₃₋₁₁₄, N₁₀₃₋₁₁₅, N₁₀₄₋₁₁₃, N₁₀₄₋₁₁₄, N₁₀₄₋₁₁₅, ORF1ab₅₂₄₆₋₅₂₆₀, ORF1ab₅₀₄₁₋₅₀₅₅, ORF3a₁₀₆₋₁₂₀, ORF3a₁₁₆₋₁₃₀, ORF8₈₆₋₁₀₀, ORF8₄₁₋₅₅, ORF8₉₆₋₁₁₀, ORF8₇₆₋₉₀, ORF8₃₆₋₅₀, ORF8₁₀₁₋₁₁₅, S₃₁₅₋₃₂₉, S₅₁₂₋₅₂₆, S₅₃₀₋₅₄₄, S₅₃₉₋₅₅₃, S₅₄₄₋₅₅₈, S₈₉₅₋₉₀₉, S₉₅₉₋₉₇₃, S₉₉₈₋₁₀₁₂, and S₁₀₄₄₋₁₀₅₈, or as set forth in any one of SEQ ID NOS: 1 to 111, or as set forth in any one of SEQ ID NOS: 1 to 185, multimers, or combinations thereof.

In particular embodiments, the protein or peptide, or variant, homologue, derivative or subsequence thereof elicits, stimulates, induces, promotes, increases or enhances a response against major histocompatibility complex Class II HLA-DRB1*0101.

### Definitions

Unless otherwise defined, all technical and scientific terms used herein have the same meaning as commonly understood by a person of ordinary skill in the art to which the present invention pertains. As used herein, and unless stated otherwise or required otherwise by context, each of the following terms shall have the definition set forth below.

“Administering” an expression vector, nucleic acid molecule, or a delivery vehicle (such as a chitosan nanoparticle) to a cell comprises transducing, transfecting, electroporation, translocating, fusing, phagocytosing, shooting or ballistic methods, etc., i.e., any means by which a protein or nucleic acid can be transported across a cell membrane and preferably into the nucleus of a cell.

As used herein, the term antibody (Ab) dependent enhancement of infection (ADE) refers to a phenomenon in which a subject who has antibodies against coronavirus, due to a previous Coronavirus infection or exposure to Coronavirus or antigen (e.g., vaccination, immunization, receipt of maternal anti-Coronavirus antibodies, etc.), suffers from enhanced or a more severe illness after a secondary or subsequent infection with a Coronavirus, or after a Coronavirus vaccination or immunization. Typically, the more severe symptoms include one or more of hemorrhagic fever/shock syndrome, increased viral load, increased vascular permeability, increased hemorrhagic manifestations, thrombocytopenia, and shock, compared to the acute self-limited illness typically caused by Coronavirus in subjects who have not been vaccinated, immunized or previously infected with Coronavirus. Although not wishing to be bound by any theory, ADE is believed to be a consequence of the presence of serotype cross-reactive antibodies enhancing viral infection of cells resulting in higher viral loads and a more severe illness upon subsequent exposure or infection of the subject to a Coronavirus or antigen. Methods and uses of the invention therefore include methods and uses that do not substantially or detectably cause, elicit or stimulate one or more symptoms characteristic of ADE, or more broadly ADE, in a subject.

In addition to ADE, there may be other adverse symptoms that result from, or be enhanced or more severe, when a subject who has antibodies against Coronavirus (e.g., due to a prior infection, exposure, vaccination, immunization, maternal antibodies etc.) becomes infected with Coronavirus, or receives a Coronavirus vaccination or immunization, as compared to a subject that has not been vaccinated, immunized or previously infected with a Coronavirus. Such adverse symptoms that may result from, or may be enhanced or more severe include, for example, fever, headache, rash, liver damage, diarrhea, nausea, vomiting or abdominal pain. It is intended that the methods and uses of the invention therefore also include methods and uses that do not substantially elicit, enhance or worsen one or more such other adverse symptoms that may be elicted, enhanced or be more severe in a subject who has antibodies against a Coronavirus, as compared to a subject that does not have antibodies against a Coronavirus.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (naturally occurring) form of the cell or express a second copy of a native gene that is otherwise normally or abnormally expressed, under expressed or not expressed at all.

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. The term nucleic acid is used interchangeably with gene, cDNA, mRNA, oligonucleotide, and polynucleotide. The nucleotide sequences are displayed herein in the conventional 5′-3′ orientation.

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins. The polypeptide sequences are displayed herein in the conventional N-terminal to C-terminal orientation.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, carboxyglutamate, and O-phosphoserine. The expression “amino acid analogs” refers to compounds that have the same basic chemical structure as a naturally occurring amino acid, i.e., an alpha carbon that is bound to a hydrogen, a carboxyl group, an amino group, and an R group, e.g., homoserine, norleucine, methionine sulfoxide, methionine, and methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon in an amino acid herein, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid and nucleic acid sequences, individual substitutions, deletions or additions that alter, add or delete a single amino acid or -nucleotide or a small percentage of amino acids or nucleotides in the sequence create a “conservatively modified variant,” where the alteration results in the substitution of an amino acid with a chemically similar amino acid. Conservative substitution tables providing functionally similar amino acids are well known in the art.

For example, the following groups each contain amino acids that are conservative substitutions for one another (see, e.g., Creighton, Proteins (1984) W.H. Freeman, New York, pages 6-20, for a discussion of amino acid properties):
Alanine (A), Glycine (G)
Serine (S), Threonine (T)
Aspartic acid (D), Glutamic acid (E)
Asparagine (N), Glutamine (Q)
Cysteine (C), Methionine (M)
Arginine (E), Lysine (K), Histidine (H)
Isoleucine (I), Leucine (L), Valine (V)
Phenylalanine (F), Tyrosine (Y), Tryptophan (W)

In light of the present disclosure, in particular in view of the experimental data described in the examples of the present text, the person of skill will readily understand which amino acid may be substituted, deleted or added to a given sequence to create a conservatively modified variant comprising an amino acid sequence which is at least at least 95%, or at least 96%, or at least 97%, or at least 98%, or at least 99%, identical to one or more amino acid sequence set forth in Table 1 without undue effort.

“Primers” are isolated nucleic acids that are annealed to a complementary target DNA strand by nucleic acid hybridization to form a hybrid between the primer and the target DNA strand, then extended along the target DNA strand by a polymerase, e.g., a DNA polymerase. Primer pairs of the present invention refer to their use for amplification of a target nucleic acid sequence, e.g., by the polymerase chain reaction (PCR) or other conventional nucleic-acid amplification methods, such as qPCR.

The phrases “coding sequence,” “structural sequence,” and “structural nucleic acid sequence” refer to a physical structure comprising an orderly arrangement of nucleic acids. The nucleic acids are arranged in a series of nucleic acid triplets that each form a codon. Each codon encodes for a specific amino acid. Thus, the coding sequence, structural sequence, and structural nucleic acid sequence encode a series of amino acids forming a protein, polypeptide, or peptide sequence. The coding sequence, structural sequence, and structural nucleic acid sequence may be contained within a larger nucleic acid molecule, vector, or the like. In addition, the orderly arrangement of nucleic acids in these sequences may be depicted in the form of a sequence listing, figure, table, electronic medium, or the like.

The phrases “DNA sequence,” “nucleic acid sequence,” and “nucleic acid molecule” refer to a physical structure comprising an orderly arrangement of nucleic acids. The DNA sequence or nucleic acid sequence may be contained within a larger nucleic acid molecule, vector, or the like. In addition, the orderly arrangement of nucleic acids in these sequences may be depicted in the form of a sequence listing, figure, table, electronic medium, or the like.

The term “expression” refers to the transcription of a gene to produce the corresponding mRNA and translation of this mRNA to produce the corresponding gene product (i.e., a peptide, polypeptide, or protein).

The term “isolated” refers to material, such as a nucleic acid or a protein, which is: (1) substantially or essentially free from components which normally accompany or interact with the material as found in its naturally occurring environment or (2) if the material is in its natural environment, the material has been altered by deliberate human intervention to a composition and/or placed at a locus in the cell other than the locus native to the material.

The term “treating” or “treatment” refers to a process by which an infection or a disease or the symptoms of an infection or a disease associated with a Coronavirus strain are prevented, alleviated or completely eliminated. As used herein, the term “prevented” or “preventing” refers to a process by which an infection or a disease or symptoms of an infection or a disease associated with a Coronavirus are obstructed or delayed.

In accordance with the invention, treatment methods are provided that include therapeutic (following infection) and prophylactic (prior to Coronavirus exposure, infection or pathology) methods. For example, therapeutic and prophylactic methods of treating a subject for a Coronavirus infection include treatment of a subject having or at risk of having a Coronavirus infection or pathology, treating a subject with a Coronavirus infection, and methods of protecting a subject from a Coronavirus infection (e.g., provide the subject with protection against Coronavirus infection), to decrease or reduce the probability of a Coronavirus infection in a subject, to decrease or reduce susceptibility of a subject to a Coronavirus infection, or to inhibit or prevent a Coronavirus infection in a subject, and to decrease, reduce, inhibit or suppress transmission of the Coronavirus from a host (e.g., a mosquito) to a subject.

Such methods include administering Coronavirus protein, peptide, or a variant, modification, homologue, derivative or subsequence thereof to therapeutically or prophylactically treat (vaccinate or immunize) a subject having or at risk of having a Coronavirus infection or pathology. Accordingly, methods can treat the Coronavirus infection or pathology, or provide the subject with protection from infection (e.g., prophylactic protection).

In one embodiment, a method includes administering to a subject an amount of Coronavirus protein, peptide, or a variant, modification, homologue, derivative or subsequence thereof sufficient to treat the subject for the Coronavirus infection or pathology. In another embodiment, a method includes administering to a subject an amount of a Coronavirus B cell epitope and/or T cell epitope sufficient to provide the subject with protection against the Coronavirus infection or pathology, or one or more physiological conditions, disorders, illness, diseases or symptoms caused by or associated with the virus infection or pathology. In a further embodiment, a method includes administering a subject an amount of a Coronavirus B cell epitope and/or T cell epitope sufficient to treat the subject for the Coronavirus infection.

In one embodiment, a method comprises administering an amount of Coronavirus proteins, peptides, or a variant, modification, homologue, derivative or subsequence thereof to include B cell epitopes and/or T cell epitopes. In one embodiment, a method includes administering an amount of Coronavirus protein, peptide, or a variant, modification, homologue, derivative or subsequence thereof (e.g., a B cell and/or T cell epitope) to a subject in need thereof, sufficient to provide the subject with protection against Coronavirus infection or pathology. In another embodiment, a method includes administering an amount of a Coronavirus protein, peptide, or a variant, modification, homologue, derivative or subsequence thereof (e.g., a B cell epitope and/or T cell epitope) to a subject in need thereof sufficient to treat, vaccinate or immunize the subject against the Coronavirus infection or pathology.

In accordance with the invention, methods of inducing, increasing, promoting or stimulating anti-Coronavirus activity of T cells in a subject are provided. In one embodiment, a method includes administering to a subject an amount of a Coronavirus T cell epitope sufficient to induce, increase, promote or stimulate anti-Coronavirus activity of T cells in the subject.

In accordance with the invention, methods of inducing, increasing, promoting or stimulating anti-Coronavirus activity of CD8⁺ T cells or CD4⁺ T cells in a subject are provided. In one embodiment, a method includes administering to a subject an amount of a Coronavirus T cell epitope sufficient to induce, increase, promote or stimulate anti-Coronavirus activity of CD8⁺ T cells or CD4⁺ T cells in the subject.

In accordance with the invention, methods of inducing, increasing, promoting or stimulating anti-Coronavirus activity of B cells in a subject are provided. In one embodiment, a method includes administering to a subject an amount of a Coronavirus B cell epitope sufficient to induce, increase, promote or stimulate anti-Coronavirus activity of B cells in the subject.

In methods of the invention, any appropriate Coronavirus protein, peptide, or a variant, modification, homologue, derivative or subsequence thereof can be administered. Non-limiting examples include Coronavirus peptide, subsequence, portion or modification thereof of a SARS-COV-2 or SARS-CoV, or MERS-CoV, or OC43. Additional, non-limiting examples include a Corona or SARS-CoV-2 virus protein (e.g., spike (S), membrane (M) nucleoprotein (N)), or receptor-binding domain (RBD) T cell epitope, such as a subsequence, portion or modification of a sequence in such proteins.

In particular methods embodiments, one or more disorders, diseases, physiological conditions, pathologies and symptoms associated with or caused by a Coronavirus infection or pathology will respond to treatment. In particular methods embodiments, treatment methods reduce, decrease, suppress, limit, control or inhibit Coronavirus numbers or titer; reduce, decrease, suppress, limit, control or inhibit pathogen proliferation or replication; reduce, decrease, suppress, limit, control or inhibit the amount of a pathogen protein; or reduce, decrease, suppress, limit, control or inhibit the amount of a Coronavirus nucleic acid. In additional particular methods embodiments, treatment methods include an amount of a Coronavirus peptide, subsequence or portion thereof sufficient to increase, induce, enhance, augment, promote or stimulate an immune response against a Coronavirus; increase, induce, enhance, augment, promote or stimulate Coronavirus clearance or removal; or decrease, reduce, inhibit, suppress, prevent, control, or limit transmission of Coronavirus to a subject (e.g., transmission from a host to a subject). In further particular methods embodiments, treatment methods include an amount of Coronavirus peptide, subsequence or portion thereof sufficient to protect a subject from a Coronavirus infection or pathology, or reduce, decrease, limit, control or inhibit susceptibility to Coronavirus infection or pathology.

Methods of the invention include treatment methods, which result in any therapeutic or beneficial effect. In various methods embodiments, Coronavirus infection, proliferation or pathogenesis is reduced, decreased, inhibited, limited, delayed or prevented, or a method decreases, reduces, inhibits, suppresses, prevents, controls or limits one or more adverse (e.g., physical) symptoms, disorders, illnesses, diseases or complications caused by or associated with Coronavirus infection, proliferation or replication, or pathology (e.g., fever, rash, headache, cough, tiredness, difficulty breathing, pain behind the eyes, conjunctivitis, muscle or joint pain, nausea, vomiting, loss of appetite, or secondary infection). In additional various particular embodiments, treatment methods include reducing, decreasing, inhibiting, delaying or preventing onset, progression, frequency, duration, severity, probability or susceptibility of one or more adverse symptoms, disorders, illnesses, diseases or complications caused by or associated with Coronavirus infection, proliferation or replication, or pathology (e.g., fever, rash, headache, cough, tiredness, difficulty breathing, pain behind the eyes, conjunctivitis, muscle or joint pain, nausea, vomiting, loss of appetite, or secondary infection). In further various particular embodiments, treatment methods include improving, accelerating, facilitating, enhancing, augmenting, or hastening recovery of a subject from a Coronavirus infection or pathogenesis, or one or more adverse symptoms, disorders, illnesses, diseases or complications caused by or associated with Coronavirus infection, proliferation or replication, or pathology (e.g., fever, rash, headache, cough, tiredness, difficulty breathing, pain behind the eyes, conjunctivitis, muscle or joint pain, nausea, vomiting, loss of appetite, or secondary infection). In yet additional various embodiments, treatment methods include stabilizing infection, proliferation, replication, pathogenesis, or an adverse symptom, disorder, illness, disease or complication caused by or associated with Coronavirus infection, proliferation or replication, or pathology, or decreasing, reducing, inhibiting, suppressing, limiting or controlling transmission of Coronavirus from a to an uninfected subject.

A therapeutic or beneficial effect of treatment is therefore any objective or subjective measurable or detectable improvement or benefit provided to a particular subject. A therapeutic or beneficial effect can but need not be complete ablation of all or any particular adverse symptom, disorder, illness, disease or complication caused by or associated with Coronavirus infection, proliferation or replication, or pathology (e.g., fever, rash, headache, cough, tiredness, difficulty breathing, pain behind the eyes, conjunctivitis, muscle or joint pain, nausea, vomiting, loss of appetite, or secondary infection). Thus, a satisfactory clinical endpoint is achieved when there is an incremental improvement or a partial reduction in an adverse symptom, disorder, illness, disease or complication caused by or associated with Coronavirus infection, proliferation or replication, or pathology, or an inhibition, decrease, reduction, suppression, prevention, limit or control of worsening or progression of one or more adverse symptoms, disorders, illnesses, diseases or complications caused by or associated with Coronavirus infection, Coronavirus numbers, titers, proliferation or replication, Coronavirus protein or nucleic acid, or Coronavirus pathology, over a short or long duration (hours, days, weeks, months, etc.).

A therapeutic or beneficial effect also includes reducing or eliminating the need, dosage frequency or amount of a second active such as another drug or other agent (e.g., anti-viral) used for treating a subject having or at risk of having a Coronavirus infection or pathology. For example, reducing an amount of an adjunct therapy, for example, a reduction or decrease of a treatment for a Coronavirus infection or pathology, or a vaccination or immunization protocol is considered a beneficial effect. In addition, reducing or decreasing an amount of a Coronavirus antigen used for vaccination or immunization of a subject to provide protection to the subject is considered a beneficial effect.

Adverse symptoms and complications associated with Coronavirus infection and pathology include, for example, e.g., fever, rash, headache, cough, tiredness, difficulty breathing, pain behind the eyes, conjunctivitis, muscle or joint pain, nausea, vomiting, loss of appetite, or secondary infection. Other symptoms of Coronavirus infection or pathogenesis are known to one of skill in the art and treatment thereof in accordance with the invention is provided. Thus, the aforementioned symptoms and complications are treatable in accordance with the invention.

Methods and compositions of the invention also include increasing, stimulating, promoting, enhancing, inducing or augmenting an anti-Coronavirus and/or anti-SARS-COV-2 B cell, CD4⁺ and/or CD8⁺ T cell responses in a subject, such as a subject with or at risk of a Coronavirus or SARS-CoV-2 virus infection or pathology. In one embodiment, a method includes administering to a subject an amount of Coronavirus protein, peptide, or a variant, modification, homologue, derivative or subsequence thereof sufficient to increase, stimulate, promote, enhance, augment or induce anti-Coronavirus and/or anti-SARS-COV-2 B cell, CD4⁺ and/or CD8⁺ T cell response in the subject. In another embodiment, a method includes administering to a subject an amount of Coronavirus protein, peptide, or a variant, modification, homologue, derivative or subsequence thereof and administering a Coronavirus antigen, live or attenuated Coronavirus, or a nucleic acid encoding all or a portion (e.g., a B cell or T cell epitope) of any protein or proteinaceous Coronavirus antigen sufficient to increase, stimulate, promote, enhance, augment or induce anti-Coronavirus B cell, CD4⁺ T cell and/or CD8⁺ T cell response in the subject.

Methods of the invention additionally include, among other things, increasing production of a Th1 cytokine (e.g., IFN-gamma, TNF-alpha, IL-1alpha, IL-2, IL-6, IL-8, etc.) or other signaling molecule (e.g., CD40L) in vitro or in vivo. In one embodiment, a method includes administering to a subject in need thereof an amount of Coronavirus protein, peptide, or a variant, modification, homologue, derivative or subsequence thereof sufficient to increase production of a Th1 cytokine in the subject (e.g., IFN-gamma, TNF-alpha, IL-1alpha, IL-2, IL-6, IL-8, etc.) or other signaling molecule (e.g., CD40L).

Methods of the invention additionally include, among other things, decreasing production of a Th1 cytokine (e.g., IFN-gamma, TNF-alpha, IL-1alpha, IL-2, IL-6, IL-8, etc.) or other signaling molecule (e.g., CD40L) in vitro or in vivo where Coronavirus infection has become severe and a subject is suffering from an adverse immune response. In one embodiment, a method includes administering to a subject in need thereof a composition sufficient to decrease production of a Th1 cytokine in the subject (e.g., IFN-gamma, TNF-alpha, IL-1alpha, IL-2, IL-6, IL-8, etc.) or other signaling molecule (e.g., CD40L).

Methods, uses and compositions of the invention include administration of Coronavirus, protein, peptide, or a variant, modification, homologue, derivative or subsequence thereof to a subject prior to contact, exposure or infection by a Coronavirus (e.g. Coronavirus or SARS-CoV-2 virus), administration prior to, substantially contemporaneously with or after a subject has been contacted by, exposed to or infected with a Coronavirus (e.g. Coronavirus or SARS-CoV-2 virus), and administration prior to, substantially contemporaneously with or after Coronavirus (e.g. Coronavirus or SARS-CoV-2 virus) pathology or development of one or more adverse symptoms, disorders, illness or diseases caused by or associated with a Coronavirus infection, or pathology. A subject infected with a Coronavirus may have an infection over a period of 1-5, 5-10, 10-20, 20-30, 30-50, 50-100 hours, days, months, or years.

Invention compositions (e.g., Coronavirus protein peptide, or a variant, modification, homologue, derivative or subsequence thereof, including B cell epitopes and T cell epitopes) and uses and methods can be combined with any compound, agent, drug, treatment or other therapeutic regimen or protocol having a desired therapeutic, beneficial, additive, synergistic or complementary activity or effect. Exemplary combination compositions and treatments include multiple T cell epitopes as set for the herein, second actives, such as anti-Coronavirus compounds, agents and drugs, as well as agents that assist, promote, stimulate or enhance efficacy. Such anti-Coronavirus drugs, agents, treatments and therapies can be administered or performed prior to, substantially contemporaneously with or following any other method of the invention, for example, a therapeutic method of treating a subject for a Coronavirus infection or pathology, or a method of prophylactic treatment of a subject for a Coronavirus infection.

Coronavirus proteins, peptides, or variants, modifications, homologues, derivatives or subsequences thereof can be administered as a combination composition, or administered separately, such as concurrently or in series or sequentially (prior to or following) administering a second active, to a subject. The invention therefore provides combinations in which a method or use of the invention is used in a combination with any compound, agent, drug, therapeutic regimen, treatment protocol, process, remedy or composition, such as an anti-viral (e.g., Coronavirus) or immune stimulating, enhancing or augmenting protocol, or pathogen vaccination or immunization (e.g., prophylaxis) set forth herein or known in the art. The compound, agent, drug, therapeutic regimen, treatment protocol, process, remedy or composition can be administered or performed prior to, substantially contemporaneously with or following administration of one or more Coronavirus proteins, peptides, or variants, modifications, homologues, derivatives or subsequences thereof, or a nucleic acid encoding all or a portion (e.g., a B cell or T cell epitope) of a Coronavirus protein, peptide, or a variant, modification, homologue, derivative or subsequence thereof, to a subject. Specific non-limiting examples of combination embodiments therefore include the foregoing or other compound, agent, drug, therapeutic regimen, treatment protocol, process, remedy or composition.

An exemplary combination is a Coronavirus protein, peptide, variant, modification, homologue, derivative or subsequence thereof (e.g., a B cell, CD4⁺ T cell, or CD8⁺ T cell epitope) and a different Coronavirus protein, peptide, variant, modification, homologue, derivative or subsequence thereof (e.g., a different B or T cell epitope) such as a B cell epitope, T cell epitope, antigen (e.g., Coronavirus extract), or live or attenuated Coronavirus (e.g., inactivated Coronavirus). Such Coronavirus antigens and epitopes set forth herein or known to one skilled in the art include a Coronavirus antigen that increases, stimulates, enhances, promotes, augments or induces a proinflammatory or adaptive immune response, numbers or activation of an immune cell (e.g., T cell, natural killer T (NKT) cell, dendritic cell (DC), B cell, macrophage, neutrophil, eosinophil, mast cell, CD4⁺ or a CD8⁺ cell, B220⁺ cell, CD14⁺, CD11b⁺ or CD11c⁺ cells), an anti-Coronavirus B cell, CD4⁺ T cell or CD8⁺ T cell response, production of a Th1 cytokine, a T cell mediated immune response, a B cell mediated immune response etc.

Combination methods and use embodiments include, for example, second actives such as anti-pathogen drugs, such as protease inhibitors, reverse transcriptase inhibitors, virus fusion inhibitors and virus entry inhibitors, antibodies to pathogen proteins, live or attenuated pathogen, or a nucleic acid encoding all or a portion (e.g., an epitope) of any protein or proteinaceous pathogen antigen, immune stimulating agents, etc., and include contact with, administration in vitro or in vivo, with another compound, agent, treatment or therapeutic regimen appropriate for pathogen infection, vaccination or immunization

In certain instances, as will be apparent to a person of skill in the art, references to a Coronavirus protein, peptide, or a variant, modification, homologue, derivative or subsequence thereof as used herein also encompasses a nucleic acid molecule encoding the Coronavirus protein, peptide, or the variant, modification, homologue, derivative or subsequence thereof. For example, descriptions methods and composition of the present invention comprising administration of a Coronavirus protein, peptide, or a variant, modification, homologue, derivative or subsequence thereof encompasses administration of a nucleic acid molecule encoding the Coronavirus protein, peptide, or the variant, modification, homologue, derivative or subsequence thereof.

Methods of the invention also include, among other things, methods that result in a reduced need or use of another compound, agent, drug, therapeutic regimen, treatment protocol, process, or remedy. For example, for a Coronavirus infection or pathology, vaccination or immunization, a method of the invention has a therapeutic benefit if in a given subject a less frequent or reduced dose or elimination of an anti-Coronavirus treatment results. Thus, in accordance with the invention, methods of reducing need or use of a treatment or therapy for a Coronavirus infection or pathology, or vaccination or immunization, are provided.

In invention methods in which there is a desired outcome, such as a therapeutic or prophylactic method that provides a benefit from treatment, vaccination or immunization Coronavirus protein, peptide, or a variant, modification, homologue, derivative or subsequence thereof can be administered in a sufficient or effective amount. As used herein, a “sufficient amount” or “effective amount” or an “amount sufficient” or an “amount effective” refers to an amount that provides, in single (e.g., primary) or multiple (e.g., booster) doses, alone or in combination with one or more other compounds, treatments, therapeutic regimens or agents (e.g., a drug), a long term or a short term detectable or measurable improvement in a given subject or any objective or subjective benefit to a given subject of any degree or for any time period or duration (e.g., for minutes, hours, days, months, years, or cured).

An amount sufficient or an amount effective can but need not be provided in a single administration and can but need not be achieved by administration of a Coronavirus protein, peptide, or a variant, modification, homologue, derivative or subsequence thereof alone or in a combination composition or method that includes a second active. In addition, an amount sufficient or an amount effective need not be sufficient or effective if given in single or multiple doses without a second or additional administration or dosage, since additional doses, amounts or duration above and beyond such doses, or additional antigens, compounds, drugs, agents, treatment or therapeutic regimens may be included in order to provide a given subject with a detectable or measurable improvement or benefit to the subject. For example, to increase, enhance, improve or optimize immunization and/or vaccination, after an initial or primary administration of one or more Coronavirus proteins peptides, or variants, modifications, homologues, derivatives or subsequences thereof to a subject, the subject can be administered one or more additional “boosters” of one or more Coronavirus peptides, subsequences, portions or modifications thereof. Such subsequent “booster” administrations can be of the same or a different formulation, dose or concentration, route, etc.

An amount sufficient or an amount effective need not be therapeutically or prophylactically effective in each and every subject treated, nor a majority of subjects treated in a given group or population. An amount sufficient or an amount effective means sufficiency or effectiveness in a particular subject, not a group of subjects or the general population. As is typical for such methods, different subjects will exhibit varied responses to treatment.

The expression “an acceptable carrier” may refer to a vehicle for containing a compound that can be administered to a subject without significant adverse effects.

As used herein, the term “adjuvant” means a substance added to the composition of the invention to increase the composition's immunogenicity. The mechanism of how an adjuvant operates is not entirely known. Some adjuvants are believed to enhance the immune response (humoral and/or cellular response) by slowly releasing the antigen, while other adjuvants are strongly immunogenic in their own right and are believed to function synergistically.

The expression “ELISPOT” refers to the known Enzyme-Linked ImmunoSpot assay which typically allows visualization of the secretory product(s) of individual activated or responding cells. Each spot that develops in the assay represents a single reactive cell. Thus, the ELISPOT assay provides both qualitative (regarding the specific cytokine or other secreted immune molecule) and quantitative (the frequency of responding cells within the test population) information. Generally speaking, in an ELISPOT assay, the membrane surfaces in a 96-well PVDF-membrane microtiter plate are coated with capture antibody that binds a specific epitope of the cytokine being assayed. During the cell incubation and stimulation step, a biological sample (typically containing PBMCs) is seeded into the wells of the plate along with the antigen (which can be a peptide as described in the present disclosure), and forms a monolayer on the membrane surface of the well. As the antigen-specific cells are activated, they release the cytokine, which is captured directly on the membrane surface by the immobilized antibody. The cytokine is thus “captured” in the area directly surrounding the secreting cell, before it has a chance to diffuse into the culture media, or to be degraded by proteases and bound by receptors on bystander cells. Subsequent detection steps visualize the immobilized cytokine as an ImmunoSpot; essentially the secretory footprint of the activated cell.

The terms “determining,” “measuring,” “evaluating,” “assessing,” and “assaying,” as used herein, generally refer to any form of measurement, and include determining if an element is present or not in a biological sample. These terms include both quantitative and/or qualitative determinations, which both require sample processing and transformation steps of the biological sample. Assessing may be relative or absolute. The phrase “assessing the presence of” can include determining the amount of something present, as well as determining whether it is present or absent.

The expression “biological sample” includes in the present disclosure any biological sample that is suspected of comprising a T cell, such as for example but without being limited thereto, blood and fractions thereof, urine, excreta, semen, seminal fluid, seminal plasma, prostatic fluid, pre-ejaculatory fluid (Cowper's fluid), pleural effusion, tears, saliva, sputum, sweat, biopsy, ascites, amniotic fluid, lymph, vaginal secretions, endometrial secretions, gastrointestinal secretions, bronchial secretions, breast secretions, and the like. In one non-limiting embodiment, a herein described biological sample can be obtained by any known technique, for example by drawing, by non-invasive techniques, or from sample collections or banks, etc.

The expression “treatment” includes inducing, enhancing, or sustaining an immune response against a Coronavirus infection or symptoms associated thereto. For example, the treatment may induce, increase, promote or stimulate anti-Coronavirus activity of immune system cells in a subject following the treatment. For example, the immune system cells may include T cells, including CD4⁺ T cells, CD8⁺ T cells, and/or B cells.

The expression “therapeutically effective amount” may include the amount necessary to allow the component or composition to which it refers to perform its immunological role without causing overly negative effects in the host to which the component or composition is administered. The exact amount of the components to be used or the composition to be administered will vary according to factors such as the type of condition being treated, the type and age of the subject to be treated, the mode of administration, as well as the other ingredients in the composition.

The term “OC43” refers to coronavirus isolate OC43, and variants thereof.

Severe acute respiratory syndrome coronavirus 2 (SARS-CoV-2) is the causative pathogen of the current coronavirus disease 2019 (COVID-19) pandemic. Despite the deployment of several effective SARS-CoV-2 vaccines, the pandemic has been sustained by the emergence of several variants of concern, including Beta (B.1.351), Delta (B.1.617.2), and Omicron (B.1.1.529), which display varying degrees of resistance to neutralizing antibodies acquired naturally or via vaccination^{1, 2, 3, 4, 5}. The clinical manifestations of primary SARS-CoV-2 infection can range in severity from asymptomatic or mild/moderate symptoms to respiratory failure, multiorgan dysfunction, and death^{6, 7, 8, 9, 10, 11}. The factors that determine the precise clinical outcome of infection are unclear, although age, gender, and comorbidities are known to contribute^{10, 12, 13, 14, 15, 16, 17}. However, little is known about how pre-existing or acquired T cell immunity influences the course of infection.

Recent studies have shown that SARS-CoV-2 infection or vaccination can elicit robust CD8⁺ and CD4⁺ T cell responses in humans'^{18, 19, 20, 21, 22, 23, 24, 25, 26}; however, previously unexposed individuals also have functional CD8⁺ and CD4⁺ T cells with reactivity to SARS-CoV-2^{27, 28, 29, 30}. This pre-existing T cell immunity is thought to result from prior exposure to one of 4 related human coronaviruses (HCoVs); OC43, HKU1, 229, and NL63, which are collectively responsible for about 15-30% of common cold infections in adult humans annually³¹. Approximately 50%-90% of the global population are seropositive for at least one common cold HCoV³². The HCoV family, which also includes 2 additional members that cause severe respiratory symptoms, SARS-CoV and Middle-Eastern respiratory syndrome coronavirus (MERS-CoV), share considerable genomic sequence identity, ranging from ˜86% between SARS-CoV-2 and SARS-CoV to ˜78% between SARS-CoV-2 and the other HCoVs³³ (Table 1). Given the high seropositivity rate of common cold HCoVs and the shared homology with SARS-CoV-2, it seems reasonable to assume that prior exposure to one or more of the common cold HCoVs is one source of pre-existing cross-reactive SARS-CoV-2 immunity in unexposed individuals.

TABLE 1
Whole-genome sequence homology between
SARS-CoV-2 (MT786327) and SARS-CoV, MERS-CoV,
OC43, HKU1, NL63, or 229E³³.

| | SARS- | MERS- | | | | |
|---|---|---|---|---|---|---|
| | CoV | CoV | OC43 | HKU1 | NL63 | 229E |
| | (NC_{—} | (NC_{—} | (NC_{—} | (NC_{—} | (NC_{—} | (NC_{—} |
| | 004718) | 019843) | 006213) | 006577) | 005831) | 002645) |
| SARS- | 86.85 | 81.25 | 79.34 | 81.58 | 80.09 | 78.40 |
| CoV-2 | | | | | | |

Pre-existing cross-reactive T cells have been associated with both protective and pathogenic immunity to SARS-CoV-2. Specifically, cross-reactive CD4⁺ T cells have been linked to enhanced immune responses against SARS-CoV-2 infection and vaccination^{34, 35} as well as to the development of severe COVID-19³⁶, whereas pre-existing cross-reactive CD8⁺ T cell responses have been correlated with reduced COVID-19 severity and shorter disease duration^{37, 38, 39}. The possibility that pre-existing immunity to SARS-CoV-2 might be derived from prior exposure to a related HCoV has important clinical implications.

For example, exposure to flaviviruses such as dengue and Zika viruses (and vaccination in the case of dengue virus) can either protect against subsequent infections with a different flavivirus or heterologous serotype or severely exacerbate them, leading to life-threatening complications and death^{40, 41, 42, 43, 44, 45, 46, 47, 48, 49, 50, 51, 52, 53}. Whether pre-existing anti-flaviviral immunity is protective or pathogenic depends on multiple variables, including the particular combination of flavivirus or serotype, the source of cross-reactive immunity (antibody, CD8⁺ T cells, CD4⁺ T cells), and the time between primary and subsequent infections^{54, 55, 56, 57, 58}. Thus, understanding the relationship between immunity elicited by common cold HCoVs and cross-reactivity to SARS-CoV-2 has implications not only for identifying factors responsible for the disparate clinical outcomes among COVID-19 patients, but also for the design of pan-coronavirus vaccines.

In this study, the inventors investigated the antigen cross-reactivity of pre-existing OC43-elicited CD8⁺ and CD4⁺ T cells and their biological roles during subsequent SARS-CoV-2 infection. Human leukocyte antigen (HLA) is a key determinant of the magnitude, breadth, and specificity of the T cell response in humans. To maintain human relevance, the inventors employed a transgenic HLA-B*0702 and HLA-DRB1*0101 Ifnar1^{-/-} mouse models. HLA-B*0702 and HLA-DRB1*0101 are 2 of the most common human MHC class I and II alleles and are expressed by up to 17.6% and 12.5%, respectively, among some populations^{59, 60}. Deletion of type I interferon receptors (Ifnar1^{-/-}) in these mice permits the study of immunity to viruses that are unable to replicate in wildtype mice with an intact IFN response. Interestingly, several human cohort studies have observed an association between severe COVID-19 and inborn or acquired deficiency in the type I IFN pathway^{61, 62, 63, 64, 65, 66, 67}. In one study, autoantibodies against type I IFNs were detected in 20% of patients with severe COVID-19, suggesting that such autoantibodies may be a common source of acquired immunodeficiency⁶¹. Additionally, HLA-B*0702 and HLA-DRB1*0101 Ifnar1^{-/-} mouse models were used to demonstrate that the CD8⁺ and CD4⁺ T cell responses to flaviviruses mirror the antigen specificities, immunodominance patterns, and kinetics of the T cell responses observed in infected humans, including the influence of pre-existing cross-reactive immunity in shaping the secondary T cell response and clinical outcomes^{57, 68, 69, 70, 71}. Thus, the HLA transgenic Ifnar1^{-/-} mice provide ideal models to directly address the question of whether prior exposure to common cold HCoVs can be a source of cross-reactive SARS-CoV-2 immunity in humans and, if so, how pre-existing cross-reactive immunity may influence the outcome of SARS-CoV-2 infection.

The inventors first identified human-relevant immunodominant SARS-CoV-2 CD8⁺ and CD4⁺ epitopes following immunization with DNA-based vaccines encoding SARS-CoV-2 spike (S), membrane (M), or nucleocapsid (N) proteins; following infection with mouse-adapted SARS-CoV-2 strain MA10⁷² or SARS-CoV-2 B.1.351 (isolate HCoV-19/South Africa/KRISP-K005325/2020); and following infection with OC43 virus. The inventors then established the cross-reactivity of OC43-elicited T cells to SARS-CoV-2 peptides, examined the effect of prior exposure to OC43 on subsequent SARS-CoV-2 infection and lung disease, and determined the contribution of cross-reactive CD8⁺ and CD4⁺ T cells to OC43-induced cross-protection. These results demonstrate for the first time that a single prior exposure to OC43 does indeed generate cross-protective immunity against SARS-CoV-2 infection and lung disease, and additionally that the protection is mediated, at least in part, by both CD8⁺ and CD4⁺ T cells.

Identification of SARS-CoV-2 epitopes recognized by T cells in DNA-vaccinated HLA-B*0702 and HLA-DRB1*0101 Ifnar1^{-/-} mice. To investigate CD8⁺ and CD4⁺ T cell responses to SARS-CoV-2 in the context of human HLA alleles, the major predicted HLA-B*0702- and HLA-DRB1*0101-restricted T cell epitopes in SARS-CoV-2 S, N, and M proteins were first identified, which are known to be major CD8⁺ and CD4⁺ T cell targets in infected humans²³. Using the Immune Epitope Database⁷³ to identify potentially immunogenic peptides, the inventors selected the top 1% of SARS-CoV-2 S, M, and N peptides predicted to have high-affinity binding to HLA-B*0702 or HLA-DRB1*010 and obtained 69 class I-restricted epitopes (Table 2) and 42 class II-restricted epitopes (Table 3).

TABLE 2
Predicted HLA-B*0702-restricted epitopes from
SARS-COV-2 S-, M-, and N-proteins.

| | | | | | SEQ |
|---|---|---|---|---|---|
| | | | | | ID |
| Protein | Start | End | Length | Sequence | NO: |
| Spike | 24 | 32 | 9 | LPPAYTNSF | 1 |
| Spike | 38 | 47 | 10 | YPDKVFRSSV | 2 |
| Spike | 38 | 48 | 11 | YPDKVFRSSVL | 3 |
| Spike | 56 | 65 | 10 | LPFFSNVTWF | 4 |
| Spike | 207 | 216 | 10 | HTPINLVRDL | 5 |
| Spike | 208 | 216 | 9 | TPINLVRDL | 6 |
| Spike | 216 | 223 | 8 | LPQGFSAL | 7 |
| Spike | 216 | 226 | 11 | LPQGFSALEPL | 8 |
| Spike | 329 | 338 | 10 | FPNITNLCPF | 9 |
| Spike | 411 | 419 | 9 | APGQTGKIA | 10 |
| Spike | 462 | 472 | 11 | KPFERDISTEI | 11 |
| Spike | 506 | 515 | 10 | QPYRVVVLSF | 12 |
| Spike | 506 | 513 | 8 | QPYRVVVL | 13 |
| Spike | 526 | 534 | 9 | GPKKSTNLV | 14 |
| Spike | 588 | 597 | 10 | TPCSFGGVSV | 15 |
| Spike | 620 | 629 | 10 | VPVAIHADQL | 16 |
| Spike | 630 | 638 | 9 | TPTWRVYST | 17 |
| Spike | 678 | 688 | 11 | TNSPRRARSVA | 18 |
| Spike | 679 | 688 | 10 | NSPRRARSVA | 19 |
| Spike | 680 | 688 | 9 | SPRRARSVA | 20 |
| Spike | 680 | 689 | 10 | SPRRARSVAS | 21 |
| Spike | 680 | 687 | 8 | SPRRARSV | 22 |
| Spike | 683 | 692 | 10 | RARSVASQSI | 23 |
| Spike | 691 | 699 | 9 | SIIAYTMSL | 24 |
| Spike | 713 | 722 | 10 | AIPTNFTISV | 25 |
| Spike | 714 | 722 | 9 | IPTNFTISV | 26 |
| Spike | 727 | 736 | 10 | LPVSMTKTSV | 27 |
| Spike | 811 | 821 | 11 | KPSKRSFIEDL | 28 |
| Spike | 811 | 818 | 8 | KPSKRSFI | 29 |
| Spike | 869 | 877 | 9 | MIAQYTSAL | 30 |
| Spike | 1014 | 1022 | 9 | RAAEIRASA | 31 |
| Spike | 1052 | 1061 | 10 | FPQSAPHGVV | 32 |
| Spike | 1052 | 1062 | 11 | FPQSAPHGVVF | 33 |
| Spike | 1052 | 1060 | 9 | FPQSAPHGV | 34 |
| Spike | 1056 | 1063 | 8 | APHGVVFL | 35 |
| Spike | 1089 | 1097 | 9 | FPREGVFVS | 36 |
| Spike | 1089 | 1096 | 8 | FPREGVFV | 37 |
| Spike | 1261 | 1270 | 10 | SEPVLKGVKL | 38 |
| Membrane | 58 | 67 | 10 | WPVTLACFVL | 39 |
| Membrane | 101 | 109 | 9 | RLFARTRSM | 40 |
| Membrane | 103 | 112 | 10 | FARTRSMWSF | 41 |
| Membrane | 122 | 129 | 8 | VPLHGTIL | 42 |
| Membrane | 131 | 138 | 8 | RPLLESEL | 43 |
| Membrane | 131 | 140 | 10 | RPLLESELVI | 44 |
| Membrane | 148 | 156 | 9 | HLRIAGHHL | 45 |
| Membrane | 164 | 172 | 9 | LPKEITVAT | 46 |
| Nucleo- | 12 | 21 | 10 | APRITFGGPS | 47 |
| protein | | | | | |
| Nucleo- | 41 | 50 | 10 | RPQGLPNNTA | 48 |
| protein | | | | | |
| Nucleo- | 45 | 53 | 9 | LPNNTASWF | 49 |
| protein | | | | | |
| Nucleo- | 64 | 74 | 11 | LKFPRGQGVPI | 50 |
| protein | | | | | |
| Nucleo- | 65 | 74 | 10 | KFPRGQGVPI | 51 |
| protein | | | | | |
| Nucleo- | 66 | 74 | 9 | FPRGQGVPI | 52 |
| protein | | | | | |
| Nucleo- | 66 | 75 | 10 | FPRGQGVPIN | 53 |
| protein | | | | | |
| Nucleo- | 66 | 76 | 11 | FPRGQGVPINT | 54 |
| protein | | | | | |
| Nucleo- | 66 | 73 | 8 | FPRGQGVP | 55 |
| protein | | | | | |
| Nucleo- | 67 | 74 | 8 | PRGQGVPI | 56 |
| protein | | | | | |
| Nucleo- | 93 | 101 | 9 | RIRGGDGKM | 57 |
| protein | | | | | |
| Nucleo- | 104 | 113 | 10 | LSPRWYFYYL | 58 |
| protein | | | | | |
| Nucleo- | 105 | 113 | 9 | SPRWYFYYL | 59 |
| protein | | | | | |
| Nucleo- | 105 | 114 | 10 | SPRWYFYYLG | 60 |
| protein | | | | | |
| Nucleo- | 149 | 158 | 10 | RNPANNAAIV | 61 |
| protein | | | | | |
| Nucleo- | 150 | 159 | 10 | NPANNAAIVL | 62 |
| protein | | | | | |
| Nucleo- | 150 | 158 | 9 | NPANNAAIV | 63 |
| protein | | | | | |
| Nucleo- | 161 | 171 | 11 | LPQGTTLPKGF | 64 |
| protein | | | | | |
| Nucleo- | 256 | 265 | 10 | KKPRQKRTAT | 65 |
| protein | | | | | |
| Nucleo- | 257 | 265 | 9 | KPRQKRTAT | 66 |
| protein | | | | | |
| Nucleo- | 257 | 267 | 11 | KPRQKRTATKA | 67 |
| protein | | | | | |
| Nucleo- | 257 | 264 | 8 | KPRQKRTA | 68 |
| protein | | | | | |
| Nucleo- | 308 | 317 | 10 | APSASAFFGM | 69 |
| protein | | | | | |

TABLE 3
Predicted HLA-DRB1*0101-restricted epitopes from
SARS-COV-2 S-, M-, and N-proteins.

| | | | | | SEQ |
|---|---|---|---|---|---|
| | | | | | ID |
| Protein | Start | End | Length | Sequence | NO: |
| Spike | 4 | 18 | 15 | FLVLLPLVSSQCVNL | 70 |
| Spike | 54 | 68 | 15 | LFLPFFSNVTWFHAI | 71 |
| Spike | 62 | 76 | 15 | VTWFHAIHVSGTNGT | 72 |
| Spike | 115 | 129 | 15 | QSLLIVNNATNVVIK | 73 |
| Spike | 199 | 213 | 15 | GYFKIYSKHTPINLV | 74 |
| Spike | 231 | 245 | 15 | IGINITRFQTLLALH | 75 |
| Spike | 236 | 250 | 15 | TRFQTLLALHRSYLT | 76 |
| Spike | 264 | 278 | 15 | AYYVGYLQPRTFLLK | 77 |
| Spike | 315 | 329 | 15 | TSNFRVQPTESIVRF | 78 |
| Spike | 344 | 358 | 15 | ATRFASVYAWNRKRI | 79 |
| Spike | 363 | 377 | 15 | ADYSVLYNSASFSTF | 80 |
| Spike | 512 | 526 | 15 | VLSFELLHAPATVCG | 81 |
| Spike | 539 | 553 | 15 | VNFNFNGLTGTGVLT | 82 |
| Spike | 692 | 706 | 15 | IIAYTMSLGAENSVA | 83 |
| Spike | 758 | 772 | 15 | SFCTQLNRALTGIAV | 84 |
| Spike | 853 | 867 | 15 | QKFNGLTVLPPLLTD | 85 |
| Spike | 869 | 883 | 15 | MIAQYTSALLAGTIT | 86 |
| Spike | 885 | 899 | 15 | GWTFGAGAALQIPFA | 87 |
| Spike | 895 | 909 | 15 | QIPFAMQMAYRENGI | 88 |
| Spike | 902 | 916 | 15 | MAYRFNGIGVTQNVL | 89 |
| Spike | 959 | 973 | 15 | LNTLVKQLSSNFGAI | 90 |
| Spike | 967 | 981 | 15 | SSNFGAISSVLNDIL | 91 |
| Spike | 998 | 1012 | 15 | TGRLQSLQTYVTQQL | 92 |
| Spike | 1005 | 1019 | 15 | QTYVTQQLIRAAEIR | 93 |
| Spike | 1010 | 1024 | 15 | QQLIRAAEIRASANL | 94 |
| Spike | 1015 | 1029 | 15 | AAEIRASANLAATKM | 95 |
| Spike | 1044 | 1058 | 15 | GKGYHLMSFPQSAPH | 96 |
| Membrane | 32 | 46 | 15 | ICLLQFAYANRNRFL | 97 |
| Membrane | 60 | 74 | 15 | VTLACFVLAAVYRIN | 98 |
| Membrane | 71 | 85 | 15 | YRINWITGGIAIAMA | 99 |
| Membrane | 91 | 105 | 15 | MWLSYFIASFRLFAR | 100 |
| Membrane | 98 | 112 | 15 | ASFRLFARTRSMWSF | 101 |
| Membrane | 116 | 130 | 15 | TNILLNVPLHGTILT | 102 |
| Membrane | 144 | 158 | 15 | ILRGHLRIAGHHLGR | 103 |
| Membrane | 165 | 179 | 15 | PKEITVATSRTLSYY | 104 |
| Membrane | 175 | 189 | 15 | TLSYYKLGASQRVAG | 105 |
| Nucleo- | 107 | 121 | 15 | RWYFYYLGTGPEAGL | 106 |
| protein | | | | | |
| Nucleo- | 129 | 143 | 15 | GIIWVATEGALNTPK | 107 |
| protein | | | | | |
| Nucleo- | 154 | 168 | 15 | NAAIVLQLPQGTTLP | 108 |
| protein | | | | | |
| Nucleo- | 303 | 317 | 15 | QIAQFAPSASAFFGM | 109 |
| protein | | | | | |
| Nucleo- | 328 | 342 | 15 | GTWLTYTGAIKLDDK | 110 |
| protein | | | | | |
| Nucleo- | 387 | 401 | 15 | KKQQTVTLLPAADLD | 111 |
| protein | | | | | |

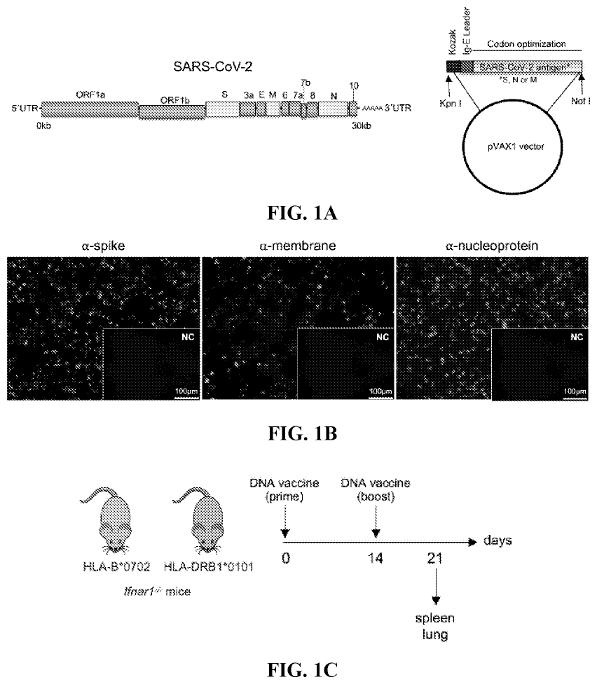

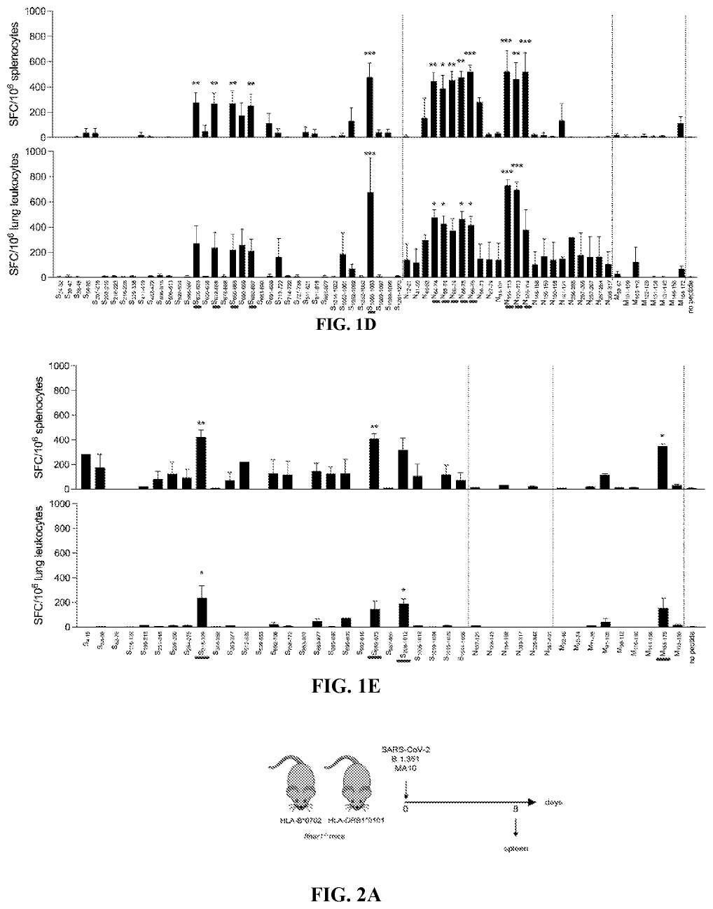

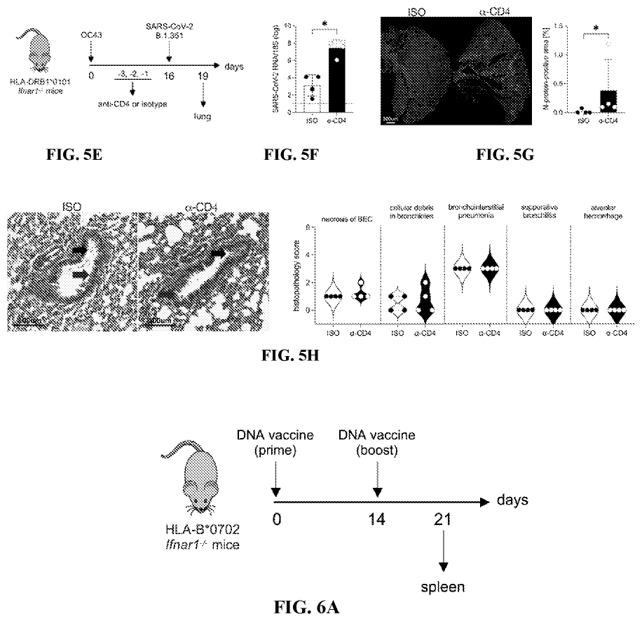

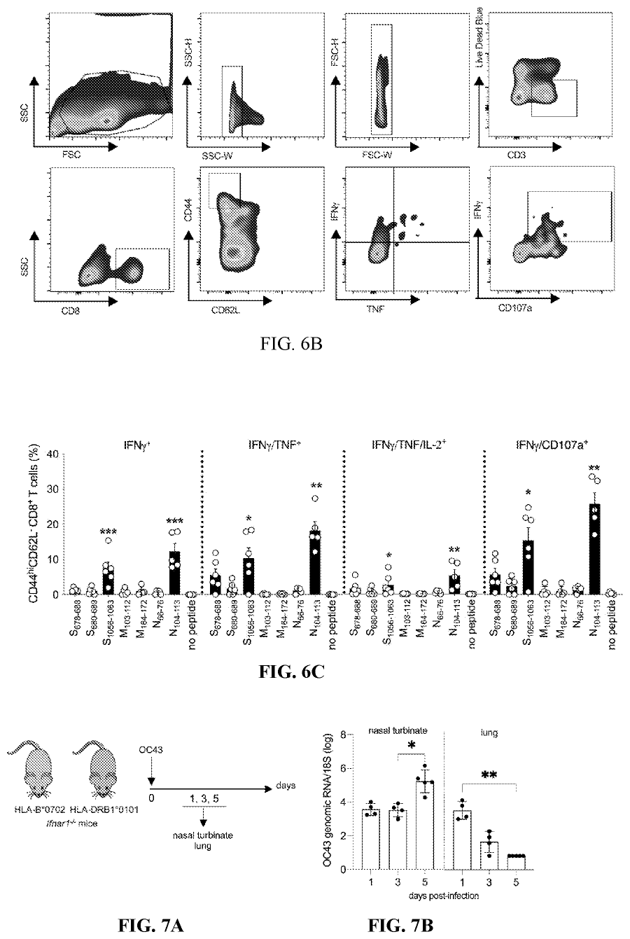

Mice from both strains were vaccinated with a DNA-based vaccine encoding SARS-CoV-2 S, M, or N proteins (FIGS. 1A and 1B) on days 0 and 14, and spleens and lungs were collected 7 days later (FIG. 1C). Splenocytes or lung leukocytes were incubated with each peptide (vs no peptide control), and IFNγ-producing peptide-specific T cells quantified using ELISpot assays. Splenocytes from DNA-vaccinated HLA-B*0702 transgenic mice produced significantly higher levels of IFNγ in response to 13 of the 69 peptides (S₆₂₀₋₆₂₉, S₆₇₈₋₆₈₈, S₆₈₀₋₆₈₇, S₆₈₀₋₆₈₈, S₁₀₅₀₋₁₀₆₃, N₆₄₋₇₄, N₆₅₋₇₄, N₆₆₋₇₄, N₆₆₋₇₅, N₆₆₋₇₆, N₁₀₄₋₁₁₃, N₁₀₅₋₁₁₃, and N₁₀₅₋₁₁₄) compared with unstimulated control cells, whereas lung leukocytes from HLA-B*0702 mice showed significant IFNγ secretion in response to 7 of the 69 peptides (S₁₀₅₆₋₁₀₆₃, N₆₄₋₇₄, N₆₅₋₇₄, N₆₆₋₇₅, N₆₆₋₇₆, N₁₀₄₋₁₁₃, and N₁₀₅₋₁₁₃; FIG. 1D). Of the 7 peptides that induced significant IFNγ responses in both lung leukocytes and splenocytes, the highest response was to the 3 peptides spanning residues 104 to 113 of the N protein (FIG. 1D), and this was confirmed by intracellular cytokine staining (ICS) (FIG. 6A to 6C). In vaccinated HLA-DRB1*0101 mice, 3 of the 42 predicted peptides (S₃₁₅₋₃₂₉, S₉₅₉₋₉₇₃, and M₁₆₅₋₁₇₉) resulted in significant stimulation of IFNγ production by splenocytes, 2 peptides (5315-329 and S₉₉₈₋₁₀₁₂) significantly stimulated a response in lung leukocytes, and S₃₁₅₋₃₂₉ was capable of stimulating both splenocytes and lung leukocytes (FIG. 1E). In contrast to the class I-restricted response, none of the N protein-derived peptides stimulated a significant IFNγ response in DNA-vaccinated HLA-DRB1*0101 mice. These data demonstrate that SARS-CoV-2 DNA-based vaccines elicited T cell responses dominated by recognition of S and N protein-derived peptides in the spleen and lung of HLA-B*0702Ifnar1^{-/-} mice and by S and M protein-derived peptides in HLA-DRB1*0101 Ifnar1^{-/-} mice.

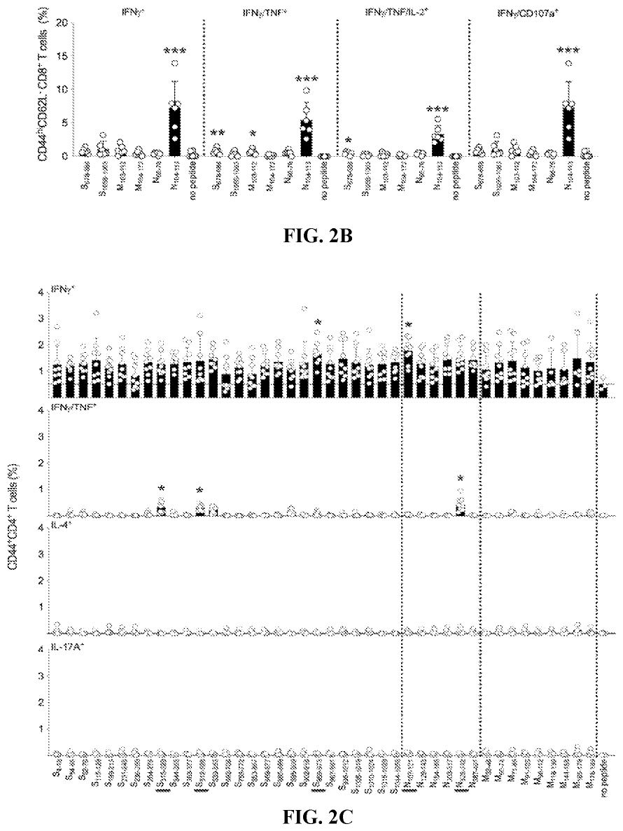

SARS-CoV-2 infection elicits effector CD8⁺ and Th1-biased CD4⁺ T cell responses in HLA transgenic Ifnar1^{-/-} mice. The inventors next determined whether the antigen-specificities of the T cell response elicited by SARS-CoV-2 DNA vaccines were similar to those induced by live virus. To this end, the inventors infected HLA-DRB1*0101 Ifnar1^{-/-} mice with mouse-adapted SARS-CoV-2 MA10 strain⁷² and HLA-B*0702 Ifnar1^{-/-} mice with SARS-CoV-2 B.1.351 (Beta), which can replicate in mice without the need for adaptation^{74, 75}. Spleens were collected on day 8 post-infection (FIG. 2A), splenocytes stimulated with selected SARS-CoV-2 peptides, and immunolabeled for cell surface markers, intracellular cytokines, and the degranulation marker CD107a, and the frequency of activated (CD44⁺ CD62L⁻) effector CD8⁺ and CD4⁺ T cells quantified by flow cytometry.

The CD8⁺ T cell response in B.1.351-infected HLA-B*0702 Ifnar1^{-/-} mice was assessed by stimulating splenocytes with the 6 most potent SARS-CoV-2-derived peptides identified by DNA vaccination (S₆₇₈₋₆₈₈, S₁₀₅₆₋₁₀₆₃, N₆₆₋₇₆, N₁₀₄₋₁₁₃, M₁₀₃₋₁₁₂, M₁₆₄₋₁₇₂). The frequencies of activated IFNγ⁺, IFNγ⁺/TNF⁺, IFNγ⁺/CD107a⁺, and IFNγ+/TNF⁺/IL-2⁺ CD8⁺ T cells were significantly increased in response to stimulation with epitope N₁₀₄₋₁₁₃ compared with the unstimulated cultures, whereas no significant expansion was observed in response to the other peptides (FIG. 2B). Thus, both vaccination with a SARS-CoV-2 N protein-encoding DNA vaccine and direct infection with B.1.351 elicited an effector CD8⁺ T cell response in HLA-B*0702 Ifnar1^{-/-} mice that was directed against the immunodominant epitope N₁₀₄₋₁₁₃. This finding is in agreement with previous reports that SARS-CoV-2 N₁₀₅₋₁₁₃ is the immunodominant epitope in SARS-CoV-2-infected individuals expressing HLA-B*0702^{25, 28, 76, 77, 78, 79}.

As the class II-restricted response to SARS-CoV-2 DNA vaccines was lower in magnitude than the class I-restricted response, the inventors stimulated splenocytes from MA10-infected HLA-DRB1*0101 Ifnar1^{-/-} mice with each of the 42 SARS-CoV-2 peptides predicted to be immunogenic in the context of HLA-DRB1*0101, and then analyzed the frequencies of activated CD4⁺ T cells producing IFNγ alone or IFNγ and TNF (Th1 cells), IL-4 (Th2 cells), and IL-17A (Th17 cells) (FIG. 2C). All 42 peptides increased the frequency of IFNγ-producing cells compared with unstimulated control cells, but the increase was significant only in response to 2 peptides; S₉₅₉₋₉₇₃ and N₁₀₇₋₁₂₁. Three peptides were capable of expanding multifunctional IFNγ⁺/TNF⁺CD4⁺ T cells (S₃₁₅₋₃₂₉, S₅₁₂₋₅₂₆, and N₃₂₈₋₃₄₂), whereas the frequency of CD4⁺ T cells producing IL-4 or IL-17A was not significantly increased by any of the peptides evaluated. Of note, N₁₀₇₋₁₂₁ largely encompasses the immunodominant N₁₀₄₋₁₁₃ CD8⁺ T cell epitope identified in both the DNA-vaccinated and SARS-CoV-2-infected HLA-B*0702 Ifnar1^{-/-} mice, and S₃₁₅₋₃₂₉ also stimulated splenocytes from the DNA-vaccinated HLA-DRB1*0101 Ifnar1^{-/-} mice. These findings demonstrate that the major CD4⁺ T cell response to primary infection with SARS-CoV-2 MA10 in HLA-DRB1*0101 Ifnar1^{-/-} mice is Th1-biased, which is consistent with studies showing that SARS-CoV-2 infection or vaccination of humans elicits CD4⁺ T cells with a Th1-like phenotype^{23, 80, 81}.

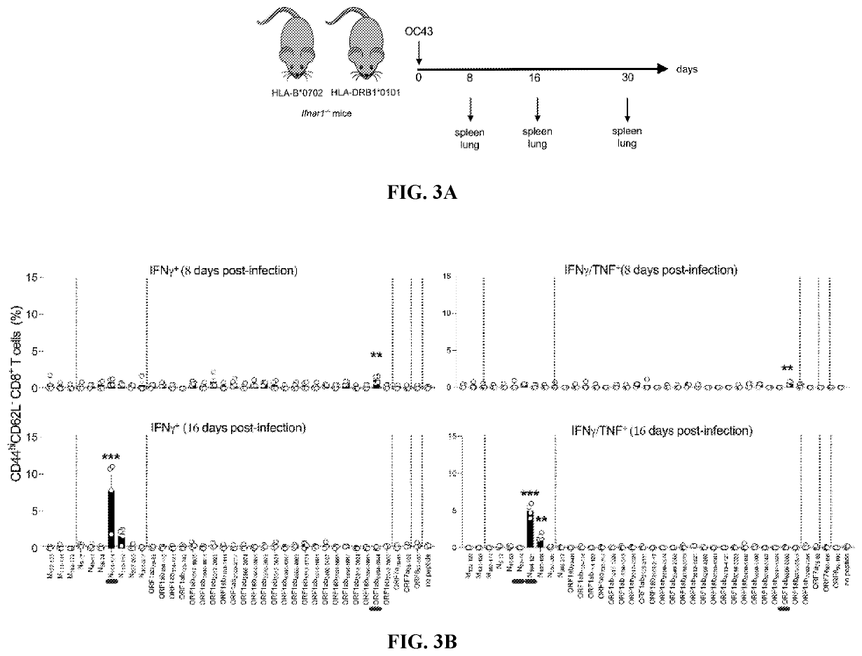

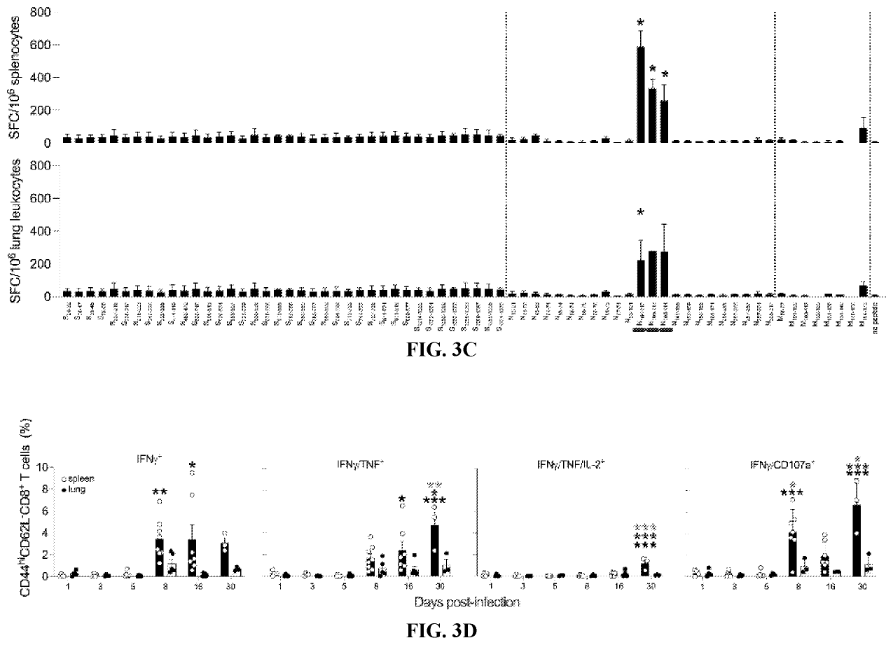

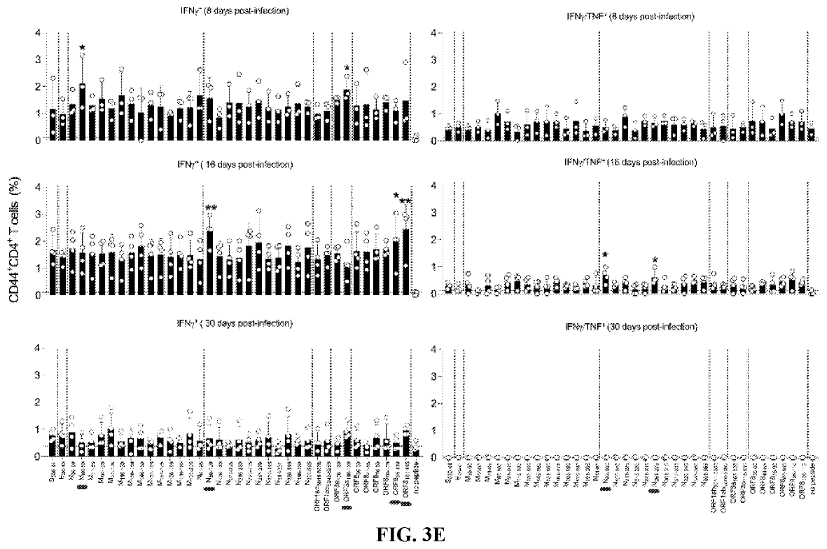

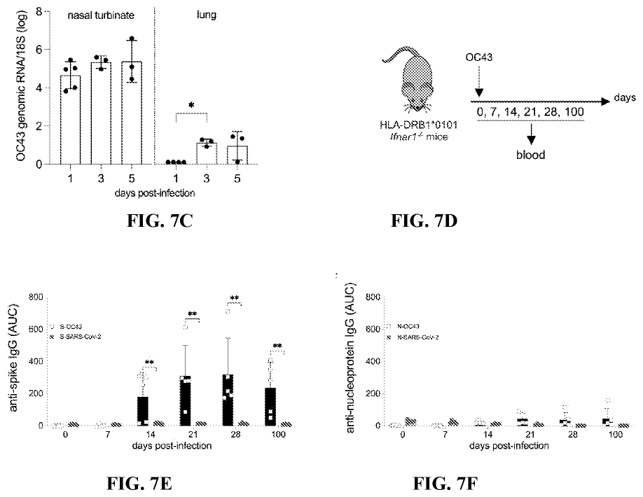

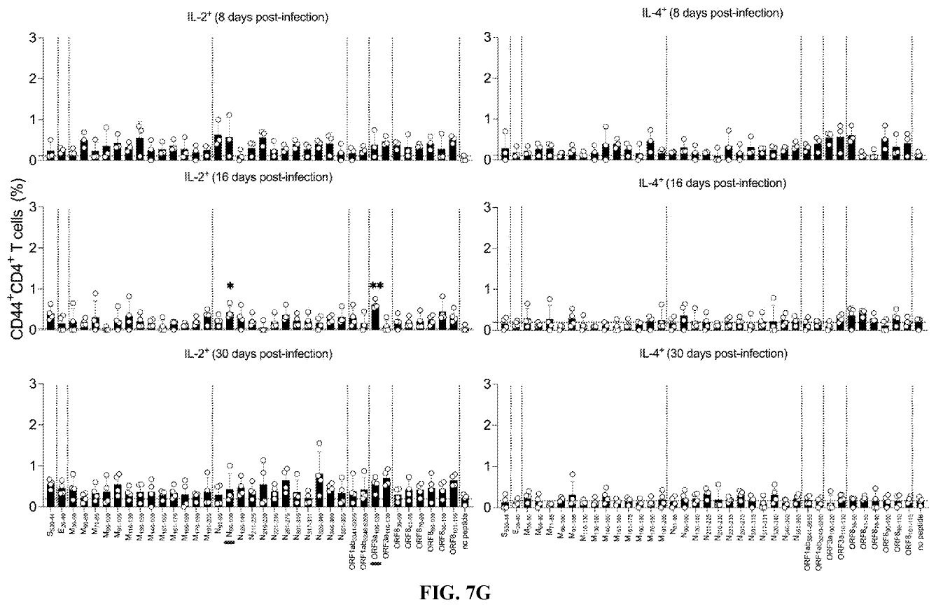

OC43 infection elicits CD8⁺ T cells with cross-reactivity to SARS-CoV-2 in HLA-B*0702 Ifnar1^{-/-} mice. The genomic sequence of SARS-CoV-2 N protein is 29% and 23% identical to the N protein sequences of β-coronaviruses (OC43 and HKU-1) and α-coronaviruses (NL63 and 229E), respectively^{82, 83}. To investigate whether exposure to seasonal common cold HCoVs can elicit CD8⁺ T cells that cross-react with SARS-CoV-2, the inventors infected HLA-B*0702 Ifnar1^{-/-} mice with OC43, the most common seasonal HCoV worldwide^{84, 85}. The viral load in upper and lower airway tissues on days 1, 3, and 5 post-infection was analyzed (FIG. 7A), and CD8⁺ T cell responses to SARS-CoV-2 peptides on days 8 and 16 post-infection (FIG. 3A). While OC43 genomic RNA levels in nasal turbinates increased between days 1 and 5, levels in lung were highest on day 1 and below the level of detection by day 5 (FIG. 7B). Splenocytes prepared on days 8 and 16 post-infection were stimulated with a panel of 37 HLA-1B*0702-restricted SARS-CoV-2 CD8⁺ T cell epitopes that have previously been demonstrated to stimulate human CD8⁺ T cells based on IFNγ-ELISpot or ICS assays (NIAID Virus Pathogen Database and Analysis Resource; Table 4)^{25, 86, 87, 88, 89, 90, 91, 92}. This panel included peptides from SARS-CoV-2 M (n=3), N (n=7), ORF1ab (n=24), ORF7 (n=2), and ORF8 (n=1) proteins, and was selected to ensure that the analysis of OC43-elicited T cell reactivity was focused on the most human-relevant SARS-CoV-2 epitopes. At day 8 post-OC43 infection, the frequencies of activated IFNγ⁺ and IFNγ/TNF⁺CD8⁺ T cells were increased in response to several SARS-CoV-2 peptides, but the increase was significant only for ORF1ab₆₈₃₄₋₆₈₄₄ (FIG. 3B). At day 16 post-infection, the activated CD8+ T cell response focused to a single region in the N protein, with 9- and 12-fold expansion of N₁₀₄₋₁₂₁-reactive IFNγ⁺ and IFNγ⁺/TNF⁺ CD8⁺ T cells, respectively (FIG. 3B). To validate this finding, splenocytes and lung leukocytes were isolated from HLA-1B*0702 Ifnar1^{-/-} mice on day 8 post-OC43 infection and stimulated with the 69-peptide panel (Table 2) previously examined with cells from DNA-vaccinated and SARS-CoV-2-infected mice. Indeed, the frequencies of IFNγ-producing splenocytes and lung leukocytes were increased by only 3 SARS-CoV-2 peptides, all encompassing the N₁₀₄₋₁₁₃ epitope (FIG. 3C). Thus, exposure to OC43 elicits HLA-B*0702-restricted CD8⁺ T cells with reactivity to the SARS-CoV-2 N₁₀₄₋₁₁₃ epitope. Of note, the amino acid sequences of the SARS-CoV-2 and OC43 N₁₀₄₋₁₁₃ sequence differs by only a single residue (LSPRWYFYYL and LLPRWYFYYL, respectively), providing a basis for this cross-reactivity.

TABLE 4
HLA-B*0702-restricted CD8+ T cell epitopes
identified

| | | | | | SEQ ID |
|---|---|---|---|---|---|
| Protein | Start | End | Length | Sequence | NO: |
| Membrane | 122 | 129 | 8 | VPLHGTIL | 112 |
| Membrane | 131 | 138 | 8 | RPLLESEL | 113 |
| Membrane | 164 | 172 | 9 | LPKEITVAT | 114 |
| Nucleocapsid | 5 | 17 | 13 | GPQNQRNAPRITF | 115 |
| Nucleocapsid | 45 | 53 | 9 | LPNNTASWF | 116 |
| Nucleocapsid | 66 | 74 | 9 | FPRGQGVPI | 117 |
| Nucleocapsid | 104 | 121 | 18 | LSPRWYFYYLGTG | 118 |
| | | | | PEAGL | |
| Nucleocapsid | 150 | 159 | 10 | NPANNAAIVL | 119 |
| Nucleocapsid | 257 | 265 | 9 | KPRQKRTAT | 120 |
| Nucleocapsid | 308 | 317 | 10 | APSASAFFGM | 121 |
| ORF1ab | 79 | 88 | 10 | APHGHVMVEL | 122 |
| ORF1ab | 108 | 116 | 9 | VPHVGEIPV | 123 |
| ORF1ab | 114 | 123 | 10 | IPVAYRKVLL | 124 |
| ORF1ab | 735 | 742 | 8 | APKEIIFL | 125 |
| ORF1ab | 1017 | 1025 | 9 | TPVVQTIEV | 126 |
| ORF1ab | 1608 | 1618 | 11 | KPHNSHEGKTF | 127 |
| ORF1ab | 2017 | 2028 | 12 | KPVETSNSFDVL | 128 |
| ORF1ab | 2109 | 2117 | 9 | KPNELSRVL | 129 |
| ORF1ab | 2703 | 2711 | 9 | VAKSHNIAL | 130 |
| ORF1ab | 2866 | 2874 | 9 | VPGLPGTIL | 131 |
| ORF1ab | 2949 | 2956 | 8 | RPDTRYVL | 132 |
| ORF1ab | 3370 | 3378 | 9 | QPGQTFSVL | 133 |
| ORF1ab | 3612 | 3621 | 10 | LPFAMGIIAM | 134 |
| ORF1ab | 4260 | 4267 | 8 | VPANSTVL | 135 |
| ORF1ab | 4655 | 4663 | 9 | KPYIKWDLL | 136 |
| ORF1ab | 4713 | 4721 | 9 | FPPTSFGPL | 137 |
| ORF1ab | 5018 | 5028 | 11 | MPNMLRIMASL | 138 |
| ORF1ab | 5400 | 5407 | 8 | KPPISFPL | 139 |
| ORF1ab | 5658 | 5666 | 9 | IPARARVEC | 140 |
| ORF1ab | 5658 | 5667 | 10 | IPARARVECF | 141 |
| ORF1ab | 5914 | 5924 | 11 | LEIPRRNVATL | 142 |
| ORF1ab | 6656 | 6665 | 10 | KPRSQMEIDF | 143 |
| ORF1ab | 6834 | 6844 | 11 | LPKGIMMNV | 144 |
| ORF1ab | 7048 | 7056 | 9 | FPLKLRGTA | 145 |
| ORF7 | 78 | 86 | 9 | RARSVSPKL | 146 |
| ORF7 | 98 | 106 | 9 | SPIFLIVAA | 147 |
| ORF8 | 92 | 100 | 9 | EPKLGSLVV | 148 |

The inventors extended this investigation by following the development of the SARS-CoV-2 N₁₀₄₋₁₁₃-reactive effector CD8⁺ T cell response in spleen and lungs for 30 days following OC43 infection (FIGS. 3A and 3D). Expansion of N₁₀₄₋₁₁₃-reactive IFN and IFNγ⁺/TNF⁺ CD8⁺ T cells was evident by day 8 after OC43 infection and remained stable (IFNγ⁺) or gradually increased (IFNγ⁺/TNF⁺) up to the end of the analysis (day 30). In contrast, polyfunctional IFNγ⁺/TNF⁺/IL-2⁺ cells were undetectable until day 30, at which point a small but significant expansion of SARS-CoV-2 N₁₀₄₄₋₁₁₃-reactive cells was detected in spleen but not lung. IFNγ⁺/CD107a⁺CD8⁺ T cells exhibited a biphasic response that was detectable by day 8, waned between days 8 and 16, and increased again by day 30. These data provide the first direct demonstration that the seasonal coronavirus OC43 elicits CD8⁺ T cells that cross-react with SARS-CoV-2, and additionally demonstrates that this cross-reactive response is directed against a single immunodominant SARS-CoV-2 epitope, N₁₀₄₋₁₁₃, in the context of HLA-1B*0702.

OC43 infection elicits CD4⁺ T cells with cross-reactivity to SARS-CoV-2. To determine whether OC43 infection can also induce a CD4⁺ T cell response that cross-reacts with SARS-CoV-2, HLA-DRB1*0101 Ifnar1^{-/-} mice were infected with OC43, followed by analysis of viral load from days 1 to 5 (FIG. 7A) and CD4⁺ T cell response from days 0 to 30 post-infection (FIG. 3A). Levels of OC43 genomic RNA were high in nasal turbinates on all 3 days, and dramatically lower in lung (undetectable on day 1 and rising significantly but only slightly on day 3) (FIG. 7C). Splenocytes were stimulated with a panel of 37 HLA-DRB1*0101-restricted peptides derived from SARS-CoV-2 E, S, M, N, ORF1ab, ORF3a, and ORF8 proteins (Table 4) that had previously been shown to stimulate human CD4⁺ T cell responses by IFNγ-ELISpot, ICS, or MHC-binding assays^{18, 87, 93, 94, 95, 96, 97} and analyzed at days 8, 16, and 30 (FIG. 3E). At day 8, IFNγ⁺CD4⁺ T cells reactive with all 37 peptides were expanded in the spleen, although the increase was statistically significant only for cells stimulated with M₆₆₋₈₀ and ORF3a₁₁₆₋₁₃₀. At day 16, frequencies of IFNγ⁺ CD4⁺ T cells cross-reactive with SARS-CoV-2 ORF8₉₆₋₁₁₀ and ORF8₁₀₁₋₁₁₅ were significantly increased. In contrast, polyfunctional SARS-CoV-2 cross-reactive IFNγ⁺/TNF⁺CD4⁺ T cells were significantly expanded only in response to N₈₆₋₁₀₀, and N₂₆₁₋₂₇₅ peptides. In addition, while N₈₆₋₁₀₀ and ORF3a₁₀₈₋₁₂₀ peptides stimulated IL-2 production at day 16, none of the 37 peptides stimulated IL-4 expression (FIG. 7G). Finally, at day 30, the CD4⁺ T cell response was decreased for all peptides. Thus, exposure to OC43 elicits a Th1-biased CD4⁺ T cell response that cross-reacts with SARS-CoV-2 in the context of HLA-DRB1*0101.

Next it was determined whether OC43 infection stimulated a SARS-CoV-2 cross-reactive antibody response in the context of HLA-DRB1*0101, given that CD4⁺ T cells play crucial roles in promoting and maintaining antibody responses^{98, 99}. Sera isolated from mice at multiple time points between 0 and 100 days post-infection (FIG. 7D) was analyzed by ELISA for the presence of antibodies specific for OC43 or SARS-CoV-2 S and N proteins. Anti-OC43 S IgG titers were detectable by day 14 post-OC43 infection and remained relatively stable up to day 100 (the last day assessed); in contrast, IgG reactive with SARS-CoV-2 S protein was not detected in OC43-infected mice at any time point (FIG. 7E). IgG titers against N protein of both OC43 and SARS-CoV-2 were minimal at all time points (FIG. 7F). Thus, in this model of primary OC43 infection in HLA-DRB1*0101 Ifnar1^{-/-} mice, OC43 S-specific IgG and SARS-CoV-2-reactive CD4⁺ T cells are present. These data demonstrate that prior exposure to OC43 elicits an HLA-DRB1*0101-restricted CD4⁺ T cell response that cross-reacts with SARS-CoV-2 epitopes.

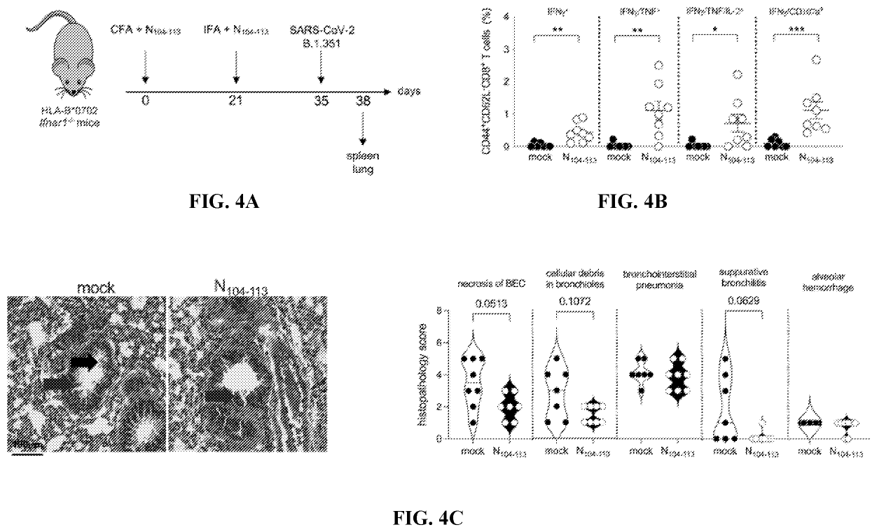

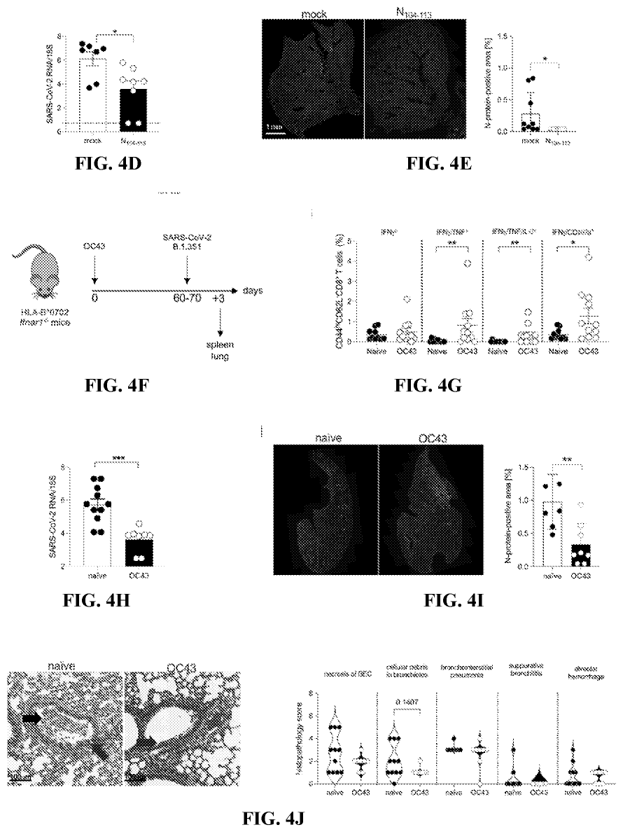

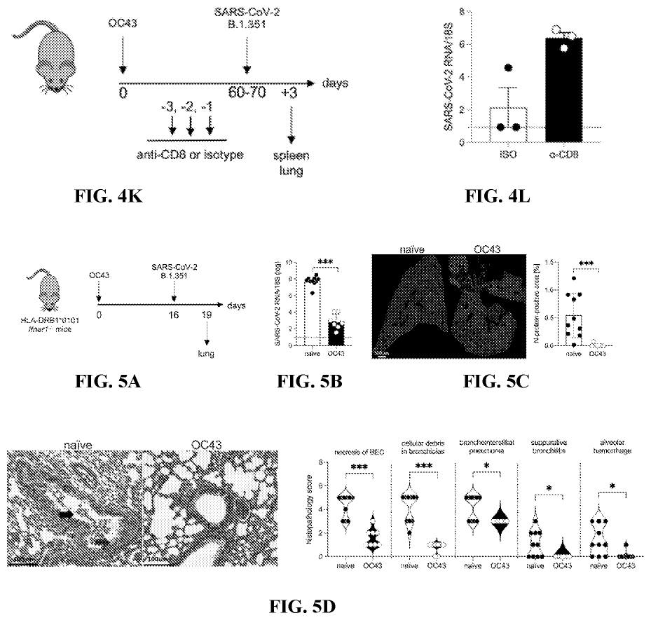

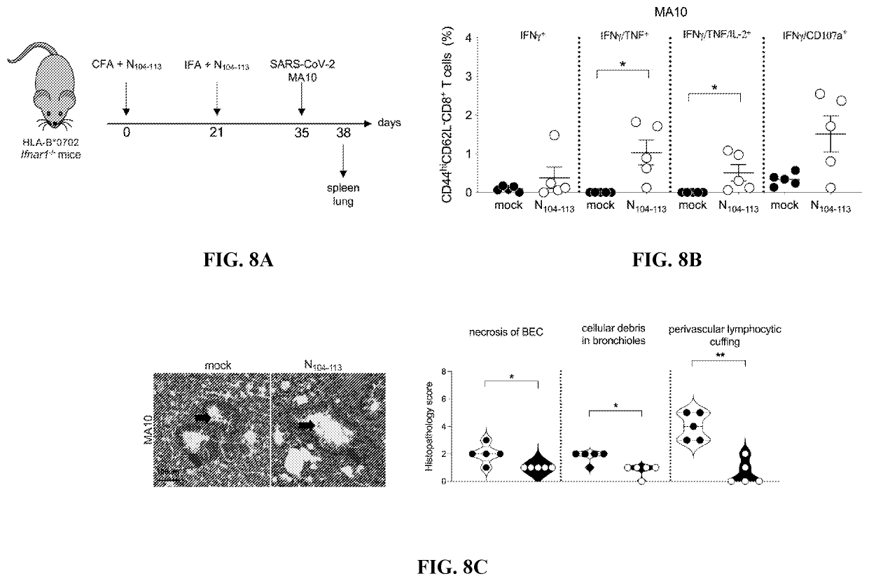

Immunization with N₁₀₄₋₁₁₃ peptide confers protection against SARS-CoV-2 infection and lung damage in HLA-B*0702 Ifnar1^{-/-} mice. To determine whether the OC43 cross-reactive CD8⁺ T cell response can protect against or exacerbate SARS-CoV-2 infection and/or pathology, HLA-B*0702 Ifnar1^{-/-} mice were primed and boosted with N₁₀₄₋₁₁₃ peptide on days 0 and 21, challenged with SARS-CoV-2 B.1.351 at 14 days post-boost, and tissues harvested on day 3 post-challenge (FIG. 4A). The frequencies of N₁₀₄₋₁₁₃-reactive polyfunctional (IFNγ⁺/TNF⁺ and IFNγ⁺/TNF⁺/IL-2⁺) and cytotoxic multifunctional (IFNγ⁺/CD107a⁺) CD8⁺ T cells were significantly increased in N₁₀₄₋₁₁₃-immunized mice (vs mock-immunized) (FIG. 4B). Histopathological analysis revealed that lungs from N₁₀₄₋₁₁₃-immunized mice appeared healthier (FIG. 4C, left panel). Quantification of specific lung histopathology criteria¹⁰⁰ revealed 3 features of SARS-CoV-2-induced lung damage that were less pronounced (lower scores) in N_{104/113}-immunized mice, although these differences were not significant: necrosis of bronchiolar epithelial cells (BEC), cellular debris in bronchioles, and suppurative bronchiolitis. This result was confirmed in mice challenged with the MA10 strain of SARS-CoV-2 (FIG. 8A; MA10 causes more severe lung disease than B.1.351), which revealed that the N₁₀₄₋₁₁₃-immunized (vs mock-immunized) mice had significantly higher frequencies of N₁₀₄₋₁₁₃-reactive polyfunctional CD8⁺ T cells (FIG. 8B) and significantly lower histopathology scores (FIG. 8C). For the B.1.351-challenged mice, the viral burden in lung was analyzed.

Both RT-qPCR analysis of genomic RNA (FIG. 4D) and immunofluorescence analysis of SARS-CoV-2 N protein (FIG. 4E) revealed significantly lower levels of SARS-CoV-2 in lungs of the N_{104/113}-immunized mice. These results demonstrate that immunization of HLA-B*0702Ifnar1^{-/-} mice with SARS-CoV-2 N₁₀₄₋₁₁₃ peptide elicits an antigen-specific polyfunctional CD8⁺ T cell response and protects against SARS-CoV-2 infection and lung disease.

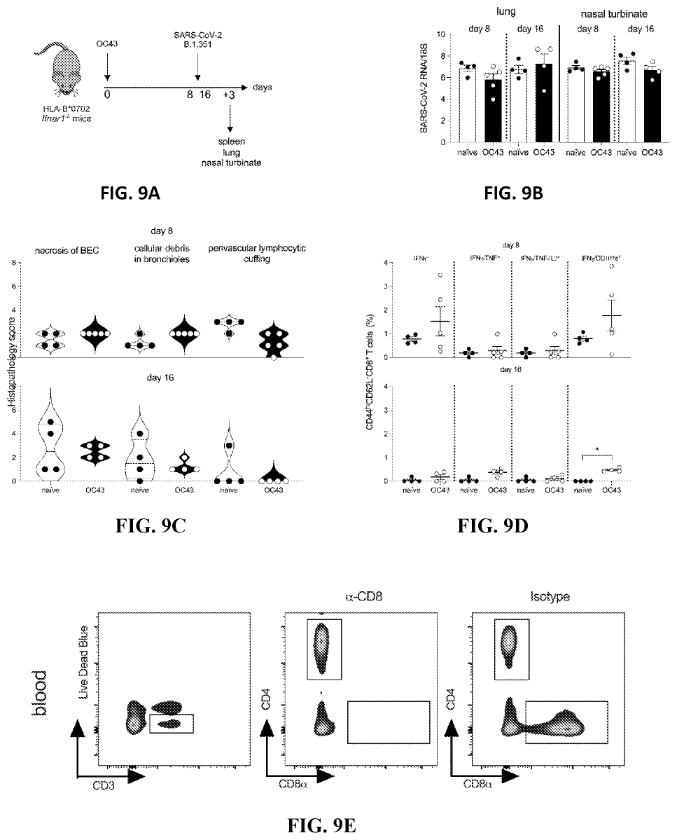

Prior exposure to OC43 confers cross-protection against SARS-CoV-2 infection and lung damage in HLA-B*0702 Ifnar1^{-/-} mice. Given that N₁₀₄₋₁₁₃-immunized mice exhibited reduced SARS-CoV-2 burden and pathogenesis than mock-immunized mice, it was hypothesized that OC43-elicited CD8⁺ T cell immunity might also provide protection against SARS-CoV-2 infection and lung damage. To test this, HLA-B*0702 Ifnar^{-/-} mice were infected with OC43 and challenged with SARS-CoV-2 on day 8 or 16 post-infection (FIG. 9A), or 60-70 days post-infection (FIG. 4F). Virologic and immunologic phenotypes were analyzed at 3 days post-challenge, which allowed a focus on the effects of OC43-elicited immunity-rather than the primary T cell response to SARS-CoV-2 (primary antiviral T cell responses are generally not detectable until days 4 or 5 post-infection^{44, 45, 71}).

RT-qPCR analysis revealed no effect of OC43 pre-exposure on SARS-CoV-2 genomic RNA levels in either lungs or nasal turbinates of mice challenged on days 8 or 16 (FIG. 9B). In contrast, lungs from mice challenged 60 to 70 days post-OC43 infection exhibited dramatic reductions in both SARS-CoV-2 genomic RNA (FIG. 4H) and N-protein immunoreactivity (FIG. 4I). While blinded histopathological analysis of lungs revealed no differences between OC43-infected and naïve mice challenged at 8 or 16 days post-infection (FIG. 9C), lungs of mice challenged at 60 to 70 days tended to have more bronchioles with clear lumina and viable epithelial cells lining the airway (i.e., proper polarization) (FIG. 4J, left panel), and exhibited decreases in 3 histopathologic features: necrotic epithelial cells, cellular debris within bronchioles, and bronchiolar lesions (FIG. 4J, right panel). However, these differences at 60 to 70 days post-infection were not significant. Thus, a single prior IN exposure to OC43 can protect against SARS-CoV-2 infection in HLA-B*0702 Ifnar1^{-/-} mice, and may also limit SARS-CoV-2-induced lung damage in some mice.

To test for association between OC43-induced protection and the OC43-elicited cross-reactive CD8⁺ T cell response in HLA-B*0702Ifnar1^{-/-} mice, splenocytes from OC-43-exposed (vs naïve) SARS-CoV-2-challenged mice were stimulated with N₁₀₄₋₁₁₃ peptide and analyzed by ICS (FIG. 4G). The frequencies of polyfunctional CD8⁺ T cells were significantly increased in mice challenged at 60-70 days post-OC43 infection (FIG. 4G). In contrast, in mice challenged at 8 or 16 days there was some expansion of activated effector CD8⁺ T cell subsets, but these increases were generally not significant (FIG. 9D). These results suggested that CD8⁺ T cells elicited by OC43 pre-exposure for 60-70 days might provide cross-protection during SARS-CoV-2 infection. To test this, mice were again infected with OC43 and challenged at 60 to 70 days, but also treated the mice with a depleting anti-CD8 antibody (vs isotype control) immediately prior to challenge (FIG. 4K). Efficient depletion of CD8⁺ T cells was confirmed by flow cytometry of blood and spleen (FIG. 9E). Notably, CD8⁺ T cell depletion abrogated the reduction in SARS-CoV-2 genomic RNA in lungs of mice with prior OC43 infection (FIG. 4L). These data indicate that prior exposure to OC43 elicits SARS-CoV-2-cross-reactive CD8⁺ T cells that contribute to the cross-protection against SARS-CoV-2 infection.

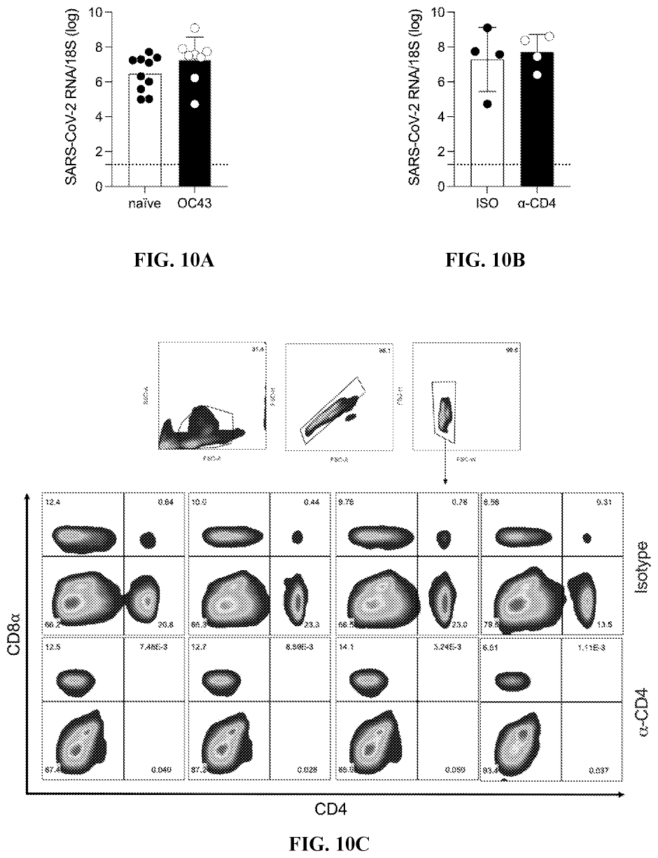

Prior exposure to OC43 infection confers cross-protection against SARS-CoV-2 infection and lung disease in HLA-DRB1*0101 Ifnar1^{-/-} mice in a manner partially dependent on CD4⁺ T cells. It was shown hereinabove that HLA-DRB1*0101 Ifnar1^{-/-} mice, like HLA-B*0702 Ifnar1^{-/-} mice, mount an antigen-specific CD4⁺ T cell response against SARS-CoV-2 after DNA vaccination or viral infection (FIGS. 1E and 2C), and a CD4⁺ T cell response to OC43 that cross-reacts with SARS-CoV-2 (FIGS. 3E and 7G). To determine whether OC43 pre-exposure can protect against SARS-CoV-2 infection, HLA-DRB1*0101 Ifnar1^{-/-} mice were infected with OC43, challenged with SARS-CoV-2 at 16 days post-infection (SARS-CoV-2 cross-reactive Th1 CD4⁺ T cell response peak at 16 days post-OC43 infection; FIG. 3E), and lungs harvested 3 days later (FIG. 5A). RT-qPCR analysis of SARS-CoV-2 genomic RNA and immunofluorescence staining of N protein revealed dramatically lower levels of SARS-CoV-2 infection in lungs from OC43-exposed (vs naïve) mice (FIGS. 5B and 5C). In contrast, prior OC43 exposure had no significant effect on SARS-CoV-2 RNA levels in nasal turbinates (FIG. 10A). Histopathological analysis showed that the lungs of OC43-exposed/SARS-CoV-2-challenged mice were much healthier than those of naïve/SARS-CoV-2-challenged mice. Specifically, lungs of OC43-exposed mice had more bronchioles with clear lumina and viable epithelial cells, and showed significant improvement in all 5 histopathological features quantified (FIG. 5D). Thus, OC43 pre-exposure for 16 days was sufficient to confer cross-protection against SARS-CoV-2 infection and lung disease in HLA-DRB1*0101 Ifnar1^{-/-} mice.

To determine whether these protective effects of OC43 pre-exposure were mediated by CD4⁺ T cells, these experiments were repeated in mice that were treated with a CD4 T cell-depleting antibody (vs isotype) immediately prior to SARS-CoV-2 challenge (FIG. 5E). Efficient depletion of CD4⁺ T cells was confirmed by flow cytometry (FIG. 10C). It was found that CD4⁺ T cell depletion significantly reduced the protective effect of prior OC43 exposure on SARS-CoV-2 genomic RNA levels and N protein expression in lungs (FIGS. 5F and 5G). However, CD4⁺ T cell-depleted and isotype control mice (both OC43 infected) showed indistinguishable features of mild pneumonia in lungs (FIG. 5H), and no difference in SARS-CoV-2 genomic RNA levels in nasal turbinates (FIG. 10B). These data therefore indicate that OC43-elicited CD4⁺ T cells contribute to cross-protection against SARS-CoV-2 infection but do not significantly affect lung disease at day 3 after infection.

These studies with HLA-B*0702 and HLA-DRB1*0101 Ifnar1^{-/-} mice demonstrate that prior exposure to OC43 elicits CD8⁺ and CD4⁺ T cells with cross-reactivity against human-relevant SARS-CoV-2 epitopes, and that these cells contribute to protection against subsequent SARS-CoV-2 infection.

The emergence of novel SARS-CoV-2 variants has highlighted the relatively narrow immune response elicited by the currently available vaccines, and breakthrough infections and reinfections are becoming increasingly prevalent. Moreover, a substantial fraction of these breakthrough infections has occurred in immunocompromised people, and viral evolution in such settings may lead to the generation of new VOCs¹⁰¹, In contrast to the anti-SARS-CoV-2 antibody response, which is focused on the highly variable S protein, T cells contribute to protection against SARS-CoV-2 by recognizing conserved epitopes from multiple SARS-CoV-2 proteins^{102, 103, 104, 105, 106}, particularly in the setting of impaired humoral immunity^{107, 108, 109, 110, 111, 112}. T cells that recognize homologous epitopes from seasonal HCoVs are also present in healthy individuals previously unexposed to SARS-CoV-2^{18, 21, 23, 29, 79, 80, 113, 114}, and robust cross-reactive T cell responses that are rapidly induced following SARS-CoV-2 exposure have been associated with less severe COVID-19, suggesting a role for these cells in protective immunity to SARS-CoV-2^{23, 29, 38, 78, 115}. A greater understanding of the pre-existing SARS-CoV-2 cross-reactive T cell repertoire and response to infection was therefore critical for the development of pan-CoV vaccines that could provide broad protection against current and future SARS-CoV-2 variants and related HCoVs. Pre-existing cross-reactive immune responses contribute to either protection or pathogenesis infection with related viruses⁵⁷, which help to explain the broad heterogeneity in COVID-19 outcomes.

The inventors developed a model of SARS-CoV-2 infection in HLA-B*0702 and HLA-DRB1*0101 transgenic Ifnar1^{-/-} mice with a single pre-exposure to OC43. It was found that primary SARS-CoV-2 infection or vaccination with SARS-CoV-2 protein-encoding vectors in naïve HLA-B*0702 and HLA-DRB1*0101 transgenic Ifnar1^{-/-} mice elicited CD8⁺ and CD4⁺ T cell responses that recapitulated the epitope specificity, Tc1- and Th1-bias, and monofunctional/multifunctional phenotypes observed in SARS-CoV-2-infected and vaccinated humans^{18, 21, 23, 29, 79, 90, 113, 188}. Sequential infection of these animals with OC43 followed by SARS-CoV-2 showed that OC43 pre-exposure protected against SARS-CoV-2 infection in a manner partly dependent on OC43-elicited CD8⁺ and CD4⁺ T cells.

The inventors found that CD8⁺ T cells specific for or cross-reactive with SARS-CoV-2 N_{104/105-113} epitope were immunodominant. Importantly, the inventors observed that immunization of naïve HLA-B*0702 transgenic Ifnar1^{-/-} mice with the single SARS-CoV-2 N₁₀₄₋₁₁₃ peptide evoked a response that limited SARS-CoV-2-induced pathogenesis in the lung. Thus, the presence of immunodominant T cells within pre-existing SARS-CoV-2 cross-reactive T cell immunity could help to explain the disparate outcomes of COVID-19 patients.

In summary, these studies demonstrate a protective role for pre-existing HCoV-elicited cross-reactive CD4⁺ and CD8⁺ T cell responses during SARS-CoV-2 infection. Vaccines that are effective in individuals with antibody or B cell deficiencies are urgently needed not only to protect this highly vulnerable population from SARS-CoV-2 infection but also to limit the emergence of new VOCs.

Additionally, vaccines that elicit antibody and T cell responses that cross-protect against SARS-CoV-2 and other HCoVs may afford protection against other coronaviruses with pandemic potential. Finally, because both HLA haplotype and the T cell antigen repertoire may influence the T cell response to SARS-CoV-2, the HLA transgenic mice employed here help to increase the understanding of the factors that dictate the heterogeneity of COVID-19 outcomes, ranging from asymptomatic or mild infections to severe COVID-19 or death¹³².

Study design. In the past 2 years, numerous human cohort studies have revealed that SARS-CoV-2-unexposed individuals harbor CD8⁺ and CD4⁺ T cells that recognize peptides present in both SARS-CoV-2 and human common cold coronavirus (HCoV). These pre-existing cross-reactive T cells have been associated with both protective and pathologic immunity during SARS-CoV-2 infection, suggesting that they may play an important role in dictating the outcomes of infections, which can range from asymptomatic or mild symptoms to severe COVID-19 and death. This study disectes the origin of pre-existing T cells that cross-react with SARS-CoV-2 and their precise roles during SARS-CoV-2 infection in terms of viral load and disease outcomes. To address these critical questions, a mouse model was developed in which HLA class I (HLA-B*0702 Ifnar1^{-/-}) or class II (HLA-DRB1*0101 Ifnar1^{-/-}) transgenic mice are infected sequentially with one of the 4 major seasonal human coronaviruses followed by SARS-CoV-2.

All experiments were performed in strict accordance with recommendations set forth in the National Institutes of Health Guide for the Care and Use of Laboratory Animals and approved by the Institutional Animal Care and Use Committee at the La Jolla Institute for Immunology ABSL2 and ABSL3 (protocol number AP00001242). HLA-DRB1*0101 and HLA-B*0702Ifnar1^{-/-} transgenic mice were bred under pathogen-free conditions at La Jolla Institute for Immunology. The sex ratio for all experiments was approximately 1:1, and all experiments were started when mice were 5 to 7 weeks of age. For tissue collection, mice were euthanized by CO₂ inhalation. Blood samples were collected into serum collection tubes (Sarstedt) from a facial vein/cardiac puncture in ABSL2 or terminal eye bleeding in ABSL3.

Regarding inclusion and exclusion criteria, all samples were included in the analyses unless technical issues were evident such as high cell mortality after cell isolation (<50%). All SARS-CoV-2 infections were performed in a high containment facility and OC43 infections were performed in a biosafety level 2 infectious facility, control groups of non-infected mice were always added. Each experiment included between 3 to 6 mice and each graph represented pooled data from at least 2 independent experiments. All histology experiments were performed blinded by a board-certified veterinary pathologist.

Vaccination and infection. Mice were vaccinated IM (quadriceps) via electroporation with a minimally invasive device¹³⁴ (BTX Agile Pulse system [47-0500N] with a 4×4×5 mm needle array [47-0045]) with 25 μg of S, M or N DNA vaccine and boosted 14 days later in the same manner. Mice were infected IN with 10⁹ GE of OC43 (ATCC, VR-1558), 10⁴ PFU of SARS-CoV-2 MA10 (Leist et al., 2020), or 10⁵ PFU of SARS-CoV-2 B.1.351 (isolate HCoV-19/South Africa/KRISP-K005325/2020, NR-54009). MA10 and B.1.351 were obtained through BEI Resources (NIAID, NIH). SARS-CoV-2 N₁₀₄₋₁₁₃ peptide (250 μg) was diluted in PBS and homogenized in complete Freund's adjuvant (CFA) and the injection site was gently massaged to facilitate dispersion. Three weeks later, the mice were boosted with the same quantity of peptide in incomplete Freund's adjuvant (IFA). For the mock-vaccinated mice, peptide was replaced with DMSO.

DNA vaccine constructs and detection of viral proteins. Plasmids encoding SARS-CoV-2 S, M, or N proteins (SARS-CoV-2/human/USA/WA-CDC-WA1/2020 isolate, GenBank MN985325.1) were synthesized using human codon optimization. Optimized DNA sequences were synthesized (GenScript), digested with KpnI and NotI, and cloned into pVAX1 under the control of human cytomegalovirus immediate-early promoter with a bovine growth hormone polyadenylation signal and kanamycin as a resistance marker. To increase efficiency of translational initiation, Kozak and IgE leader sequences were introduced. Empty pVAX1 vector served as a negative control.

For transfection, 293T cells were seeded at 2×10⁵ cells/well in 24-well plates in DMEM supplemented with 10% fetal bovine serum (FBS), 1% penicillin-streptomycin, and 1% HEPES buffer, and grown to 70%-80% confluence at 37° C. in a 5% CO₂ atmosphere. One hour before transfection, the supernatant was removed and 200 μl of Opti-MEM™ (Thermo Fisher Scientific) was added to each well. Cell monolayers were transfected with DNA vaccines or empty pVAX1 mixed with Lipofectamine® 2000 (1.5 μg/3 μL) per the manufacturer's instructions. After 20 h, cell monolayers were rinsed three times with PBS and protein expression was assessed by immunofluorescence microscopy or flow cytometry.

For microscopy, monolayers were fixed with Cytofix (BD Biosciences) per the manufacturer's instructions, permeabilized with 0.1% Triton X-100 (for S and N proteins) or 0.05% saponin (for M protein) in PBS (10 min at room temperature), blocked with 3% BSA in PBS (30 min at room temperature), and immunolabeled (1 h at room temperature) with mouse monoclonal antibodies against S protein (Thermo Fisher Scientific, MA5-35946, RRID AB2866558) or N protein (Absolute Antibody, Ab01691-3) or a polyclonal antibody against M protein (Prosci, 9157) diluted 1:500 in 0.1% Triton X-100/PBS. Monolayers were then washed three times with PBS, incubated for 1 h at room temperature with Alexa Fluor 488-conjugated goat anti-mouse IgG (Thermo Fisher Scientific, A11001) diluted 1:200 in 3% BSA/PBS, washed three times with PBS, and overlaid with a drop of ProLong™ Gold Antifade Mountant (Thermo Fisher Scientific). Images were captured with a Keyence BZ-X810 fluorescence microscope using with a Plan Fluor 20X/0.5 dry objective.

For flow cytometry, the cell monolayers were trypsinized, fixed and permeabilized with Cytofix/Cytoperm (BD Biosciences), and then incubated with the anti-N protein and secondary antibodies described above, each for 30 min at 4° C. The cells were washed twice with Cytoperm containing 0.1% BSA and once with FACS buffer and were then resuspended in FACS buffer. Data were collected on an LSR II flow cytometer (BD Biosciences) and analyzed using FlowJo software.

Virus propagation and titration. OC43 was propagated for 9 days in HCT-8 cells cultured in complete RPMI (RPMI medium supplemented with 10% FBS, 1% penicillin-streptomycin, and 1% HEPES buffer). The supernatant was collected and virus was concentrated using a gradient-free method with an Amicon Ultra-15 centrifugal filter unit (Millipore Sigma, UFC9100). Each virus batch was titrated by amplifying the M protein gene using genomic RT-qPCR and the following primers: Rev, 5′-AAT GTA AAG ATG GCC GCG TAT T-3′; Fwd, 5′-ATG TTA ACC TT TAA TTG AGG ACT AT-3′ (IDT Integrated DNA Technologies) as described previously¹³⁵. Cycling conditions were as follows: transcription initiation at 48° C. for 30 min, PCR activation at 95° C. for 10 min, and 45 cycles of amplification at 95° C. for 15 s and 60° C. for 1 min. Viral RNA concentration was calculated using a standard curve composed of at least 4 100-fold serial dilutions of in vitro-transcribed OC43 RNA.

SARS-CoV-2 MA10 and B.1.351 were propagated for 3 days in Vero cells (ATCC, CCL81) cultured in Dulbecco's Modified Eagle's Medium (Corning) supplemented with 10% FBS, 1% penicillin-streptomycin, 1% HEPES buffer, and 1% non-essential amino acids. The supernatant was harvested and titrated using a plaque assay¹³⁶. Briefly, 10-fold serially diluted viral supernatants were added to confluent Vero E₆ cells in 24-well plates (8×10⁴ cells/well) for 2 h at 37° C. The supernatants were removed, 1% carboxymethylcellulose medium was added, and the plates were incubated for 3 days. The cells were then fixed with 10% formaldehyde—for 1 h at room temperature and stained with 0.1% crystal violet for 20 min at room temperature. Viral stocks were deep-sequenced by the La Jolla Institute for Immunology Sequencing Core.

Quantification of viral RNA in tissues. Organs were harvested and placed in 1 mL RNA/DNA shield (ZYMO Research, R1100-250) to maintain high-quality RNA and to inactivate the virus. The tissues were then transferred into RLT lysis buffer containing 1% 2-mercaptoethanol and homogenized at 30 Hz for 3 min using a Tissue Lyser II (QIAGEN). Total RNA was extracted using a RNeasy Mini Kit (QIAGEN) and stored at −80° C. SARS-CoV-2 genomic E RNA and subgenomic 7a RNA were quantified by RT-qPCR using the qScript One-Step qRT-PCR Kit (Quanta BioSciences). For the E gene, the following published primer sets¹³⁷ were used: Fwd, 5′-ACA GGT ACG TTA ATA GTT AAT AGC GT-3′; Rev, 5′-ATA TTG CAG CAG TAC GCA CAC A-3′, and Probe, FAM-ACA CTA GCC ATC CTT ACT GCG CTT CG-BBQ. For the 7a gene, modified primer sets¹³⁸ were used: Fwd, 5′-TCC CAG GTA ACA AAC CAA CCA ACT-3′; Rev, 5′-AAA TGG TGA ATT GCC CTC GT-3-′, and Probe, FAM-CAG TAC TTT TAA AAG ACC TT GCT CTT CTG GAA C-Tamra-Q. Viral RNA concentration was calculated using a standard curve composed of 4 100-fold serial dilutions of in vitro-transcribed SARS-CoV-2 RNA (from isolate USA-WA1/2020, ATCC NR-52347).

Production of recombinant SARS-CoV-2 S and N proteins. For SARS-CoV-2 S protein, HEK-293F cells were cultured to approximately 3×10⁶ cells/mL, transfected with 3 μg/mL of Hexapro-Spike DNA mixed with 9 μg/mL of PEI-MAX (Polysciences), and shaken for 4-5 days at 37° C. in an 80% humidity, 5% CO₂ atmosphere. When the cell viability had decreased to 80%, the supernatant was harvested, centrifuged at 6000×g for 20 min to remove residual cells, and the supernatant was mixed with Biolock reagent (IBA Lifesciences, 2-0205-050; 1:300 v/v), stirred for 15 min to overnight at 4° C., and centrifuged again at 6000×g for 30 min to remove the Biolock-conjugated biotin. S protein was purified from the clarified supernatant by affinity chromatography using a Strep-Tactin column (IBA Lifesciences) on an AKTA purifier (GE Healthcare). The protein fractions were pooled and concentration was estimated by UV absorbance at 280 nm. The tags were removed by addition of HRV-3C protease (10% w/w) and the digested protein was further purified by size-exclusion chromatography using tandem Superose S6 Increase columns (GE Healthcare), concentrated using Vivaspin 500-10K filters (Sartorius), aliquoted, flash-frozen using liquid nitrogen, and stored at −80° C.

Codon-optimized human SARS-CoV-2 N was cloned into pET46 vector (Novagen) with an upstream hexahistidine tag followed by an enterokinase and tobacco etch virus (TEV) cleavage site. Plasmid (100 ng) was transformed by heat shock in Rosetta2 pLysS E. coli (Novagen), and starter cultures were grown with 20 g/mL chloramphenicol and 100 g/mL ampicillin in 50 mL Luria-Bertani broth (LB) overnight at 37° C. After 14-16 hours, the starter cultures were used to inoculate 1 L LB cultures containing 100 g/mL ampicillin. When the optical density at 600 nm (OD₆₀₀) reached ˜0.6, protein production was induced by addition of 0.5 mM isopropyl-D-thiogalactopyranoside followed by incubation for 16-18 h at 25° C. The cells were then pelleted, resuspended in binding buffer (50 mM Tris-HCl, pH 8.0, 300 mM NaCl, and 30 mM imidazole) supplemented with 500 U of benzonase (Biotool, B16012) and protease inhibitors (AEBSF, E64, pepstatin A), and lysed using a Microfluidics M-110P microfluidizer. Cellular debris was removed by centrifugation at 25,000×g for 25 min and the supernatant was filtered (0.22 gm pore size). His-coupled SARS-CoV-2 N protein was incubated with nickel-nitrilotriacetic acid (Ni-NTA) beads for 1 h and then eluted in binding buffer containing TEV protease (1 mg/mL, 0.5% wt/wt) to cleave the His-tag. The resulting sample was dialyzed overnight in snakeskin dialysis tubing (3500 kDa pore size) in 50 mM Tris-Cl, pH 8.5, and 300 mM NaCl. The protein was further purified by size-exclusion chromatography using tandem Superose S6 Increase columns, concentrated using Vivaspin 500-10K filters (Sartorius), aliquoted, flash-frozen using liquid nitrogen, and stored at −80° C.

N protein- and S protein-specific IgG ELISAs. High-binding affinity 96-well plates (Costar) were coated overnight with 1 μg/mL of recombinant SARS-CoV-2 S or N protein (as described above) or OC43 S protein (Sino Biological, 40607-V08B), and then blocked with 5% blotting-grade casein (Bio-Rad). All of the following steps were performed at room temperature. Mouse serum samples were diluted 3-fold from 1:30 to 1:810 (S protein) or 1:30 to 1:65, 610 (N protein) in 1% BSA/PBS and added to the coated wells for 1.5 h. The plates were washed 3 times with PBST (PBS with 0.05% Tween-20, pH 7.4), incubated with a 1:5000 dilution of horseradish peroxidase (HRP)-conjugated anti-mouse IgG monoclonal antibody IgG polyclonal antibody (Jackson ImmunoResearch in 1% BSA/PBS, and washed again. Color development was initiated by addition of TMB substrate (Pierce) and the plates were then incubated in the dark for 15 min. The reaction was stopped by addition of 2 N sulfuric acid (Fisher Chemical). OD₄₅₀ was read immediately using a SpectraMax M2 microplate reader (Molecular Devices). The OD cutoff for positive reactivity was 2 standard deviations above the mean OD of the negative control wells (wells coated with antigen but lacking serum).

Flow cytometry and intracellular cytokine staining (ICS) assay. Spleens and lungs were processed to give single-cell suspensions of splenocytes and lung leukocytes, respectively. Briefly, lungs were cut into small pieces, digested with 1 mg/mL type I collagenase (Worthington) and 20 U/mL DNase I (Thermo Fisher Scientific) for 30 min at 37° C., and then mechanically dissociated using a gentleMACS Octo Dissociator. The cell suspension was filtered through a 70-μm cell strainer and red blood cells were lysed with ACK lysing buffer (Gibco). Spleens were gently mashed with a syringe plunger, filtered through a 70-μm cell strainer, and treated with ACK lysing buffer. Splenocytes or lung leukocytes were placed in 96-well round-bottom plates at 2×10⁶ cells/well in complete RPMI and stimulated with 10 μg/mL of SARS-CoV-2 peptides for 1 h at 37° C. Brefeldin A (BioLegend; 1:1000 dilution) and rat anti-mouse CD107a (Clone 1D4B, Biolegend) were then added and the cells were incubated for an additional 4 h. Positive controls were cells stimulated for the same time with Cell Stimulation Cocktail (eBioscience), and negative controls were incubated similarly but without stimulants. At the end of the in vitro incubation, cells were stained with efluor 455 (UV) viability dye (Invitrogen) and fluorophore-conjugated antibodies against mouse CD3E (Tonbo, clone 145-2C11), CD8α (BioLegend, clone 53-6.7), CD44 (BioLegend, clone IM7), and CD62L (BioLegend, MFL-14). Cells were then fixed and permeabilized with Cytofix/Cytoperm and stained with fluorophore-conjugated antibodies against mouse IFNγ (Tonbo, clone XMG1.2), TNFα (eBioscience, clone MP6-XT22), IL-2 (BioLegend, clone JES6-5H4), or CD107a (Biolegend, clone 1D4B). Data were collected on an LSR Fortessa flow cytometer (BD Biosciences) and analyzed using FlowJo software.

IFNγ-ELISpot assay. Single-cell suspensions were prepared from spleens and lungs as described above. Splenocytes or lung leukocytes were placed at 10⁵ cells/well in 96-well flat-bottom plates (Immobilon-P; Millipore, MA) pre-coated with anti-mouse IFNγ antibody (clone AN18; Mabtech, Sweden) and incubated for 20 h at 37° C. with 10 μg/mL of the appropriate SARS-CoV-2 peptides. Plates were processed as previously described¹³⁹, and spot-forming cells (SFCs) were counted using an ELISpot reader (MABTech).

CD4⁺ and CD8⁺ T cell depletion. Mice were injected with 250 μg of CD8⁺ T cell-depleting antibody (BioXCell, clone 2.43 CD4⁺ T cell-depleting antibody (BioXCell, clone GK1.5), or rat IgG2 isotype control antibody (BioXCell, clone LTF-2) intraperitoneally on days −3, −2, and −1 before SARS-CoV-2 challenge. Blood was collected prior to SARS-CoV-2 challenge and analyzed by flow cytometry to validate CD8⁺ or CD4⁺ T cell depletion.

Peptide prediction, selection, and immunization. HLA-DRB1*0101-restricted and HLA-B*0702-restricted SARS-CoV2 N, S, and M-protein-derived T cell epitopes were identified as follows. Protein sequences for SARS-CoV-2/human/USA/WA-CDC-WA1/2020 isolate (GenBank MN985325.1) were accessed via the NCBI protein database. Using the Immune Epitope DataBase (www.iedb.org) website tools and the “IEDB-recommended” method selection, MHC class II or class I peptide binding affinity predictions were obtained for all non-redundant 15-mer peptides that bind to the HLA-DRB1*0101 allele or for all 8- to 11-mer peptides that bind to the HLA-B*0702 allele. The resulting peptide lists were sorted by increasing consensus percentile rank, and the top 1% were selected (Tables 2 and 3). Selected peptides were synthesized and purified to >95% purity by TC Peptide Lab (San Diego) by reverse-phase HPLC, and validated by mass spectrometry. Peptides were dissolved in DMSO for use.

HLA-DRB1*0101 and HLA-B*0702-restricted SARS-CoV2 proteome-derived T cell epitopes were searched on the NIAID Virus Pathogen Database and Analysis Resource (https://www.viprbrc.org/; accessed May 2, 2021) by querying the virus species name “severe acute respiratory syndrome-related coronavirus” from “human” hosts. The inventors limited the search to epitopes identified by at least one of the following T cell assays: ELISpot, ICS, or MHC-binding assays. The resulting 37 predicted HLA-DRB1*0101 and HLA-B*0702 epitopes (Table 4 and 5) were synthesized as crude material (1 mg scale) by TC Peptide Lab (San Diego).

TABLE 5
HLA-DRB1*0101-restricted CD4+ T cell epitopes
identified

| | | | | | SEQ ID |
|---|---|---|---|---|---|
| Protein | Start | End | Length | Sequence | NO: |
| Spike | 530 | 544 | 15 | STDLIKNQCVNFNFN | 149 |
| Envelope | 26 | 40 | 15 | FLLVTLAILTALRLC | 150 |
| Membrane | 36 | 50 | 15 | QFAYANRNRFLYIIK | 151 |
| Membrane | 66 | 80 | 15 | VLAAVYRINWITGGI | 152 |
| Membrane | 71 | 85 | 15 | YRINWITGGIAIAMA | 153 |
| Membrane | 86 | 100 | 15 | CLVGLMWLSYFIASF | 154 |
| Membrane | 91 | 105 | 15 | MWLSYFIASFRLFAR | 155 |
| Membrane | 116 | 130 | 15 | TNILLNVPLHGTILT | 156 |
| Membrane | 136 | 150 | 15 | SELVIGAVILRGHLR | 157 |
| Membrane | 146 | 160 | 15 | RGHLRIAGHHLGRCD | 158 |
| Membrane | 151 | 165 | 15 | IAGHHLGRCDIKDLP | 159 |
| Membrane | 161 | 175 | 15 | IKDLPKEITVATSRT | 160 |
| Membrane | 166 | 180 | 15 | KEITVATSRTLSYYK | 161 |
| Membrane | 176 | 190 | 15 | LSYYKLGASQRVAGD | 162 |
| Membrane | 191 | 205 | 15 | SGFAAYSRYRIGNYK | 163 |
| Nucleo- | 81 | 95 | 15 | DDQIGYYRRATRRIR | 164 |
| protein | | | | | |
| Nucleo- | 86 | 100 | 15 | YYRRATRRIRGGDGK | 165 |
| protein | | | | | |
| Nucleo- | 126 | 140 | 15 | NKDGIIWVATEGALN | 166 |
| protein | | | | | |
| Nucleo- | 211 | 225 | 15 | AGNGGDAALALLLLD | 167 |
| protein | | | | | |
| Nucleo- | 216 | 230 | 15 | DAALALLLLDRLNQL | 168 |
| protein | | | | | |
| Nucleo- | 221 | 235 | 15 | LLLLDRLNQLESKMS | 169 |
| protein | | | | | |
| Nucleo- | 261 | 275 | 15 | KRTATKAYNVTQAFG | 170 |
| protein | | | | | |
| Nucleo- | 301 | 315 | 15 | WPQIAQFAPSASAFF | 171 |
| protein | | | | | |
| Nucleo- | 317 | 331 | 15 | MSRIGMEVTPSGTWL | 172 |
| protein | | | | | |
| Nucleo- | 326 | 340 | 15 | PSGTWLTYTGAIKLD | 173 |
| protein | | | | | |
| Nucleo- | 346 | 360 | 15 | FKDQVILLNKHIDAY | 174 |
| protein | | | | | |
| Nucleo- | 351 | 365 | 15 | ILLNKHIDAYKTFPP | 175 |
| protein | | | | | |
| ORF1ab | 5041 | 5055 | 15 | SHRFYRLANECAQVL | 176 |
| ORF1ab | 5246 | 5260 | 15 | LMIERFVSLAIDAYP | 177 |
| ORF3a | 106 | 120 | 15 | LYLYALVYFLQSINF | 178 |
| ORF3a | 116 | 130 | 15 | QSINFVRIIMRLWLC | 179 |
| ORF8 | 36 | 50 | 15 | PCPIHFYSKWYIRVG | 180 |
| ORF8 | 41 | 55 | 15 | FYSKWYIRVGARKSA | 181 |
| ORF8 | 76 | 90 | 15 | IGNYTVSCLPFTINC | 182 |
| ORF8 | 86 | 100 | 15 | FTINCQEPKLGSLVV | 183 |
| ORF8 | 96 | 110 | 15 | GSLVVRCSFYEDFLE | 184 |
| ORF8 | 101 | 115 | 15 | RCSFYEDFLEYHDVR | 185 |

Histopathology. Lungs were fixed with zinc formalin for 24 h at room temperature and transferred to 70% alcohol. Turbinates were decalcified using 5% formic acid (Fisher Scientific #A118P-4). Tissues were then embedded in paraffin using standard procedures, sliced into 4-μM sections, stained with H&E using a Leica ST5020 autostainer, and imaged using a Zeiss AxioScan Zi with a 40×0.95 NA objective. Histopathological analysis was performed by a board-certified veterinary pathologist who was blinded to the experimental condition and group identity. Sections were scored (0-5) for 10 criteria for SARS-CoV-2-induced pneumonia, as seen in hamsters, macaques, and COVID-19 patients¹⁰⁰.

Statistical analysis. Data are expressed as the mean±standard error (SEM) and were analyzed with Prism software v9.1.1 (GraphPad Software, La Jolla, CA, USA). Differences between group means were analyzed by the Kruskal-Wallis test, (more than 2 groups), nonparametric Mann-Whitney test (2 groups), and 2-way ANOVA followed by Sidak's multiple comparisons test (more than 2 groups, more than one variables). P<0.05 was considered statistically significant.

Other examples of implementations will become apparent to the reader in view of the teachings of the present description and as such, will not be further described here.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

Note that titles or subtitles may be used throughout the present disclosure for convenience of a reader, but in no way these should limit the scope of the invention. Moreover, certain theories may be proposed and disclosed herein; however, in no way they, whether they are right or wrong, should limit the scope of the invention so long as the invention is practiced according to the present disclosure without regard for any particular theory or scheme of action.

All references cited throughout the specification are hereby incorporated by reference in their entirety for all purposes.

It will be understood by those of skill in the art that throughout the present specification, the term “a” used before a term encompasses embodiments containing one or more to what the term refers. It will also be understood by those of skill in the art that throughout the present specification, the term “comprising”, which is synonymous with “including,” “containing,” or “characterized by,” is inclusive or open-ended and does not exclude additional, un-recited elements or method steps.

Unless otherwise defined, all technical and scientific terms used herein have the same meaning as commonly understood by one of ordinary skill in the art to which this invention pertains. In the case of conflict, the present document, including definitions will control.

As used in the present disclosure, the terms “around”, “about” or “approximately” shall generally mean within the error margin generally accepted in the art. Hence, numerical quantities given herein generally include such error margin such that the terms “around”, “about” or “approximately” can be inferred if not expressly stated.

Although various embodiments of the disclosure have been described and illustrated, it will be apparent to those skilled in the art in light of the present description that numerous modifications and variations can be made. The scope of the invention is defined more particularly in the appended claims.

### REFERENCES

1. Noh J Y, Jeong H W, Shin E C. SARS-CoV-2 mutations, vaccines, and immunity: implication of variants of concern. Signal Transduct Target Ther 6, 203 (2021).
2. Andrews N, et al. Covid-19 Vaccine Effectiveness against the Omicron (B.1.1.529) Variant. The New England journal of medicine 386, 1532-1546 (2022).
3. Geers D, et al. SARS-CoV-2 variants of concern partially escape humoral but not T-cell responses in COVID-19 convalescent donors and vaccinees. Science immunology 6, (2021).
4. Nasreen S, et al. Effectiveness of COVID-19 vaccines against symptomatic SARS-CoV-2 infection and severe outcomes with variants of concern in Ontario. Nat Microbiol 7, 379-385 (2022).
5. Wratil P R, et al. Three exposures to the spike protein of SARS-CoV-2 by either infection or vaccination elicit superior neutralizing immunity to all variants of concern. Nat Med 28, 496-503 (2022).
6. Sakurai A, et al. Natural History of Asymptomatic SARS-CoV-2 Infection. The New England journal of medicine 383, 885-886 (2020).
7. Spudich S, Nath A. Nervous system consequences of COVID-19. Science (New York, N Y) 375, 267-269 (2022).
8. Xie Y, Bowe B, Al-Aly Z. Burdens of post-acute sequelae of COVID-19 by severity of acute infection, demographics and health status. Nat Commun 12, 6571 (2021).
9. Xie Y, Xu E, Bowe B, Al-Aly Z. Long-term cardiovascular outcomes of COVID-19. Nat Med 28, 583-590 (2022).
10. Guan W J, et al. Clinical Characteristics of Coronavirus Disease 2019 in China. The New England journal of medicine 382, 1708-1720 (2020).
11. Al-Aly Z, Xie Y, Bowe B. High-dimensional characterization of post-acute sequelae of COVID-19. Nature 594, 259-264 (2021).
12. Muscogiuri G, et al. Low-grade inflammation, CoVID-19, and obesity: clinical aspect and molecular insights in childhood and adulthood. Int J Obes (Lond), (2022).
13. Ng W H, et al. Comorbidities in SARS-CoV-2 Patients: a Systematic Review and Meta-Analysis. mBio 12, (2021).
14. Price-Haywood E G, Burton J, Fort D, Seoane L. Hospitalization and Mortality among Black Patients and White Patients with Covid-19. The New England journal of medicine 382, 2534-2543 (2020).
15. O'Driscoll M, et al. Age-specific mortality and immunity patterns of SARS-CoV-2. Nature 590, 140-145 (2021).
16. Klang E, Kassim G, Soffer S, Freeman R, Levin M A, Reich D L. Severe Obesity as an Independent Risk Factor for COVID-19 Mortality in Hospitalized Patients Younger than 50. Obesity (Silver Spring) 28, 1595-1599 (2020).
17. Jin J M, et al. Gender Differences in Patients With COVID-19: Focus on Severity and Mortality. Front Public Health 8, 152 (2020).
18. Mateus J, et al. Selective and cross-reactive SARS-CoV-2 T cell epitopes in unexposed humans. Science (New York, N Y) 370, 89-94 (2020).
19. Dykema A G, et al. Functional characterization of CD4+ T cell receptors crossreactive for SARS-CoV-2 and endemic coronaviruses. J Clin Invest 131, (2021).
20. Woldemeskel B A, Dykema A G, Garliss C C, Cherfils S, Smith K N, Blankson J N. CD4+ T cells from COVID-19 mRNA vaccine recipients recognize a conserved epitope present in diverse coronaviruses. J Clin Invest 132, (2022).
21. Le Bert N, et al. SARS-CoV-2-specific T cell immunity in cases of COVID-19 and SARS, and uninfected controls. Nature 584, 457-462 (2020).
22. Tan H X, et al. Adaptive immunity to human coronaviruses is widespread but low in magnitude. Clin Transl Immunology 10, e1264 (2021).
23. Grifoni A, et al. Targets of T Cell Responses to SARS-CoV-2 Coronavirus in Humans with COVID-19 Disease and Unexposed Individuals. Cell 181, 1489-1501.e1415 (2020).
24. Ni L, et al. Detection of SARS-CoV-2-Specific Humoral and Cellular Immunity in COVID-19 Convalescent Individuals. Immunity 52, 971-977 e973 (2020).
25. Peng Y, et al. Broad and strong memory CD4(+) and CD8(+) T cells induced by SARS-CoV-2 in U K convalescent individuals following COVID-19. Nat Immunol 21, 1336-1345 (2020).
26. Weiskopf D, et al. Phenotype and kinetics of SARS-CoV-2-specific T cells in COVID-19 patients with acute respiratory distress syndrome. Science immunology 5, (2020).
27. Kundu R, et al. Cross-reactive memory T cells associate with protection against SARS-CoV-2 infection in COVID-19 contacts. Nat Commun 13, 80 (2022).
28. Lineburg K E, et al. CD8(+) T cells specific for an immunodominant SARS-CoV-2 nucleocapsid epitope cross-react with selective seasonal coronaviruses. Immunity 54, 1055-1065 e1055 (2021).
29. Braun J, et al. SARS-CoV-2-reactive T cells in healthy donors and patients with COVID-19. Nature 587, 270-274 (2020).
30. Garcia-Jimenez A F, et al. Cross-reactive cellular, but not humoral, immunity is detected between OC43 and SARS-CoV-2 NPs in people not infected with SARS-CoV-2: Possible role of cTFH cells. J Leukoc Biol, (2022).
31. Mesel-Lemoine M, et al. A human coronavirus responsible for the common cold massively kills dendritic cells but not monocytes. J Virol 86, 7577-7587 (2012).
32. Gorse G J, Patel G B, Vitale J N, O'Connor T Z. Prevalence of antibodies to four human coronaviruses is lower in nasal secretions than in serum. Clin Vaccine Immunol 17, 1875-1880 (2010).
33. Ellis P, Somogyvari F, Virok D P, Noseda M, McLean G R. Decoding Covid-19 with the SARS-CoV-2 Genome. Curr Genet Med Rep 9, 1-12 (2021).
34. Loyal L, et al. Cross-reactive CD4(+) T cells enhance SARS-CoV-2 immune responses upon infection and vaccination. Science (New York, N Y) 374, eabh1823 (2021).
35. Mateus J, et al. Low-dose mRNA-1273 COVID-19 vaccine generates durable memory enhanced by cross-reactive T cells. Science (New York, N Y) 374, eabj9853 (2021).
36. Bacher P, et al. Low-Avidity CD4(+) T Cell Responses to SARS-CoV-2 in Unexposed Individuals and Humans with Severe COVID-19. Immunity 53, 1258-1271 e1255 (2020).
37. Gouma S, et al. Health care worker seromonitoring reveals complex relationships between common coronavirus antibodies and COVID-19 symptom duration. JCI Insight 6, (2021).
38. Mallajosyula V, et al. CD8(+) T cells specific for conserved coronavirus epitopes correlate with milder disease in COVID-19 patients. Science immunology 6, (2021).
39. Bonifacius A, et al. COVID-19 immune signatures reveal stable antiviral T cell function despite declining humoral responses. Immunity 54, 340-354 e346 (2021).
40. Zellweger R M, Prestwood T R, Shresta S. Enhanced infection of liver sinusoidal endothelial cells in a mouse model of antibody-induced severe dengue disease. Cell Host Microbe 7, 128-139 (2010).
41. Zellweger R M, Tang W W, Eddy W E, King K, Sanchez M C, Shresta S. CD8+ T Cells Can Mediate Short-Tenn Protection against Heterotypic Dengue Virus Reinfection in Mice. J Virol 89, 6494-6505 (2015).
42. Balsitis S J, et al. Lethal antibody enhancement of dengue disease in mice is prevented by Fc modification. PLoS Pathog 6, e1000790 (2010).
43. Wen J, et al. Dengue virus-reactive CD8(+) T cells mediate cross-protection against subsequent Zika virus challenge. Nat Commun 8, 1459 (2017).
44. Wen J, et al. CD4(+) T Cells Cross-Reactive with Dengue and Zika Viruses Protect against Zika Virus Infection. Cell Rep 31, 107566 (2020).
45. Regla-Nava J A, et al. Cross-reactive Dengue virus-specific CD8(+) T cells protect against Zika virus during pregnancy. Nat Commun 9, 3042 (2018).
46. Katzelnick L C, et al. Zika virus infection enhances future risk of severe dengue disease. Science (New York, N Y) 369, 1123-1128 (2020).
47. Katzelnick L C, et al. Antibody-dependent enhancement of severe dengue disease in humans. Science (New York, N Y) 358, 929-932 (2017).
48. Salje H, et al. Reconstruction of antibody dynamics and infection histories to evaluate dengue risk. Nature 557, 719-723 (2018).
49. Fowler A M, et al. Maternally Acquired Zika Antibodies Enhance Dengue Disease Severity in Mice. Cell Host Microbe 24, 743-750 e745 (2018).
50. Gordon A, et al. Prior dengue virus infection and risk of Zika: A pediatric cohort in Nicaragua. PLoS Med 16, e1002726 (2019).
51. Rodriguez-Barraquer I, et al. Impact of preexisting dengue immunity on Zika virus emergence in a dengue endemic region. Science (New York, N Y) 363, 607-610 (2019).
52. Pedroso C, et al. Cross-Protection of Dengue Virus Infection against Congenital Zika Syndrome, Northeastern Brazil. Emerg Infect Dis 25, 1485-1493 (2019).
53. Sridhar S, et al. Effect of Dengue Serostatus on Dengue Vaccine Safety and Efficacy. The New England journal of medicine 379, 327-340 (2018).
54. Sharp™, et al. Knowledge gaps in the epidemiology of severe dengue impede vaccine evaluation. Lancet Infect Dis 22, e42-e51 (2022).
55. Katzelnick L C, Bos S, Harris E. Protective and enhancing interactions among dengue viruses 1-4 and Zika virus. Curr Opin Virol 43, 59-70 (2020).
56. Valentine K M, Croft M, Shresta S. Protection against dengue virus requires a sustained balance of antibody and T cell responses. Curr Opin Virol 43, 22-27 (2020).
57. Ngono A E, Shresta S. Immune Response to Dengue and Zika. Annu Rev Immunol 36, 279-308 (2018).
58. Hassert M, Brien J D, Pinto A K. Mouse Models of Heterologous Flavivirus Immunity: A Role for Cross-Reactive T Cells. Front Immunol 10, 1045 (2019).
59. Gonzalez-Galarza F F, et al. Allele frequency net database (AFND) 2020 update: gold-standard data classification, open access genotype data and new query tools. Nucleic Acids Res 48, D783-D788 (2020).
60. Solberg O D, et al. Balancing selection and heterogeneity across the classical human leukocyte antigen loci: a meta-analytic review of 497 population studies. Hum Immunol 69, 443-464 (2008).
61. Bastard P, et al. Autoantibodies neutralizing type I IFNs are present in ˜4% of uninfected individuals over 70 years old and account for ˜20% of COVID-19 deaths. Science immunology 6, (2021).
62. Bastard P, et al. Autoantibodies against type I IFNs in patients with life-threatening COVID-19. Science (New York, N Y) 370, (2020).
63. Koning R, Bastard P, Casanova J L, Brouwer M C, van de Beek D, with the Amsterdam UMCC-BI. Autoantibodies against type I interferons are associated with multi-organ failure in COVID-19 patients. Intensive Care Med 47, 704-706 (2021).
64. Troya J, et al. Neutralizing Autoantibodies to Type I IFNs in >10% of Patients with Severe COVID-19 Pneumonia Hospitalized in Madrid, Spain. J Clin Immunol 41, 914-922 (2021).
65. Vazquez S E, et al. Neutralizing Autoantibodies to Type I Interferons in COVID-19 Convalescent Donor Plasma. J Clin Immunol 41, 1169-1171 (2021).
66. Wang E Y, et al. Diverse functional autoantibodies in patients with COVID-19. Nature 595, 283-288 (2021).
67. Zhang Q, et al. Inborn errors of type I IFN immunity in patients with life-threatening COVID-19. Science (New York, N Y) 370, (2020).
68. Elong Ngono A, et al. Protective Role of Cross-Reactive CD8 T Cells Against Dengue Virus Infection. EBioMedicine 13, 284-293 (2016).
69. Weiskopf D, Angelo M A, Sidney J, Peters B, Shresta S, Sette A. Immunodominance changes as a function of the infecting dengue virus serotype and primary versus secondary infection. Journal of virology 88, 11383-11394 (2014).
70. Weiskopf D, et al. Insights into HLA-restricted T cell responses in a novel mouse model of dengue virus infection point toward new implications for vaccine design. J Immunol 187, 4268-4279 (2011).
71. Wen J, et al. Identification of Zika virus epitopes reveals immunodominant and protective roles for dengue virus cross-reactive CD8(+) T cells. Nat Microbiol 2, 17036 (2017).
72. Leist S R, et al. A Mouse-Adapted SARS-CoV-2 Induces Acute Lung Injury and Mortality in Standard Laboratory Mice. Cell 183, 1070-1085 e1012 (2020).
73. Vita R, et al. The Immune Epitope Database (IEDB): 2018 update. Nucleic Acids Res 47, D339-D343 (2019).
74. Liu Y, et al. The N501Y spike substitution enhances SARS-CoV-2 infection and transmission. Nature 602, 294-299 (2022).
75. Pan T, et al. Infection of wild-type mice by SARS-CoV-2 B.1.351 variant indicates a possible novel cross-species transmission route. Signal Transduct Target Ther 6, 420 (2021).
76. Ferretti A P, et al. Unbiased Screens Show CD8(+) T Cells of COVID-19 Patients Recognize Shared Epitopes in SARS-CoV-2 that Largely Reside outside the Spike Protein. Immunity 53, 1095-1107 e1093 (2020).
77. Nguyen T H O, et al. CD8(+) T cells specific for an immunodominant SARS-CoV-2 nucleocapsid epitope display high naive precursor frequency and TCR promiscuity. Immunity 54, 1066-1082 e1065 (2021).
78. Schulien I, et al. Characterization of pre-existing and induced SARS-CoV-2-specific CD8(+) T cells. Nat Med 27, 78-85 (2021).
79. Sekine T, et al. Robust T Cell Immunity in Convalescent Individuals with Asymptomatic or Mild COVID-19. Cell 183, 158-168 e114 (2020).
80. Painter M M, et al. Rapid induction of antigen-specific CD4(+) T cells is associated with coordinated humoral and cellular immunity to SARS-CoV-2 mRNA vaccination. Immunity 54, 2133-2142 e2133 (2021).
81. Dan J M, et al. Immunological memory to SARS-CoV-2 assessed for up to 8 months after infection. Science (New York, N Y) 371, (2021).
82. Hicks J, et al. Serologic Cross-Reactivity of SARS-CoV-2 with Endemic and Seasonal Betacoronaviruses. J Clin Immunol 41, 906-913 (2021).
83. Saletti G, et al. Older adults lack SARS CoV-2 cross-reactive T lymphocytes directed to human coronaviruses OC43 and NL63. Sci Rep 10, 21447 (2020).
84. Nickbakhsh S, Ho A, Marques D F P, McMenamin J, Gunson R N, Murcia P R. Epidemiology of Seasonal Coronaviruses: Establishing the Context for the Emergence of Coronavirus Disease 2019. J Infect Dis 222, 17-25 (2020).
85. Killerby M E, et al. Human coronavirus circulation in the United States 2014-2017. Journal of clinical virology: the official publication of the Pan American Society for Clinical Virology 101, 52-56 (2018).
86. Snyder™, et al. Magnitude and Dynamics of the T-Cell Response to SARS-CoV-2 Infection at Both Individual and Population Levels. medRxiv: the preprint server for health sciences, (2020).
87. Tarke A, et al. Comprehensive analysis of T cell immunodominance and immunoprevalence of SARS-CoV-2 epitopes in COVID-19 cases. Cell reports Medicine 2, 100204 (2021).
88. Saini S K, et al. SARS-CoV-2 genome-wide T cell epitope mapping reveals immunodominance and substantial CD8(+) T cell activation in COVID-19 patients. Science immunology 6, (2021).
89. Quadeer A A, Ahmed S F, McKay M R. Landscape of epitopes targeted by T cells in 852 individuals recovered from COVID-19: Meta-analysis, immunoprevalence, and web platform. Cell reports Medicine 2, 100312 (2021).
90. Nelde A, et al. SARS-CoV-2-derived peptides define heterologous and COVID-19-induced T cell recognition. Nat Immunol 22, 74-85 (2021).
91. Grifoni A, Sidney J, Zhang Y, Scheuermann R H, Peters B, Sette A. A Sequence Homology and Bioinformatic Approach Can Predict Candidate Targets for Immune Responses to SARS-CoV-2. Cell Host Microbe 27, 671-680 e672 (2020).
92. Kared H, et al. SARS-CoV-2-specific CD8+ T cell responses in convalescent COVID-19 individuals. J Clin Invest 131, (2021).
93. Ng O W, et al. Memory T cell responses targeting the SARS coronavirus persist up to 11 years post-infection. Vaccine 34, 2008-2014 (2016).
94. Prakash S, et al. Genome-Wide B Cell, CD4(+), and CD8(+) T Cell Epitopes That Are Highly Conserved between Human and Animal Coronaviruses, Identified from SARS-CoV-2 as Targets for Preemptive Pan-Coronavirus Vaccines. J Immunol 206, 2566-2582 (2021).
95. Shen Y, et al. Ancestral origins are associated with SARS-CoV-2 susceptibility and protection in a Florida patient population. bioRxiv, 2022.2003.2030.486345 (2022).
96. Keller M D, et al. SARS-CoV-2-specific T cells are rapidly expanded for therapeutic use and target conserved regions of the membrane protein. Blood 136, 2905-2917 (2020).
97. Tan A T, et al. Early induction of functional SARS-CoV-2-specific T cells associates with rapid viral clearance and mild disease in COVID-19 patients. Cell Rep 34, 108728 (2021).
98. Crotty S. T follicular helper cell differentiation, function, and roles in disease. Immunity 41, 529-542 (2014).
99. Ueno H, Banchereau J, Vinuesa C G. Pathophysiology of T follicular helper cells in humans and mice. Nat Immunol 16, 142-152 (2015).
100. Gruber A D, et al. Standardization of Reporting Criteria for Lung Pathology in SARS-CoV-2-infected Hamsters: What Matters? Am J Respir Cell Mol Biol 63, 856-859 (2020).
101. Corey L, Beyrer C, Cohen M S, Michael N L, Bedford T, Rolland M. SARS-CoV-2 Variants in Patients with Immunosuppression. The New England journal of medicine 385, 562-566 (2021).
102. Redd A D, et al. CD8+ T-Cell Responses in COVID-19 Convalescent Individuals Target Conserved Epitopes From Multiple Prominent SARS-CoV-2 Circulating Variants. Open Forum Infect Dis 8, ofab143 (2021).
103. Naranbhai V, et al. T cell reactivity to the SARS-CoV-2 Omicron variant is preserved in most but not all individuals. Cell 185, 1041-1051 e1046 (2022).
104. Keeton R, et al. T cell responses to SARS-CoV-2 spike cross-recognize Omicron. Nature 603, 488-492 (2022).
105. Gao Y, et al. Ancestral SARS-CoV-2-specific T cells cross-recognize the Omicron variant. Nat Med 28, 472-476 (2022).
106. GeurtsvanKessel C H, et al. Divergent SARS-CoV-2 Omicron-reactive T and B cell responses in COVID-19 vaccine recipients. Science immunology 7, eabo2202 (2022).
107. Sun J, et al. Generation of a Broadly Useful Model for COVID-19 Pathogenesis, Vaccination, and Treatment. Cell 182, 734-743.e735 (2020).
108. McMahan K, et al. Correlates of protection against SARS-CoV-2 in rhesus macaques. Nature 590, 630-634 (2021).
109. Soresina A, et al. Two X-linked agammaglobulinemia patients develop pneumonia as COVID-19 manifestation but recover. Pediatr Allergy Immunol 31, 565-569 (2020).
110. Bange E M, et al. CD8(+) T cells contribute to survival in patients with COVID-19 and hematologic cancer. Nat Med 27, 1280-1289 (2021).
111. Zhuang Z, et al. Mapping and role of T cell response in SARS-CoV-2-infected mice. J Exp Med 218, (2021).
112. Israelow B, et al. Adaptive immune determinants of viral clearance and protection in mouse models of SARS-CoV-2. Science immunology 6, eabl4509 (2021).
113. Rydyznski Moderbacher C, et al. Antigen-Specific Adaptive Immunity to SARS-CoV-2 in Acute COVID-19 and Associations with Age and Disease Severity. Cell 183, 996-1012.e1019 (2020).
114. Nesterenko P A, et al. HLA-A(*)02:01 restricted T cell receptors against the highly conserved SARS-CoV-2 polymerase cross-react with human coronaviruses. Cell Rep 37, 110167 (2021).
115. Le Bert N, et al. Highly functional virus-specific cellular immune response in asymptomatic SARS-CoV-2 infection. J Exp Med 218, (2021).
116. Tan C C S, Owen C J, Tham C Y L, Bertoletti A, van Dorp L, Balloux F. Pre-existing T cell-mediated cross-reactivity to SARS-CoV-2 cannot solely be explained by prior exposure to endemic human coronaviruses. Infect Genet Evol 95, 105075 (2021).
117. Eggenhuizen P J, et al. Heterologous Immunity Between SARS-CoV-2 and Pathogenic Bacteria. Front Immunol 13, 821595 (2022).
118. Low J S, et al. Clonal analysis of immunodominance and cross-reactivity of the CD4 T cell response to SARS-CoV-2. Science (New York, N Y) 372, 1336-1341 (2021).
119. Sagar M, et al. Recent endemic coronavirus infection is associated with less-severe COVID-19. J Clin Invest 131, (2021).
120. Zhao J, et al. Airway Memory CD4(+) T Cells Mediate Protective Immunity against Emerging Respiratory Coronaviruses. Immunity 44, 1379-1391 (2016).
121. Poston D, et al. Absence of Severe Acute Respiratory Syndrome Coronavirus 2 Neutralizing Activity in Prepandemic Sera From Individuals With Recent Seasonal Coronavirus Infection. Clin Infect Dis 73, e1208-e1211 (2021).
122. Ercanoglu M S, et al. No substantial preexisting B cell immunity against SARS-CoV-2 in healthy adults. iScience 25, 103951 (2022).
123. Pinto D, et al. Broad betacoronavirus neutralization by a stem helix-specific human antibody. Science (New York, N Y) 373, 1109-1116 (2021).
124. Sun X, et al. Neutralization mechanism of a human antibody with pan-coronavirus reactivity including SARS-CoV-2. Nat Microbiol 7, 1063-1074 (2022).
125. Dacon C, et al. Broadly neutralizing antibodies target the coronavirus fusion peptide. Science (New York, N Y) 377, 728-735 (2022).
126. Low J S, et al. ACE2-binding exposes the SARS-CoV-2 fusion peptide to broadly neutralizing coronavirus antibodies. Science (New York, N Y) 377, 735-742 (2022).
127. Francis J M, et al. Allelic variation in class I HLA determines CD8(+) T cell repertoire shape and cross-reactive memory responses to SARS-CoV-2. Science immunology 7, eabk3070 (2022).
128. Dangi T, et al. Cross-protective immunity following coronavirus vaccination and coronavirus infection. J Clin Invest 131, (2021).
129. Grifoni A, et al. Prior Dengue Virus Exposure Shapes T Cell Immunity to Zika Virus in Humans. J Virol 91, (2017).
130. Wragg K M, et al. Establishment and recall of SARS-CoV-2 spike epitope-specific CD4(+) T cell memory. Nat Immunol, (2022).
131. Heitmann J S, et al. A COVID-19 peptide vaccine for the induction of SARS-CoV-2 T cell immunity. Nature 601, 617-622 (2022).
132. Minervina A A, et al. SARS-CoV-2 antigen exposure history shapes phenotypes and specificity of memory CD8 T cells. medRxiv: the preprint server for health sciences, (2022).
133. Bastard P. Why do people die from COVID-19? Science (New York, N Y) 375, 829-830 (2022).
134. Choi H, et al. Protective immunity by an engineered DNA vaccine for Mayaro virus. PLoS Negl Trop Dis 13, e0007042 (2019).
135.Vijgen L, Keyaerts E, Moes E, Maes P, Duson G, Van Ranst M. Development of one-step, real-time, quantitative reverse transcriptase PCR assays for absolute quantitation of human coronaviruses OC43 and 229E. J Clin Microbiol 43, 5452-5456 (2005).
136. Mendoza E J, Manguiat K, Wood H, Drebot M. Two Detailed Plaque Assay Protocols for the Quantification of Infectious SARS-CoV-2. Curr Protoc Microbiol 57, ecpmc105 (2020).
137. Corman V M, et al. Detection of 2019 novel coronavirus (2019-nCoV) by real-time R T-PCR. Euro Surveill 25, (2020).
138. Alexandersen S, Chamings A, Bhatta T R. SARS-CoV-2 genomic and subgenomic RNAs in diagnostic samples are not an indicator of active replication. Nat Commun 11, 6059 (2020).
139. Elong Ngono A, et al. Mapping and Role of the CD8(+) T Cell Response During Primary Zika Virus Infection in Mice. Cell Host Microbe 21, 35-46 (2017).

What is claimed:

1. A composition comprising:
a protein or peptide, or variant, homologue, derivative or subsequence thereof, that comprises, consists or consists essentially of a Coronavirus T cell epitope or a nucleic acid molecule encoding the protein or peptide, or variant, homologue, derivative or subsequence thereof;
wherein the composition elicits, stimulates, induces, promotes, increases or enhances a T cell response against two or more different species of Coronavirus.

2. The composition of claim 1, wherein the composition elicits, stimulates, induces, promotes, increases or enhances an antibody response against two or more different species of Coronavirus and a T cell response against two or more different species of Coronavirus.

3. The composition of claim 1 or claim 2, wherein the protein or peptide, or variant, homologue, derivative or subsequence thereof, that comprises, consists or consists essentially of a Coronavirus T cell epitope, is a Coronavirus spike, nucleoprotein, membrane, receptor-binding domains (RBD), replicase polyprotein 1ab, protein 3a, envelope small membrane protein, non-structural protein 3b, protein 7a, protein 9b, non-structural protein 6, or non-structural protein 8a protein or peptide.

4. The composition of claim 3, wherein the protein or peptide, or variant, homologue, derivative or subsequence thereof comprises, consists, or consists essentially of one or more of a Coronavirus spike, nucleoprotein, membrane, envelope, ORF1ab, ORF3a, ORF8, or receptor-binding domain (RBD) protein or peptide.

5. The composition of any one of claims 1 to 4, comprising two or more proteins or peptides, or variants, homologues, derivatives or subsequences thereof, from two or more different species of Coronavirus, or nucleic acid molecules encoding the two or more proteins or peptides, or variants, homologues, derivatives or subsequences thereof, from two or more different species of Coronavirus, wherein the two or more proteins or peptides, or variants, homologues, derivatives or subsequences thereof, comprise, consist or consist essentially of a Coronavirus T cell epitope.

6. The composition of any one of claims 1 to 4, comprising two or more proteins or peptides, or variants, homologues, derivatives or subsequences thereof, from the same species of Coronavirus, or nucleic acid molecules encoding the two or more proteins or peptides, or variants, homologues, derivatives or subsequences thereof, from the same species of Coronavirus, wherein the two or more proteins or peptides, or variants, homologues, derivatives or subsequences thereof, comprise, consist or consist essentially of a Coronavirus T cell epitope.

7. The composition of any one of claim 1 to 5, comprising proteins or peptides, or variants, homologues, derivatives or subsequences thereof from two or more coronavirus subspecies, strains, or variants, or nucleic acid molecules encoding the proteins or peptides, or variants, homologues, derivatives or subsequences thereof, from two or more coronavirus subspecies, strains, or variants,

8. The composition of any one of claims 1 to 7, comprising a protein, variant, homologue, derivative or subsequence thereof from SARS-CoV-2 virus or OC43 virus, or nucleic acid molecules encoding the protein, or variant, homologue, derivative or subsequence thereof, from SARS-CoV-2 virus or OC43 virus.

9. The composition of claim 9, wherein the protein, variant, homologue, derivative or subsequence thereof comprises a Coronavirus virus sequence that is at least 75%, or at least 80%, or at least 85%, or at least 90%, or at least 95% identical to any one of E₂₆₋₄₀, M₈₆₋₁₀₀, M₁₅₁₋₁₆₅, M₁₆₅₋₁₇₉, M₁₆₁₋₁₇₅, M₁₆₆₋₁₈₀, M₁₇₆₋₁₉₀, M₉₁₋₁₀₅, M₃₆₋₅₀, M₁₄₆₋₁₆₀, M₁₃₆₋₁₅₀, M₁₉₁₋₂₀₅, M₁₁₆₋₁₃₀, M₆₆₋₈₀, M₇₁₋₈₅, N₁₀₇₋₁₂₁, N₃₀₃₋₃₁₇, N₁₂₉₋₁₄₃, N₃₂₈₋₃₄₂, N₃₈₇₋₄₀₁, N₂₁₁₋₂₂₅, N₂₁₆₋₂₃₀, N₈₁₋₉₅, N₃₄₆₋₃₆₀, N₃₅₁₋₃₆₅, N₂₆₁₋₂₇₅, N₂₂₁₋₂₃₅, N₃₁₇₋₃₃₁, N₁₂₆₋₁₄₀, N₃₂₆₋₃₄₀, N₃₀₁₋₃₁₅, N₈₆₋₁₀₀, N₁₀₃₋₁₁₃, N₁₀₃₋₁₁₄, N₁₀₃₋₁₁₅, N₁₀₄₋₁₁₃, N₁₀₄₋₁₁₄, N₁₀₄₋₁₁₅, ORF1ab₅₂₄₆₋₅₂₆₀, ORF1ab₅₀₄₁₋₅₀₅₅, ORF3a₁₀₆₋₁₂₀, ORF3a₁₁₆₋₁₃₀, ORF8₈₆₋₁₀₀, ORF8₄₁₋₅₅, ORF8₉₆₋₁₁₀, ORF8₇₆₋₉₀, ORF8₃₆₋₅₀, ORF8₁₀₁₋₁₁₅, S₃₁₅₋₃₂₉, S₅₁₂₋₅₂₆, S₅₃₀₋₅₄₄, S₅₃₉₋₅₅₃, S₈₉₅₋₉₀₉, S₉₅₉₋₉₇₃, S₉₉₈₋₁₀₁₂, and S₁₀₄₄₋₁₀₅₈ from SARS-CoV-2, or as set forth in any one of SEQ ID NOS: 1 to 111, or as set forth in any one of SEQ ID NOS: 1 to 185, multimers, or combinations thereof.

10. The composition of claim 9, wherein the protein, variant, homologue, derivative or subsequence thereof comprises a SARS-CoV-2 virus sequence selected from any one of E₂₆₋₄₀, M₈₆₋₁₀₀, M₁₅₁₋₁₆₅, M₁₆₅₋₁₇₉, M₁₆₁₋₁₇₅, M₁₆₆₋₁₈₀, M₁₇₆₋₁₉₀, M₉₁₋₁₀₅, M₃₆₋₅₀, M₁₄₆₋₁₆₀, M₁₃₆₋₁₅₀, M₁₉₁₋₂₀₅, M₁₁₆₋₁₃₀, M₆₆₋₈₀, M₇₁₋₈₅, N₁₀₇₋₁₂₁, N₃₀₃₋₃₁₇, N₁₂₉₋₁₄₃, N₃₂₈₋₃₄₂, N₃₈₇₋₄₀₁, N₂₁₁₋₂₂₅, N₂₁₆₋₂₃₀, N₈₁₋₉₅, N₃₄₆₋₃₆₀, N₃₅₁₋₃₆₅, N₂₆₁₋₂₇₅, N₂₂₁₋₂₃₅, N₃₁₇₋₃₃₁, N₁₂₆₋₁₄₀, N₃₂₆₋₃₄₀, N₃₀₁₋₃₁₅, N₈₆₋₁₀₀, N₁₀₃₋₁₁₃, N₁₀₃₋₁₁₄, N₁₀₃₋₁₁₅, N₁₀₄₋₁₁₃, N₁₀₄₋₁₁₄, N₁₀₄₋₁₁₅, ORF1ab₅₂₄₆₋₅₂₆₀, ORF1ab₅₀₄₁₋₅₀₅₅, ORF3a₁₀₆₋₁₂₀, ORF3a₁₁₆₋₁₃₀, ORF8₈₆₋₁₀₀, ORF8₄₁₋₅₅, ORF8₉₆₋₁₁₀, ORF8₇₆₋₉₀, ORF8₃₆₋₅₀, ORF8₁₀₁₋₁₁₅, S₃₁₅₋₃₂₉, S₅₁₂₋₅₂₆, S₅₃₀₋₅₄₄, S₅₃₉₋₅₅₃, S₅₄₄₋₅₅₈, S₈₉₅₋₉₀₉, S₉₅₉₋₉₇₃, S₉₉₈₋₁₀₁₂, and S₁₀₄₄₋₁₀₅₈, or as set forth in any one of SEQ ID NOS: 1 to 111, or as set forth in any one of SEQ ID NOS: 1 to 185, multimers, or combinations thereof.

11. The composition of any one of claims 1 to 10, comprising at least two of the spike, nucleoprotein, membrane, envelope, ORF1ab, ORF3a, ORF8, or receptor-binding domain (RBD) proteins or peptides, or variants, homologues, derivatives or subsequences thereof, from a Coronavirus species, or nucleic acid molecules encoding the premembrane and envelope proteins or peptides, or variants, homologues, derivatives or subsequences thereof, from a Coronavirus species.

12. The composition of any one of claims 1 to 11, comprising at least two of the spike, nucleoprotein, membrane, envelope, ORF1ab, ORF3a, ORF8, or receptor-binding domain (RBD) proteins or peptides, or variants, homologues, derivatives or subsequences thereof, from a coronavirus subspecies, strains, or variants, or nucleic acid molecules encoding at least two of the spike, nucleoprotein, membrane, envelope, ORF1ab, ORF3a, ORF8, or receptor-binding domain (RBD) proteins or peptides, or variants, homologues, derivatives or subsequences thereof, from a coronavirus subspecies, strains, or variant.

13. The composition of claim 12, further comprising at least two of the spike, nucleoprotein, membrane or receptor binding-domain (RBD) proteins or peptides, or a variants, homologues, derivatives or subsequences thereof, from SARS-CoV-2 virus or OC43 virus, or nucleic acid molecules encoding at least two of the spike, nucleoprotein, membrane, envelope, ORF1ab, ORF3a, ORF8, or receptor-binding domain (RBD) proteins or peptides, or variants, homologues, derivatives or subsequences thereof, from SARS-CoV-2 virus or OC43 virus.

14. The composition of any one of claims 1 to 13, comprising a protein, or variant, homologue, derivative or subsequence thereof, that comprises, consists or consists essentially of a consensus or representative amino acid sequence derived from proteins or peptides from two or more different species of Coronavirus or proteins or peptides from two or more different species of Coronavirus, or nucleic acid molecules encoding the consensus or representative amino acid sequence derived from proteins or peptides from two or more different species of Coronavirus or proteins or peptides from two or more different species of Coronavirus.

15. The composition of any one of claims 1 to 14, comprising a protein, or variant, homologue, derivative or subsequence thereof, that comprises, consists or consists essentially of a consensus or representative sequence derived from proteins or peptides from two or more of SARS-CoV-2, MERS-CoV, SARS-CoV, OC43, or another coronavirus subspecies, strains, or variants, or nucleic acid molecules encoding the consensus or representative sequence derived from proteins or peptides from two or more of SARS-CoV-2, MERS-CoV, SARS-CoV, OC43, or another coronavirus subspecies, strains, or variant.

16. The composition of claim 14 or claim 15, comprising a protein, or variant, homologue, derivative or subsequence thereof, that comprises, consists or consists essentially of a consensus or representative sequence derived from the spike, nucleoprotein, membrane, envelope, ORF1ab, ORF3a, ORF8, or receptor-binding domain (RBD) proteins or peptides from two or more different species of Coronavirus, or a nucleic acid molecule encoding the protein, or variant, homologue, derivative or subsequence thereof, that comprises, consists or consists essentially of the consensus or representative sequence derived from the spike, nucleoprotein, membrane, envelope, ORF1ab, ORF3a, ORF8, or receptor-binding domain (RBD) proteins or peptides from two or more different species of Coronavirus.

17. The composition of any one of claims 14 to 16, comprising a protein, or variant, homologue, derivative or subsequence thereof, that comprises, consists or consists essentially of a consensus or representative sequence derived from the spike, nucleoprotein, membrane, envelope, ORF1ab, ORF3a, ORF8, or receptor-binding domain (RBD) proteins or peptides from two or more different subspecies, strains, or variants, of coronaviruses, or a nucleic acid molecule encoding the protein, or variant, homologue, derivative or subsequence thereof, that comprises, consists or consists essentially of the consensus or representative sequence derived from the spike, nucleoprotein, membrane, envelope, ORF1ab, ORF3a, ORF8, or receptor-binding domain (RBD) proteins or peptides from two or more different subspecies, strains, or variants, of coronaviruses.

18. The composition of claim 17, comprising a protein, or variant, homologue, derivative or subsequence thereof, that comprises, consists or consists essentially of a consensus or representative sequence derived from the spike, nucleoprotein, membrane, envelope, ORF1ab, ORF3a, ORF8, or receptor-binding domain (RBD) proteins or peptides from SARS-CoV-2 or OC43 and one or more additional subspecies, strains, or variants, of a coronavirus, or a nucleic acid molecule encoding the protein, or variant, homologue, derivative or subsequence thereof, that comprises, consists or consists essentially of a consensus or representative sequence derived from the spike, nucleoprotein, membrane, envelope, ORF1ab, ORF3a, ORF8, or receptor-binding domain (RBD) proteins or peptides from SARS-CoV-2 or OC43 and one or more additional subspecies, strains, or variants, of a coronavirus.

19. The composition of claim 18, wherein the proteins or peptides from SARS-CoV-2 or OC43 and one or more additional subspecies, strains, or variants, of a coronavirus comprise proteins or peptides from two or more species or strains of SARS-CoV-2 or OC43.

20. The composition of claim 18 or 19, comprising a protein, or variant, homologue, derivative or subsequence thereof, that comprises, consists or consists essentially of a consensus or representative sequence derived from the spike, nucleoprotein, membrane, envelope, ORF1ab, ORF3a, ORF8, or receptor-binding domain (RBD) proteins or peptides from SARS-CoV and/or SARS-CoV-2 and/or OC43, or a nucleic acid molecule encoding the protein, or variant, homologue, derivative or subsequence thereof, that comprises, consists or consists essentially of a consensus or representative sequence derived from the spike, nucleoprotein, membrane, envelope, ORF1ab, ORF3a, ORF8, or receptor-binding domain (RBD) proteins or peptides from SARS-CoV and/or SARS-CoV-2 OC43.

21. The composition of claim 19 or 20, comprising a protein, or variant, homologue, derivative or subsequence thereof, that comprises, consists or consists essentially of a consensus or representative sequence derived from the spike, nucleoprotein, membrane, envelope, ORF1ab, ORF3a, ORF8, or receptor-binding domain (RBD) proteins or peptides from MERS-CoV and/or SARS-CoV-2 and/or OC43, or a nucleic acid molecule encoding the protein, or variant, homologue, derivative or subsequence thereof, that comprises, consists or consists essentially of a consensus or representative sequence derived from the spike, nucleoprotein, membrane, envelope, ORF1ab, ORF3a, ORF8, or receptor-binding domain (RBD) proteins or peptides from MERS-CoV and/or SARS-CoV-2 and/or OC43.

22. The composition of any one of claims 1 to 21, further comprising a CD70 protein or peptide, or variant, homologue, derivative or subsequence thereof, or a nucleic acid molecule encoding a CD70 protein, or variant, homologue, derivative or subsequence thereof.

23. The composition of claim 22, wherein the CD70 protein or peptide is a human CD70 protein or peptide, or variant, homologue, derivative or subsequence thereof, or a nucleic acid molecule encoding a human CD70 protein, or variant, homologue, derivative or subsequence thereof.

24. The composition of any one of claims 1 to 23, further comprising a T cell stimulatory protein or peptide, or variant, homologue, derivative or subsequence thereof, or a nucleic acid molecule encoding a T cell stimulatory protein, or variant, homologue, derivative or subsequence thereof.

25. The composition of claim 24, wherein the T cell stimulatory protein or peptide is a human T cell stimulatory protein or peptide, or variant, homologue, derivative or subsequence thereof, or a nucleic acid molecule encoding a human T cell stimulatory protein, or variant, homologue, derivative or subsequence thereof.

26. The composition of claim 24 or 25, wherein the T cell stimulatory protein comprises OX40L, CD70, 4-1BBL, CD40L, GITRL, ICOS-L/B7RP1, CD80/V71, or CD86/B7-2, or a variant thereof.

27. The composition of claim 24 or 25, wherein the T cell stimulatory protein comprises an agonist of OX40, CD27, 4-1BB, CD40, GITR, ICOS, or CD28.

28. The composition of any one of claims 1 to 27, wherein the Coronavirus is one or more of a species or subspecies of Embecovirus, Sarbecovirus, Merbecovirus, Nobevovirus, Hibecovirus, SARS-CoV, MERS-CoV, or OC43.

29. The composition of any one of claims 1 to 28, wherein the Coronavirus is one or more of SARS-CoV, SARS-CoV-2, MERS-CoV, SL-CoV-WIV1, HK84, HKU5, HCoV-OC43, HCoV-HKU1, HKU9, or OC43.

30. The composition of any one of claims 1 to 29, further comprising an adjuvant.

31. The composition of any one of claims 1 to 30, comprising one or more vectors configured to direct expression of the protein, or variant, homologue, derivative or subsequence thereof, that comprises, consists or consists essentially of a Coronavirus T cell epitope.

32. The composition of claim 21 or 22, comprising a vector configured to direct expression of the CD70 protein.

33. The composition of any one of claims 24-27, comprising a vector configured to direct expression of the T cell stimulatory protein.

34. A method of eliciting, stimulating, inducing, promoting, increasing, or enhancing an immune response against a Coronavirus, the method comprising administering the composition of any one of claims 1 to 33.

35. The method of claim 34, wherein the composition of any one of claims 1 to 33 elicits, stimulates, induces, promotes, increases, or enhances an immune response against two or more different species of Coronavirus.

36. The method of claim 34 or claim 35, wherein the method elicits, stimulates, induces, promotes, increases, or enhances an immune response against two or more different species of Coronavirus.

37. A method of vaccinating against, providing a subject with protection against, or treating a subject for a Coronavirus infection, the method comprising administering the composition of any one of claims 1 to 33.

38. The method of claim 37, wherein the method vaccinates against, provides the subject with protection against or treats a subject for infection with two or more different species of Coronavirus.

39. The method of claim 37 or 38, wherein the method vaccinates against, provides the subject with protection against or treats a subject for infection with two or more different subspecies, strains, or variants, of betacoronavirus.

40. The method of any one of claims 34 to 39, wherein the method prevents, reduces, or inhibits sensitizing the subject to or occurrence in the subject of an antibody dependent enhancement of disease or disease upon a secondary or subsequent Coronavirus infection or following administration of the composition of any one of claims 1 to 33 subsequent to a prior Coronavirus infection in the subject or prior to administration to the subject of a vaccine or an immunomodulatory composition against a Coronavirus.

41. A method of formulating a vaccine or an immunomodulatory composition against a Coronavirus that will not elicit, stimulate, induce, promote, increase, enhance or sensitize a subject to an antibody dependent enhancement of disease or infection, the method comprising formulating the vaccine or the immunomodulatory composition to comprise a composition of any one of claims 1 to 33.

42. A nucleic acid vector that expresses the protein or peptide, or variant, homologue, derivative or subsequence thereof, that comprises, consists or consists essentially of a Coronavirus T cell epitope or a nucleic acid molecule encoding the protein or peptide, or variant, homologue, derivative or subsequence thereof of any one of claims 1 to 33.


SEQUENCE LISTING (194 sequences)

SEQ ID NO: 1 - protein, 9 aa, Severe acute respiratory syndrome-related coronavirus
  REGION 1..9: note = misc_feature - Spike
LPPAYTNSF

SEQ ID NO: 2 - protein, 10 aa, Severe acute respiratory syndrome-related coronavirus
  REGION 1..10: note = misc_feature - Spike
YPDKVFRSSV

SEQ ID NO: 3 - protein, 11 aa, Severe acute respiratory syndrome-related coronavirus
  REGION 1..11: note = misc_feature - Spike
YPDKVFRSSVL

SEQ ID NO: 4 - protein, 10 aa, Severe acute respiratory syndrome-related coronavirus
  REGION 1..10: note = misc_feature - Spike
LPFFSNVTWF

SEQ ID NO: 5 - protein, 10 aa, Severe acute respiratory syndrome-related coronavirus
  REGION 1..10: note = misc_feature - Spike
HTPINLVRDL

SEQ ID NO: 6 - protein, 9 aa, Severe acute respiratory syndrome-related coronavirus
  REGION 1..9: note = misc_feature - Spike
TPINLVRDL

SEQ ID NO: 7 - protein, 8 aa, Severe acute respiratory syndrome-related coronavirus
  REGION 1..8: note = misc_feature - Spike
LPQGFSAL

SEQ ID NO: 8 - protein, 11 aa, Severe acute respiratory syndrome-related coronavirus
  REGION 1..11: note = misc_feature - Spike
LPQGFSALEPL

SEQ ID NO: 9 - protein, 10 aa, Severe acute respiratory syndrome-related coronavirus
  REGION 1..10: note = misc_feature - Spike
FPNITNLCPF

SEQ ID NO: 10 - protein, 9 aa, Severe acute respiratory syndrome-related coronavirus
  REGION 1..9: note = misc_feature - Spike
APGQTGKIA

SEQ ID NO: 11 - protein, 11 aa, Severe acute respiratory syndrome-related coronavirus
  REGION 1..11: note = misc_feature - Spike
KPFERDISTEI

SEQ ID NO: 12 - protein, 10 aa, Severe acute respiratory syndrome-related coronavirus
  REGION 1..10: note = misc_feature - Spike
QPYRVVVLSF

SEQ ID NO: 13 - protein, 8 aa, Severe acute respiratory syndrome-related coronavirus
  REGION 1..8: note = misc_feature - Spike
QPYRVVVL

SEQ ID NO: 14 - protein, 9 aa, Severe acute respiratory syndrome-related coronavirus
  REGION 1..9: note = misc_feature - Spike
GPKKSTNLV

SEQ ID NO: 15 - protein, 10 aa, Severe acute respiratory syndrome-related coronavirus
  REGION 1..10: note = misc_feature - Spike
TPCSFGGVSV

SEQ ID NO: 16 - protein, 10 aa, Severe acute respiratory syndrome-related coronavirus
  REGION 1..10: note = misc_feature - Spike
VPVAIHADQL

SEQ ID NO: 17 - protein, 9 aa, Severe acute respiratory syndrome-related coronavirus
  REGION 1..9: note = misc_feature - Spike
TPTWRVYST

SEQ ID NO: 18 - protein, 11 aa, Severe acute respiratory syndrome-related coronavirus
  REGION 1..11: note = misc_feature - Spike
TNSPRRARSVA

SEQ ID NO: 19 - protein, 10 aa, Severe acute respiratory syndrome-related coronavirus
  REGION 1..10: note = misc_feature - Spike
NSPRRARSVA

SEQ ID NO: 20 - protein, 9 aa, Severe acute respiratory syndrome-related coronavirus
  REGION 1..9: note = misc_feature - Spike
SPRRARSVA

SEQ ID NO: 21 - protein, 10 aa, Severe acute respiratory syndrome-related coronavirus
  REGION 1..10: note = misc_feature - Spike
SPRRARSVAS

SEQ ID NO: 22 - protein, 8 aa, Severe acute respiratory syndrome-related coronavirus
  REGION 1..8: note = misc_feature - Spike
SPRRARSV

SEQ ID NO: 23 - protein, 10 aa, Severe acute respiratory syndrome-related coronavirus
  REGION 1..10: note = misc_feature - Spike
RARSVASQSI

SEQ ID NO: 24 - protein, 9 aa, Severe acute respiratory syndrome-related coronavirus
  REGION 1..9: note = misc_feature - Spike
SIIAYTMSL

SEQ ID NO: 25 - protein, 10 aa, Severe acute respiratory syndrome-related coronavirus
  REGION 1..10: note = misc_feature - Spike
AIPTNFTISV

SEQ ID NO: 26 - protein, 9 aa, Severe acute respiratory syndrome-related coronavirus
  REGION 1..9: note = misc_feature - Spike
IPTNFTISV

SEQ ID NO: 27 - protein, 10 aa, Severe acute respiratory syndrome-related coronavirus
  REGION 1..10: note = misc_feature - Spike
LPVSMTKTSV

SEQ ID NO: 28 - protein, 11 aa, Severe acute respiratory syndrome-related coronavirus
  REGION 1..11: note = misc_feature - Spike
KPSKRSFIEDL

SEQ ID NO: 29 - protein, 8 aa, Severe acute respiratory syndrome-related coronavirus
  REGION 1..8: note = misc_feature - Spike
KPSKRSFI

SEQ ID NO: 30 - protein, 9 aa, Severe acute respiratory syndrome-related coronavirus
  REGION 1..9: note = misc_feature - Spike
MIAQYTSAL

SEQ ID NO: 31 - protein, 9 aa, Severe acute respiratory syndrome-related coronavirus
  REGION 1..9: note = misc_feature - Spike
RAAEIRASA

SEQ ID NO: 32 - protein, 10 aa, Severe acute respiratory syndrome-related coronavirus
  REGION 1..10: note = misc_feature - Spike
FPQSAPHGVV

SEQ ID NO: 33 - protein, 11 aa, Severe acute respiratory syndrome-related coronavirus
  REGION 1..11: note = misc_feature - Spike
FPQSAPHGVVF

SEQ ID NO: 34 - protein, 9 aa, Severe acute respiratory syndrome-related coronavirus
  REGION 1..9: note = misc_feature - Spike
FPQSAPHGV

SEQ ID NO: 35 - protein, 8 aa, Severe acute respiratory syndrome-related coronavirus
  REGION 1..8: note = misc_feature - Spike
APHGVVFL

SEQ ID NO: 36 - protein, 9 aa, Severe acute respiratory syndrome-related coronavirus
  REGION 1..9: note = misc_feature - Spike
FPREGVFVS

SEQ ID NO: 37 - protein, 8 aa, Severe acute respiratory syndrome-related coronavirus
  REGION 1..8: note = misc_feature - Spike
FPREGVFV

SEQ ID NO: 38 - protein, 10 aa, Severe acute respiratory syndrome-related coronavirus
  REGION 1..10: note = misc_feature - Spike
SEPVLKGVKL

SEQ ID NO: 39 - protein, 10 aa, Severe acute respiratory syndrome-related coronavirus
  REGION 1..10: note = misc_feature - Membrane
WPVTLACFVL

SEQ ID NO: 40 - protein, 9 aa, Severe acute respiratory syndrome-related coronavirus
  REGION 1..9: note = misc_feature - Membrane
RLFARTRSM

SEQ ID NO: 41 - protein, 10 aa, Severe acute respiratory syndrome-related coronavirus
  REGION 1..10: note = misc_feature - Membrane
FARTRSMWSF

SEQ ID NO: 42 - protein, 8 aa, Severe acute respiratory syndrome-related coronavirus
  REGION 1..8: note = misc_feature - Membrane
VPLHGTIL

SEQ ID NO: 43 - protein, 8 aa, Severe acute respiratory syndrome-related coronavirus
  REGION 1..8: note = misc_feature - Membrane
RPLLESEL

SEQ ID NO: 44 - protein, 10 aa, Severe acute respiratory syndrome-related coronavirus
  REGION 1..10: note = misc_feature - Membrane
RPLLESELVI

SEQ ID NO: 45 - protein, 9 aa, Severe acute respiratory syndrome-related coronavirus
  REGION 1..9: note = misc_feature - Membrane
HLRIAGHHL

SEQ ID NO: 46 - protein, 9 aa, Severe acute respiratory syndrome-related coronavirus
  REGION 1..9: note = misc_feature - Membrane
LPKEITVAT

SEQ ID NO: 47 - protein, 10 aa, Severe acute respiratory syndrome-related coronavirus
  REGION 1..10: note = misc_feature - Nucleoprotein
APRITFGGPS

SEQ ID NO: 48 - protein, 10 aa, Severe acute respiratory syndrome-related coronavirus
  REGION 1..10: note = misc_feature - Nucleoprotein
RPQGLPNNTA

SEQ ID NO: 49 - protein, 9 aa, Severe acute respiratory syndrome-related coronavirus
  REGION 1..9: note = misc_feature - Nucleoprotein
LPNNTASWF

SEQ ID NO: 50 - protein, 11 aa, Severe acute respiratory syndrome-related coronavirus
  REGION 1..11: note = misc_feature - Nucleoprotein
LKFPRGQGVPI

SEQ ID NO: 51 - protein, 10 aa, Severe acute respiratory syndrome-related coronavirus
  REGION 1..10: note = misc_feature - Nucleoprotein
KFPRGQGVPI

SEQ ID NO: 52 - protein, 9 aa, Severe acute respiratory syndrome-related coronavirus
  REGION 1..9: note = misc_feature - Nucleoprotein
FPRGQGVPI

SEQ ID NO: 53 - protein, 10 aa, Severe acute respiratory syndrome-related coronavirus
  REGION 1..10: note = misc_feature - Nucleoprotein
FPRGQGVPIN

SEQ ID NO: 54 - protein, 11 aa, Severe acute respiratory syndrome-related coronavirus
  REGION 1..11: note = misc_feature - Nucleoprotein
FPRGQGVPINT

SEQ ID NO: 55 - protein, 8 aa, Severe acute respiratory syndrome-related coronavirus
  REGION 1..8: note = misc_feature - Nucleoprotein
FPRGQGVP

SEQ ID NO: 56 - protein, 8 aa, Severe acute respiratory syndrome-related coronavirus
  REGION 1..8: note = misc_feature - Nucleoprotein
PRGQGVPI

SEQ ID NO: 57 - protein, 9 aa, Severe acute respiratory syndrome-related coronavirus
  REGION 1..9: note = misc_feature - Nucleoprotein
RIRGGDGKM

SEQ ID NO: 58 - protein, 10 aa, Severe acute respiratory syndrome-related coronavirus
  REGION 1..10: note = misc_feature - Nucleoprotein
LSPRWYFYYL

SEQ ID NO: 59 - protein, 9 aa, Severe acute respiratory syndrome-related coronavirus
  REGION 1..9: note = misc_feature - Nucleoprotein
SPRWYFYYL

SEQ ID NO: 60 - protein, 10 aa, Severe acute respiratory syndrome-related coronavirus
  REGION 1..10: note = misc_feature - Nucleoprotein
SPRWYFYYLG

SEQ ID NO: 61 - protein, 10 aa, Severe acute respiratory syndrome-related coronavirus
  REGION 1..10: note = misc_feature - Nucleoprotein
RNPANNAAIV

SEQ ID NO: 62 - protein, 10 aa, Severe acute respiratory syndrome-related coronavirus
  REGION 1..10: note = misc_feature - Nucleoprotein
NPANNAAIVL

SEQ ID NO: 63 - protein, 9 aa, Severe acute respiratory syndrome-related coronavirus
  REGION 1..9: note = misc_feature - Nucleoprotein
NPANNAAIV

SEQ ID NO: 64 - protein, 11 aa, Severe acute respiratory syndrome-related coronavirus
  REGION 1..11: note = misc_feature - Nucleoprotein
LPQGTTLPKGF

SEQ ID NO: 65 - protein, 10 aa, Severe acute respiratory syndrome-related coronavirus
  REGION 1..10: note = misc_feature - Nucleoprotein
KKPRQKRTAT

SEQ ID NO: 66 - protein, 9 aa, Severe acute respiratory syndrome-related coronavirus
  REGION 1..9: note = misc_feature - Nucleoprotein
KPRQKRTAT

SEQ ID NO: 67 - protein, 11 aa, Severe acute respiratory syndrome-related coronavirus
  REGION 1..11: note = misc_feature - Nucleoprotein
KPRQKRTATKA

SEQ ID NO: 68 - protein, 8 aa, Severe acute respiratory syndrome-related coronavirus
  REGION 1..8: note = misc_feature - Nucleoprotein
KPRQKRTA

SEQ ID NO: 69 - protein, 10 aa, Severe acute respiratory syndrome-related coronavirus
  REGION 1..10: note = misc_feature - Nucleoprotein
APSASAFFGM

SEQ ID NO: 70 - protein, 15 aa, Severe acute respiratory syndrome-related coronavirus
  REGION 1..15: note = misc_feature - Spike
FLVLLPLVSSQCVNL

SEQ ID NO: 71 - protein, 15 aa, Severe acute respiratory syndrome-related coronavirus
  REGION 1..15: note = misc_feature - Spike
LFLPFFSNVTWFHAI

SEQ ID NO: 72 - protein, 15 aa, Severe acute respiratory syndrome-related coronavirus
  REGION 1..15: note = misc_feature - Spike
VTWFHAIHVSGTNGT

SEQ ID NO: 73 - protein, 15 aa, Severe acute respiratory syndrome-related coronavirus
  REGION 1..15: note = misc_feature - Spike
QSLLIVNNATNVVIK

SEQ ID NO: 74 - protein, 15 aa, Severe acute respiratory syndrome-related coronavirus
  REGION 1..15: note = misc_feature - Spike
GYFKIYSKHTPINLV

SEQ ID NO: 75 - protein, 15 aa, Severe acute respiratory syndrome-related coronavirus
  REGION 1..15: note = misc_feature - Spike
IGINITRFQTLLALH

SEQ ID NO: 76 - protein, 15 aa, Severe acute respiratory syndrome-related coronavirus
  REGION 1..15: note = misc_feature - Spike
TRFQTLLALHRSYLT

SEQ ID NO: 77 - protein, 15 aa, Severe acute respiratory syndrome-related coronavirus
  REGION 1..15: note = misc_feature - Spike
AYYVGYLQPRTFLLK

SEQ ID NO: 78 - protein, 15 aa, Severe acute respiratory syndrome-related coronavirus
  REGION 1..15: note = misc_feature - Spike
TSNFRVQPTESIVRF

SEQ ID NO: 79 - protein, 15 aa, Severe acute respiratory syndrome-related coronavirus
  REGION 1..15: note = misc_feature - Spike
ATRFASVYAWNRKRI

SEQ ID NO: 80 - protein, 15 aa, Severe acute respiratory syndrome-related coronavirus
  REGION 1..15: note = misc_feature - Spike
ADYSVLYNSASFSTF

SEQ ID NO: 81 - protein, 15 aa, Severe acute respiratory syndrome-related coronavirus
  REGION 1..15: note = misc_feature - Spike
VLSFELLHAPATVCG

SEQ ID NO: 82 - protein, 15 aa, Severe acute respiratory syndrome-related coronavirus
  REGION 1..15: note = misc_feature - Spike
VNFNFNGLTGTGVLT

SEQ ID NO: 83 - protein, 15 aa, Severe acute respiratory syndrome-related coronavirus
  REGION 1..15: note = misc_feature - Spike
IIAYTMSLGAENSVA

SEQ ID NO: 84 - protein, 15 aa, Severe acute respiratory syndrome-related coronavirus
  REGION 1..15: note = misc_feature - Spike
SFCTQLNRALTGIAV

SEQ ID NO: 85 - protein, 15 aa, Severe acute respiratory syndrome-related coronavirus
  REGION 1..15: note = misc_feature - Spike
QKFNGLTVLPPLLTD

SEQ ID NO: 86 - protein, 15 aa, Severe acute respiratory syndrome-related coronavirus
  REGION 1..15: note = misc_feature - Spike
MIAQYTSALLAGTIT

SEQ ID NO: 87 - protein, 15 aa, Severe acute respiratory syndrome-related coronavirus
  REGION 1..15: note = misc_feature - Spike
GWTFGAGAALQIPFA

SEQ ID NO: 88 - protein, 15 aa, Severe acute respiratory syndrome-related coronavirus
  REGION 1..15: note = misc_feature - Spike
QIPFAMQMAYRFNGI

SEQ ID NO: 89 - protein, 15 aa, Severe acute respiratory syndrome-related coronavirus
  REGION 1..15: note = misc_feature - Spike
MAYRFNGIGVTQNVL

SEQ ID NO: 90 - protein, 15 aa, Severe acute respiratory syndrome-related coronavirus
  REGION 1..15: note = misc_feature - Spike
LNTLVKQLSSNFGAI

SEQ ID NO: 91 - protein, 15 aa, Severe acute respiratory syndrome-related coronavirus
  REGION 1..15: note = misc_feature - Spike
SSNFGAISSVLNDIL

SEQ ID NO: 92 - protein, 15 aa, Severe acute respiratory syndrome-related coronavirus
  REGION 1..15: note = misc_feature - Spike
TGRLQSLQTYVTQQL

SEQ ID NO: 93 - protein, 15 aa, Severe acute respiratory syndrome-related coronavirus
  REGION 1..15: note = misc_feature - Spike
QTYVTQQLIRAAEIR

SEQ ID NO: 94 - protein, 15 aa, Severe acute respiratory syndrome-related coronavirus
  REGION 1..15: note = misc_feature - Spike
QQLIRAAEIRASANL

SEQ ID NO: 95 - protein, 15 aa, Severe acute respiratory syndrome-related coronavirus
  REGION 1..15: note = misc_feature - Spike
AAEIRASANLAATKM

SEQ ID NO: 96 - protein, 15 aa, Severe acute respiratory syndrome-related coronavirus
  REGION 1..15: note = misc_feature - Spike
GKGYHLMSFPQSAPH

SEQ ID NO: 97 - protein, 15 aa, Severe acute respiratory syndrome-related coronavirus
  REGION 1..15: note = misc_feature - Membrane
ICLLQFAYANRNRFL

SEQ ID NO: 98 - protein, 15 aa, Severe acute respiratory syndrome-related coronavirus
  REGION 1..15: note = misc_feature - Membrane
VTLACFVLAAVYRIN

SEQ ID NO: 99 - protein, 15 aa, Severe acute respiratory syndrome-related coronavirus
  REGION 1..15: note = misc_feature - Membrane
YRINWITGGIAIAMA

SEQ ID NO: 100 - protein, 15 aa, Severe acute respiratory syndrome-related coronavirus
  REGION 1..15: note = misc_feature - Membrane
MWLSYFIASFRLFAR

SEQ ID NO: 101 - protein, 15 aa, Severe acute respiratory syndrome-related coronavirus
  REGION 1..15: note = misc_feature - Membrane
ASFRLFARTRSMWSF

SEQ ID NO: 102 - protein, 15 aa, Severe acute respiratory syndrome-related coronavirus
  REGION 1..15: note = misc_feature - Membrane
TNILLNVPLHGTILT

SEQ ID NO: 103 - protein, 15 aa, Severe acute respiratory syndrome-related coronavirus
  REGION 1..15: note = misc_feature - Membrane
ILRGHLRIAGHHLGR

SEQ ID NO: 104 - protein, 15 aa, Severe acute respiratory syndrome-related coronavirus
  REGION 1..15: note = misc_feature - Membrane
PKEITVATSRTLSYY

SEQ ID NO: 105 - protein, 15 aa, Severe acute respiratory syndrome-related coronavirus
  REGION 1..15: note = misc_feature - Membrane
TLSYYKLGASQRVAG

SEQ ID NO: 106 - protein, 15 aa, Severe acute respiratory syndrome-related coronavirus
  REGION 1..15: note = misc_feature - Nucleoprotein
RWYFYYLGTGPEAGL

SEQ ID NO: 107 - protein, 15 aa, Severe acute respiratory syndrome-related coronavirus
  REGION 1..15: note = misc_feature - Nucleoprotein
GIIWVATEGALNTPK

SEQ ID NO: 108 - protein, 15 aa, Severe acute respiratory syndrome-related coronavirus
  REGION 1..15: note = misc_feature - Nucleoprotein
NAAIVLQLPQGTTLP

SEQ ID NO: 109 - protein, 15 aa, Severe acute respiratory syndrome-related coronavirus
  REGION 1..15: note = misc_feature - Nucleoprotein
QIAQFAPSASAFFGM

SEQ ID NO: 110 - protein, 15 aa, Severe acute respiratory syndrome-related coronavirus
  REGION 1..15: note = misc_feature - Nucleoprotein
GTWLTYTGAIKLDDK

SEQ ID NO: 111 - protein, 15 aa, Severe acute respiratory syndrome-related coronavirus
  REGION 1..15: note = misc_feature - Nucleoprotein
KKQQTVTLLPAADLD

SEQ ID NO: 112 - protein, 8 aa, Severe acute respiratory syndrome-related coronavirus
  REGION 1..8: note = misc_feature - Membrane
VPLHGTIL

SEQ ID NO: 113 - protein, 8 aa, Severe acute respiratory syndrome-related coronavirus
  REGION 1..8: note = misc_feature - Membrane
RPLLESEL

SEQ ID NO: 114 - protein, 9 aa, Severe acute respiratory syndrome-related coronavirus
  REGION 1..9: note = misc_feature - Membrane
LPKEITVAT

SEQ ID NO: 115 - protein, 13 aa, Severe acute respiratory syndrome-related coronavirus
  REGION 1..13: note = misc_feature - Nucleocapsid
GPQNQRNAPRITF

SEQ ID NO: 116 - protein, 9 aa, Severe acute respiratory syndrome-related coronavirus
  REGION 1..9: note = misc_feature - Nucleocapsid
LPNNTASWF

SEQ ID NO: 117 - protein, 9 aa, Severe acute respiratory syndrome-related coronavirus
  REGION 1..9: note = misc_feature - Nucleocapsid
FPRGQGVPI

SEQ ID NO: 118 - protein, 18 aa, Severe acute respiratory syndrome-related coronavirus
  REGION 1..18: note = misc_feature - Nucleocapsid
LSPRWYFYYLGTGPEAGL

SEQ ID NO: 119 - protein, 10 aa, Severe acute respiratory syndrome-related coronavirus
  REGION 1..10: note = misc_feature - Nucleocapsid
NPANNAAIVL

SEQ ID NO: 120 - protein, 9 aa, Severe acute respiratory syndrome-related coronavirus
  REGION 1..9: note = misc_feature - Nucleocapsid
KPRQKRTAT

SEQ ID NO: 121 - protein, 10 aa, Severe acute respiratory syndrome-related coronavirus
  REGION 1..10: note = misc_feature - Nucleocapsid
APSASAFFGM

SEQ ID NO: 122 - protein, 10 aa, Severe acute respiratory syndrome-related coronavirus
  REGION 1..10: note = misc_feature - ORF1ab
APHGHVMVEL

SEQ ID NO: 123 - protein, 9 aa, Severe acute respiratory syndrome-related coronavirus
  REGION 1..9: note = misc_feature - ORF1ab
VPHVGEIPV

SEQ ID NO: 124 - protein, 10 aa, Severe acute respiratory syndrome-related coronavirus
  REGION 1..10: note = misc_feature - ORF1ab
IPVAYRKVLL

SEQ ID NO: 125 - protein, 8 aa, Severe acute respiratory syndrome-related coronavirus
  REGION 1..8: note = misc_feature - ORF1ab
APKEIIFL

SEQ ID NO: 126 - protein, 9 aa, Severe acute respiratory syndrome-related coronavirus
  REGION 1..9: note = misc_feature - ORF1ab
TPVVQTIEV

SEQ ID NO: 127 - protein, 11 aa, Severe acute respiratory syndrome-related coronavirus
  REGION 1..11: note = misc_feature - ORF1ab
KPHNSHEGKTF

SEQ ID NO: 128 - protein, 12 aa, Severe acute respiratory syndrome-related coronavirus
  REGION 1..12: note = misc_feature - ORF1ab
KPVETSNSFDVL

SEQ ID NO: 129 - protein, 9 aa, Severe acute respiratory syndrome-related coronavirus
  REGION 1..9: note = misc_feature - ORF1ab
KPNELSRVL

SEQ ID NO: 130 - protein, 9 aa, Severe acute respiratory syndrome-related coronavirus
  REGION 1..9: note = misc_feature - ORF1ab
VAKSHNIAL

SEQ ID NO: 131 - protein, 9 aa, Severe acute respiratory syndrome-related coronavirus
  REGION 1..9: note = misc_feature - ORF1ab
VPGLPGTIL

SEQ ID NO: 132 - protein, 8 aa, Severe acute respiratory syndrome-related coronavirus
  REGION 1..8: note = misc_feature - ORF1ab
RPDTRYVL

SEQ ID NO: 133 - protein, 9 aa, Severe acute respiratory syndrome-related coronavirus
  REGION 1..9: note = misc_feature - ORF1ab
QPGQTFSVL

SEQ ID NO: 134 - protein, 10 aa, Severe acute respiratory syndrome-related coronavirus
  REGION 1..10: note = misc_feature - ORF1ab
LPFAMGIIAM

SEQ ID NO: 135 - protein, 8 aa, Severe acute respiratory syndrome-related coronavirus
  REGION 1..8: note = misc_feature - ORF1ab
VPANSTVL

SEQ ID NO: 136 - protein, 9 aa, Severe acute respiratory syndrome-related coronavirus
  REGION 1..9: note = misc_feature - ORF1ab
KPYIKWDLL

SEQ ID NO: 137 - protein, 9 aa, Severe acute respiratory syndrome-related coronavirus
  REGION 1..9: note = misc_feature - ORF1ab
FPPTSFGPL

SEQ ID NO: 138 - protein, 11 aa, Severe acute respiratory syndrome-related coronavirus
  REGION 1..11: note = misc_feature - ORF1ab
MPNMLRIMASL

SEQ ID NO: 139 - protein, 8 aa, Severe acute respiratory syndrome-related coronavirus
  REGION 1..8: note = misc_feature - ORF1ab
KPPISFPL

SEQ ID NO: 140 - protein, 9 aa, Severe acute respiratory syndrome-related coronavirus
  REGION 1..9: note = misc_feature - ORF1ab
IPARARVEC

SEQ ID NO: 141 - protein, 10 aa, Severe acute respiratory syndrome-related coronavirus
  REGION 1..10: note = misc_feature - ORF1ab
IPARARVECF

SEQ ID NO: 142 - protein, 11 aa, Severe acute respiratory syndrome-related coronavirus
  REGION 1..11: note = misc_feature - ORF1ab
LEIPRRNVATL

SEQ ID NO: 143 - protein, 10 aa, Severe acute respiratory syndrome-related coronavirus
  REGION 1..10: note = misc_feature - ORF1ab
KPRSQMEIDF

SEQ ID NO: 144 - protein, 9 aa, Severe acute respiratory syndrome-related coronavirus
  REGION 1..9: note = misc_feature - ORF1ab
LPKGIMMNV

SEQ ID NO: 145 - protein, 9 aa, Severe acute respiratory syndrome-related coronavirus
  REGION 1..9: note = misc_feature - ORF1ab
FPLKLRGTA

SEQ ID NO: 146 - protein, 9 aa, Severe acute respiratory syndrome-related coronavirus
  REGION 1..9: note = misc_feature - ORF7
RARSVSPKL

SEQ ID NO: 147 - protein, 9 aa, Severe acute respiratory syndrome-related coronavirus
  REGION 1..9: note = misc_feature - ORF7
SPIFLIVAA

SEQ ID NO: 148 - protein, 9 aa, Severe acute respiratory syndrome-related coronavirus
  REGION 1..9: note = misc_feature - ORF8
EPKLGSLVV

SEQ ID NO: 149 - protein, 15 aa, Severe acute respiratory syndrome-related coronavirus
  REGION 1..15: note = misc_feature - Spike
STDLIKNQCVNFNFN

SEQ ID NO: 150 - protein, 15 aa, Severe acute respiratory syndrome-related coronavirus
  REGION 1..15: note = misc_feature - Envelope
FLLVTLAILTALRLC

SEQ ID NO: 151 - protein, 15 aa, Severe acute respiratory syndrome-related coronavirus
  REGION 1..15: note = misc_feature - Membrane
QFAYANRNRFLYIIK

SEQ ID NO: 152 - protein, 15 aa, Severe acute respiratory syndrome-related coronavirus
  REGION 1..15: note = misc_feature - Membrane
VLAAVYRINWITGGI

SEQ ID NO: 153 - protein, 15 aa, Severe acute respiratory syndrome-related coronavirus
  REGION 1..15: note = misc_feature - Membrane
YRINWITGGIAIAMA

SEQ ID NO: 154 - protein, 15 aa, Severe acute respiratory syndrome-related coronavirus
  REGION 1..15: note = misc_feature - Membrane
CLVGLMWLSYFIASF

SEQ ID NO: 155 - protein, 15 aa, Severe acute respiratory syndrome-related coronavirus
  REGION 1..15: note = misc_feature - Membrane
MWLSYFIASFRLFAR

SEQ ID NO: 156 - protein, 15 aa, Severe acute respiratory syndrome-related coronavirus
  REGION 1..15: note = misc_feature - Membrane
TNILLNVPLHGTILT

SEQ ID NO: 157 - protein, 15 aa, Severe acute respiratory syndrome-related coronavirus
  REGION 1..15: note = misc_feature - Membrane
SELVIGAVILRGHLR

SEQ ID NO: 158 - protein, 15 aa, Severe acute respiratory syndrome-related coronavirus
  REGION 1..15: note = misc_feature - Membrane
RGHLRIAGHHLGRCD

SEQ ID NO: 159 - protein, 15 aa, Severe acute respiratory syndrome-related coronavirus
  REGION 1..15: note = misc_feature - Membrane
IAGHHLGRCDIKDLP

SEQ ID NO: 160 - protein, 15 aa, Severe acute respiratory syndrome-related coronavirus
  REGION 1..15: note = misc_feature - Membrane
IKDLPKEITVATSRT

SEQ ID NO: 161 - protein, 15 aa, Severe acute respiratory syndrome-related coronavirus
  REGION 1..15: note = misc_feature - Membrane
KEITVATSRTLSYYK

SEQ ID NO: 162 - protein, 15 aa, Severe acute respiratory syndrome-related coronavirus
  REGION 1..15: note = misc_feature - Membrane
LSYYKLGASQRVAGD

SEQ ID NO: 163 - protein, 15 aa, Severe acute respiratory syndrome-related coronavirus
  REGION 1..15: note = misc_feature - Membrane
SGFAAYSRYRIGNYK

SEQ ID NO: 164 - protein, 15 aa, Severe acute respiratory syndrome-related coronavirus
  REGION 1..15: note = misc_feature - Nucleoprotein
DDQIGYYRRATRRIR

SEQ ID NO: 165 - protein, 15 aa, Severe acute respiratory syndrome-related coronavirus
  REGION 1..15: note = misc_feature - Nucleoprotein
YYRRATRRIRGGDGK

SEQ ID NO: 166 - protein, 15 aa, Severe acute respiratory syndrome-related coronavirus
  REGION 1..15: note = misc_feature - Nucleoprotein
NKDGIIWVATEGALN

SEQ ID NO: 167 - protein, 15 aa, Severe acute respiratory syndrome-related coronavirus
  REGION 1..15: note = misc_feature - Nucleoprotein
AGNGGDAALALLLLD

SEQ ID NO: 168 - protein, 15 aa, Severe acute respiratory syndrome-related coronavirus
  REGION 1..15: note = misc_feature - Nucleoprotein
DAALALLLLDRLNQL

SEQ ID NO: 169 - protein, 15 aa, Severe acute respiratory syndrome-related coronavirus
  REGION 1..15: note = misc_feature - Nucleoprotein
LLLLDRLNQLESKMS

SEQ ID NO: 170 - protein, 15 aa, Severe acute respiratory syndrome-related coronavirus
  REGION 1..15: note = misc_feature - Nucleoprotein
KRTATKAYNVTQAFG

SEQ ID NO: 171 - protein, 15 aa, Severe acute respiratory syndrome-related coronavirus
  REGION 1..15: note = misc_feature - Nucleoprotein
WPQIAQFAPSASAFF

SEQ ID NO: 172 - protein, 15 aa, Severe acute respiratory syndrome-related coronavirus
  REGION 1..15: note = misc_feature - Nucleoprotein
MSRIGMEVTPSGTWL

SEQ ID NO: 173 - protein, 15 aa, Severe acute respiratory syndrome-related coronavirus
  REGION 1..15: note = misc_feature - Nucleoprotein
PSGTWLTYTGAIKLD

SEQ ID NO: 174 - protein, 15 aa, Severe acute respiratory syndrome-related coronavirus
  REGION 1..15: note = misc_feature - Nucleoprotein
FKDQVILLNKHIDAY

SEQ ID NO: 175 - protein, 15 aa, Severe acute respiratory syndrome-related coronavirus
  REGION 1..15: note = misc_feature - Nucleoprotein
ILLNKHIDAYKTFPP

SEQ ID NO: 176 - protein, 15 aa, Severe acute respiratory syndrome-related coronavirus
  REGION 1..15: note = misc_feature - ORF1ab
SHRFYRLANECAQVL

SEQ ID NO: 177 - protein, 15 aa, Severe acute respiratory syndrome-related coronavirus
  REGION 1..15: note = misc_feature - ORF1ab
LMIERFVSLAIDAYP

SEQ ID NO: 178 - protein, 15 aa, Severe acute respiratory syndrome-related coronavirus
  REGION 1..15: note = misc_feature - ORF3a
LYLYALVYFLQSINF

SEQ ID NO: 179 - protein, 15 aa, Severe acute respiratory syndrome-related coronavirus
  REGION 1..15: note = misc_feature - ORF3a
QSINFVRIIMRLWLC

SEQ ID NO: 180 - protein, 15 aa, Severe acute respiratory syndrome-related coronavirus
  REGION 1..15: note = misc_feature - ORF8
PCPIHFYSKWYIRVG

SEQ ID NO: 181 - protein, 15 aa, Severe acute respiratory syndrome-related coronavirus
  REGION 1..15: note = misc_feature - ORF8
FYSKWYIRVGARKSA

SEQ ID NO: 182 - protein, 15 aa, Severe acute respiratory syndrome-related coronavirus
  REGION 1..15: note = misc_feature - ORF8
IGNYTVSCLPFTINC

SEQ ID NO: 183 - protein, 15 aa, Severe acute respiratory syndrome-related coronavirus
  REGION 1..15: note = misc_feature - ORF8
FTINCQEPKLGSLVV

SEQ ID NO: 184 - protein, 15 aa, Severe acute respiratory syndrome-related coronavirus
  REGION 1..15: note = misc_feature - ORF8
GSLVVRCSFYEDFLE

SEQ ID NO: 185 - protein, 15 aa, Severe acute respiratory syndrome-related coronavirus
  REGION 1..15: note = misc_feature - ORF8
RCSFYEDFLEYHDVR

SEQ ID NO: 186 - protein, 10 aa, unidentified
  REGION 1..10: note = OC43 N104-113
LLPRWYFYYL

SEQ ID NO: 187 - DNA, 26 bp, synthetic construct
  misc_feature 1..26: note = Primer
  source 1..26: mol_type = other DNA
acaggtacgttaatagttaatagcgt

SEQ ID NO: 188 - DNA, 22 bp, synthetic construct
  misc_feature 1..22: note = Primer
  source 1..22: mol_type = other DNA
atattgcagcagtacgcacaca

SEQ ID NO: 189 - DNA, 26 bp, synthetic construct
  misc_feature 1..26: note = Primer
  source 1..26: mol_type = other DNA
acactagccatccttactgcgcttcg

SEQ ID NO: 190 - DNA, 24 bp, synthetic construct
  misc_feature 1..24: note = Primer
  source 1..24: mol_type = other DNA
tcccaggtaacaaaccaaccaact

SEQ ID NO: 191 - DNA, 20 bp, synthetic construct
  misc_feature 1..20: note = Primer
  source 1..20: mol_type = other DNA
aaatggtgaattgccctcgt

SEQ ID NO: 192 - DNA, 33 bp, synthetic construct
  misc_feature 1..33: note = Primer
  source 1..33: mol_type = other DNA
cagtacttttaaaagaccttgctcttctggaac

SEQ ID NO: 193 - DNA, 22 bp, synthetic construct
  misc_feature 1..22: note = Primer
  source 1..22: mol_type = other DNA
aatgtaaagatggccgcgtatt

SEQ ID NO: 194 - DNA, 25 bp, synthetic construct
  misc_feature 1..25: note = Primer
  source 1..25: mol_type = other DNA
atgttaacctttaattgaggactat